(19) United States
(12) Patent Application Publication
(10) Pub. No.: US 2009/0149398 A1
(43) Pub. Date: Jun. 11, 2009

(54) 6,11-3c-bicyclic 8a-azalide derivatives

(76) Inventors: Kenneth F. Bartizal, Milford, NJ (US); Milton L. Hammond, Somerville, NJ (US); Dennis M. Schmatz, Cranford, NJ (US); Robert R. Wilkening, Maplewood, NJ (US)

Correspondence Address: MERCK AND CO., INC, P O BOX 2000, RAHWAY, NJ, 07065-0907, US

(21) Appl. No.: 10/586,415

(22) PCT Filed: Jan. 19, 2005
(86) PCT No.: PCT/US2005/001647

§ 371 Date: Jul. 18, 2006

Related U.S. Application Data
(60) Provisional application No. 60/538,822, filed on Jan. 23, 2004.

(51) Int. Cl. A61K 31/7048; C07H 17/08; A61P 3/04; C07H 17/00

(52) U.S. Cl. 514/29; 536/7.1; 514/28

(57) ABSTRACT

Compounds of Formula (I), and pharmaceutically acceptable salts, esters, and prodrugs thereof: (I) are disclosed, wherein A, B, D, L, X, Y, Z and R_{2′}, are described herein. The compounds exhibit antibacterial properties. The compounds of Formula (I) can be employed to treat or prevent bacterial infections as compounds per se or in the form of pharmaceutically acceptable salts, esters, or prodrugs. The compounds and their salts, esters, and prodrugs can also be employed as ingredients in pharmaceutical compositions, optionally in combination with other antibacterial agents, for the treatment of bacterial infections. Processes for making the compounds are also disclosed.

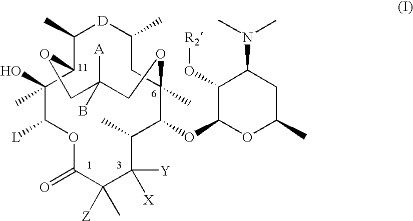

### FIELD OF THE INVENTION

The present invention relates to novel semisynthetic macrolides having antibacterial activity and useful in the treatment and prevention of bacterial infections. More particularly, the invention relates to 6, 11-3C-bridged 8a-azalide derivatives, compositions comprising such compounds, methods for using the same, and processes by which to make such compounds.

### BACKGROUND OF THE INVENTION

Macrolide antibacterial agents are widely used to treat and prevent bacterial infections. However, the discovery of bacterial strains which have resistance or insufficient susceptibility to macrolide antibacterial agents has promoted development of compounds with modified or improved profiles of antibiotic activity. One such class of compounds are azalides, which includes azithromycin, described in U.S. Pat. No. 4,474,768 and U.S. Pat. No. 4,517,359. Azalides are macrolide antibacterial agents with a ring structure similar to the erythronolide A or B, but azalides possess a substituted or unsubstituted nitrogen moiety, such as at the 8a position or at the 9a position as illustrated in the following structures:

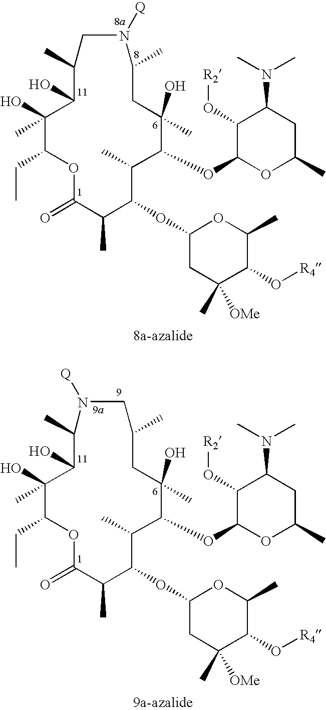

The potential for azalides to display modified or improved profiles for antibiotic activity has spawned extensive research to identify additional azalide derivatives with enhanced clinical properties.

The following references are of interest as background:

US 2004/0157787, published Aug. 12, 2004, US 2004/0053861, published Mar. 19, 2004, and US 2004/0171818 discloses a series of 6-11 bicyclic ketolide derivatives and processes for making the same derivatives;

U.S. Pat. Nos. 6,764,998 and 6,645,941 disclose bridged 9a-azalides that are useful in the treatment and prevention of bacterial;

U.S. Pat. No. 5,866,549 discloses 6-0 substituted ketolides having antibacterial activity;

WO98/56801, published Dec. 17, 1998 discloses a series of 9a-(N-(alkyl))-azalide erythromycin A derivatives and a series of 9a-(N-(alkyl))-azalide 6-O-methylerythromycin A derivatives;

WO98/56802, published Dec. 17, 1998 discloses a series of 9a-N-(H))-azalide erythromycin A derivatives and a series of 9a-(N—(H))-azalide 60-methylerythromycin A derivatives;

WO99/00124, published Jan. 7, 1999 discloses a series of 9a-N-(Rₙ))-azalide 3-thioxoerythromycin A derivatives and a series of 9a-(N—(Rₙ))-azalide 6-O-methyl 3-oxoerythromycin A derivatives, wherein Rₙ is an optionally substituted alkyl or heteroalkyl;

WO99/00125, published Jan. 7, 1999 discloses a series of 9a-N-(Rₙ))-azalide 3-oxoerythromycin A derivatives and a series of 9a-N-(Rₙ))-azalide 6-O-methyl 3-oxoerythromycin A derivatives, wherein Rₙ is an optionally substituted alkyl or heteroalkyl; and

WO 99/19331 discloses 8a-azalides that are potent antibiotics useful for the treatment of gram positive and gram negative organisms.

U.S. Pat. No. 5,686,587 discloses a synthesis of azithromycin comprising introducing a 9a-(N(H))-moiety to erythromycin A by oxime formation, Beckmann rearrangement, reduction, and methylation.

U.S. Pat. No. 5,985,844 discloses homoerythromycin A derivatives modified at the 4″ and 8a positions useful in the therapy of bacterial infections in mammals.

U.S. Pat. No. 6,054,434 discloses 8a-azalides that are useful in the treatment and prevention of bacterial respiratory and enteric infections in livestock animals, particularly in cattle and swine.

U.S. Pat. No. 6,339,063 discloses 9a-azalides that are useful in the treatment and prevention of bacterial respiratory and enteric infections in livestock animals, particularly in cattle and swine.

U.S. Pat. No. 6,645,941 discloses 6, 11-3C-bicyclic 9a-azalide derivatives that have antibacterial activity and are useful in the treatment and prevention of bacterial infections.

### SUMMARY OF THE INVENTION

The present invention provides a novel class of 6,11-3C-bridged 8a-azalide compounds, and pharmaceutically-acceptable salts, esters, and prodrugs thereof, pharmaceutical compositions comprising at least one compound of the present invention, methods for treating or preventing a bacterial infection in a subject by administering said compounds per se or said pharmaceutical compositions, and processes of making the compounds of the present invention.

One embodiment of the present invention includes compounds of Formula I:

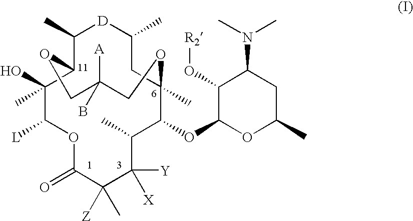

as well as the pharmaceutically acceptable salts, esters and prodrugs thereof, wherein:

### A is

i) —OH;
ii) —ORₚ, where Rₚ is a hydroxy protecting group;
iii) —R₁, where R₁ is aryl, substituted aryl, heteroaryl, or substituted heteroaryl;
iv) —OR₁, where R₁ is as previously defined;
v) —R₂, where R₂ is
   (a) hydrogen;
   (b) halogen;
   (c) —C₁-C₆ alkyl containing 0, 1, 2, or 3 heteroatoms selected from O, S or N, optionally substituted with one or more substituents selected from halogen, aryl, substituted aryl, heteroaryl, or substituted heteroaryl;
   (d) —C₂-C₆ alkenyl containing 0, 1, 2, or 3 heteroatoms selected from O, S, or N, optionally substituted with one or more substituents selected from halogen, aryl, substituted aryl, heteroaryl, or substituted heteroaryl; or
   (e) —C₂-C₆ alkynyl containing 0, 1, 2, or 3 heteroatoms selected from O, S or N, optionally substituted with one or more substituents selected from halogen, aryl, substituted aryl, heteroaryl, or substituted heteroaryl;
vi) —OR₂, where R₂ is previously defined;
vii) —S(O)ₙR₁₁, where n=0, 1 or 2, and R₁ is R₁ or R₂, where R₁ and R₂ are as previously defined;
viii) —NHC(O)R₁₁, where R₁₁ is as previously defined;
ix) —NHC(O)NHR₁₁, where R₁₁ is as previously defined;
x) —NHS(O)₂R₁₁, where R₁₁ is as previously defined;
xi) —NR₁₄R₁₅, where R₁₄ and R₁₅ are each independently R₁₁, where R₁₁ is as previously defined; or
xii) —NHR₃, where R₃ is an amino protecting group;

### B is

i) hydrogen;
ii) deuterium;
iii) halogen;
iv) —OH;
v) —R₁, where R₁ is as previously defined;
vi) —R₂, where R₂ is as previously defined; or
vii) —ORₚ, where Rₚ is as previously defined, provided that when B is halogen, —OH or ORₚ, A is R₁ or R₂, where R₁ and R₂ are previously defined;
   or, alternatively, A and B taken together with the carbon atom to which they are attached are
i) C═O;
ii) C(OR₂)₂, where R₂ is as previously defined;
iii) C(SR₂)₂, where R₂ is as previously defined;
iv) C[—O(CH₂)ₘ]₂, where m=2 or 3;
v) C[—S(CH₂)ₘ]₂, where m is as previously defined;
vi) C═CHR₁₁, where R₁₁ is as previously defined;
vii) C═N—O—R₁₁, where R₁₁ is as previously defined;
viii) C═NNHR₁₁, where R₁₁ is as previously defined;
ix) C═NNHC(O)R₁, where R₁₁ is as previously defined;
x) C═NNHC(O)NHR₁₁, where R₁₁ is as previously defined;
xi) C═NNHS(O)₂R₁₁, where R₁₁ is as previously defined;
xii) C═NNHR₃, where R₃ is as previously defined; or
xiii) C═NR₁₁, where R₁₁ is as previously defined;

### L is

i) —CH₃;
ii) —CH₂CH₃;
iii) —CH(OH)CH₃;
iv) —C₁-C₆ alkyl, optionally substituted with one or more substituents selected from aryl, substituted aryl, heteroaryl, or substituted heteroaryl;
v) —C₂-C₆ alkenyl, optionally substituted with one or more substituents selected from aryl, substituted aryl, heteroaryl, or substituted heteroaryl; or
vi) —C₂-C₆ alkynyl, optionally substituted with one or more substituents selected from aryl, substituted aryl, heteroaryl, or substituted heteroaryl;
   D is —CH₂N(O)—, —C(O)N(R′)—, or —C(OR′)═N—, wherein R′ is R₁₁ as previously defined;

### Q is

i) hydrogen;
   ii) —C₁-C₁₂-alkyl, C₃-C₁₂-alkenyl, or C₃-C₁₂-alkynyl, all optionally substituted with one, two or three substituents independently selected from:
   (a) halogen;
   (b) —OR₆, wherein R₆ is selected from:
      1. hydrogen;
      2. —C₁-C₁₂-alkyl containing 0, 1, 2, or 3 heteroatoms selected from O, S or N, optionally substituted with one, two, or three substituents independently selected from aryl, substituted aryl, heteroaryl, or substituted heteroaryl;
      3. aryl;
      4. substituted aryl;
      5. heteroaryl; and
      6. substituted heteroaryl;
   (c) —NR₄R₅, where R₄ and R₅ are each independently R₆, where R₆ is as previously defined, or in the alternative R₄ and R₅, together with the atom to which they are attached, form a heterocycloalkyl or substituted heterocycloalkyl moiety;
   (d) —N—O—R₆, where R₆ is as previously defined;
   (e) —R₁, where R₁ is as previously defined;
   (f) —C₃-C₈-cycloalkyl;
   (g) substituted —C₃-C₈-cycloalkyl;
   (h) heterocycloalkyl;
   (i) substituted heterocycloalkyl;
   (j) —NHC(O)R₆, where R₆ is as previously defined;
   (k) —NHC(O)OR₇, where R₇ is selected from:
      1. —C₁-C₁₂-alkyl containing 0, 1, 2, or 3 heteroatoms selected from O, S or N, optionally substituted with one, two, or three substituents independently selected from aryl, substituted aryl, heteroaryl, or substituted heteroaryl;
      2. aryl;
      3. substituted aryl;
      4. heteroaryl; or
      5. substituted heteroaryl;
   (l) —NHC(O)NR₄R₅, where R₄ and R₅ are as previously defined;
   (m) —OC(O)NR₄R₅, where R₄ and R₅ are as previously defined;
   (n) —OC(O)R₇, where R₇ is as previously defined;
   (O)—OC(O)OR₇, where R₇ is as previously defined;
   (p) —OC(O)NR₄R₅, where R₄ and R₅ are as previously defined,
   (q) —C(O)R₆, where R₆ is as previously defined,
   (r) —CO₂R₆, where R₆ is as previously defined, or
   (s) —C(O)NR₄R₅, where R₄ and R₅ are as previously defined;
      X is hydrogen;

### Y is

i) hydrogen;
ii) —OH;
iii) —ORₚ, where Rₚ is as previously defined;
iv) —OR₁₁, where R₁₁ is as previously defined;
v) —OC(O)R₁₁, where R₁₁ is as previously defined;
vi) —OC(O)NHR₁₁, where R₁₁ is as previously defined;
vii) —S(O)ₙR₁₁ where n and R₁₁ are as previously defined;
viii)

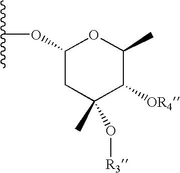

(1) where R₃″ is hydrogen or methyl; R₄″ is hydrogen or Rₚ, where Rₚ is as previously defined;
ix)

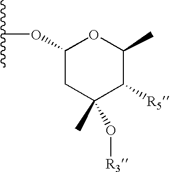

(1) where R₃′ is as previously defined; R₅″ is NH₂ or Ram, where Ram is protected amino;
   or, in the alternative, X and Y are combined together to form oxo;

### Z is

i) hydrogen;
ii) methyl; or
iii) halogen; and
   R₂′ is hydrogen or Rₚ, where Rₚ, is as previously defined.

In another embodiment of the present invention there are disclosed pharmaceutical compositions comprising a therapeutically or prophylactically effective amount of a compound of the invention and a pharmaceutically acceptable carrier or excipient. In yet another embodiment of the invention are methods of treating or preventing antibacterial infections with said pharmaceutical compositions. Suitable carriers and methods of formulation are also disclosed.

In still another embodiment of the present invention there are disclosed pharmaceutical combinations of a first compound of the invention and a second compound of the invention and combinations of a compound of the invention and a known antibacterial agent, wherein in these combinations each active component is employed in an amount that renders the combination effective for treating or preventing a bacterial infection.

In a further aspect of the present invention there are provided processes for the preparation of 6, 11-3C-bridged 8a-azalide derivatives of Formula I.

### DETAILED DESCRIPTION OF THE INVENTION

A first embodiment of the present invention is a compound of Formula I as illustrated above, or a pharmaceutically acceptable salt, ester or prodrug thereof.

Preferred subgenera of compounds of the present invention are:

A compound of Formula II:

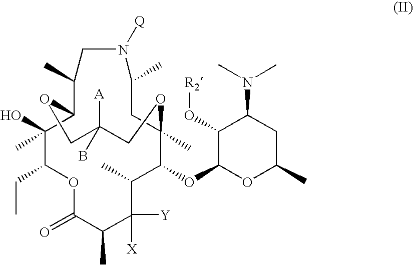

wherein A, B, Q, X, Y, and R₂′ are as previously defined;
A compound of Formula III:

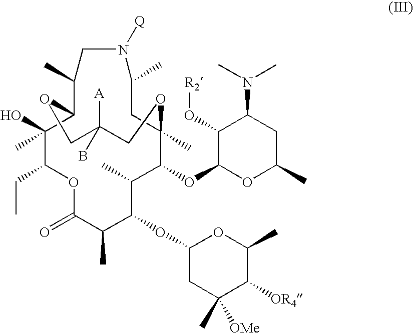

wherein A, B, Q, R₂′, and R₄″ are as previously defined;
A compound of Formula IIIa:

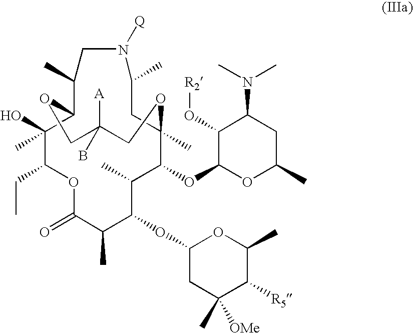

wherein A, B, Q, R₂′, and R₅″ are as previously defined;
A compound of Formula IV:

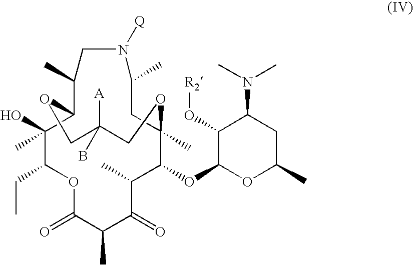

wherein A, B, Q, and R₂′ are as previously defined;
A compound of Formula V:

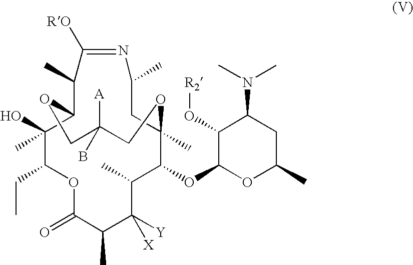

wherein A, B, Q, X, Y, R′, and R₂′ are as previously defined; and
A compound of Formula VI:

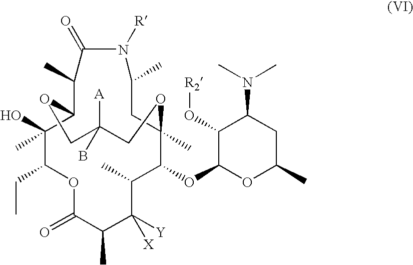

wherein A, B, Q, X, Y, R′, and R₂′ are as previously defined.

Representative compounds according to the invention are those selected from:

A compound of Formula I, wherein A and B taken together with the carbon atom to which they are attached is C═CH₂, D is —CH₂N(Q)-, Q=X=Z=H, Y=OH, L=CH₂CH₃, R₂′=Ac;

A compound of Formula I, wherein A and B taken together with the carbon atom to which they are attached is C═CH₂, D=—CHN(Q)-, Q=Z=H, X and Y taken together are oxo, L=CH₂CH₃, R₂′=H;

A compound of Formula I, wherein A and B taken together with the carbon atom to which they are attached are C═CH₂, D=—CH₂N(Q)-, Q=CH₃, X=Z=H, Y=OH, L=CH₂CH₃, R₂′=H;

A compound of Formula I, wherein A and B taken together with the carbon atom to which they are attached are C═CH₂, D=—CH₂N(Q)-, Q=CH₃, Z=H, X and Y taken together are oxo, L=CH₂CH₃, R₂′=H;

A compound of Formula I, wherein A=H, B=CH₃, D=—CH₂N(Q)-, Q=X=Z=H, Y=OH, L=CH₂CH₃, R₂′=Ac;

A compound of Formula I, wherein A=H, B=CH₃, D=—CH₂N(Q)-, Q=X=Z=H, Y=OH, L=CH₂CH₃, R₂′=H;

A compound of Formula I, wherein A=H, B=CH₃, D=—CHN(Q)-, Q=Z=H, X and Y taken together are oxo, L=CH₂CH₃, R₂′=H;

A compound of Formula I, wherein A=H, B=CH₃, D=—CH₂N(Q)-, Q=CH₃, X=Z=H, Y=OH, L=CH₂CH₃, R₂′=H;

A compound of Formula I, wherein A=H, B=CH₃, D=—CHN(Q)-, Q=CH₃, Z=H, X and Y taken together are oxo, L=CH₂CH₃, R₂′=H;

A compound of Formula I, wherein A=H, B=CH₃, D=—(C═NOH)—, X=Z=H, Y=

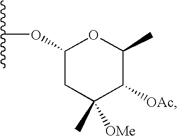

### L=CH₂CH₃, R₂′=Ac;

A compound of Formula I, wherein A=H, B=CH₃, D=—C(═O)NH—, X=Z=H, Y=

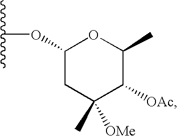

### L=CH₂CH₃, R₂′=Ac;

A compound of Formula I, wherein A=H, B=CH₃, D=C(═O)NH—, X=Z=H, Y=

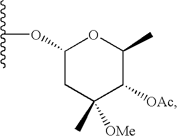

### L=CH₂CH₃, R₂′=H;

A compound of Formula I, wherein A and B taken together with the carbon atom to which they are attached ═C═CH₂, D=—CHN(Q)-, Q=CH₂-Ph, Z=X=H, Y=OH, L=CH₂CH₃, R₂′=H;

A compound of Formula L, wherein A and B taken together with the carbon atom to which they are attached ═C═CH₂, D=—CH₂N(Q)-, Q=CH₂-Ph, Z=H, X and Y are taken together are oxo, L=CH₂CH₃, R₂′=H;

A compound of Formula I, wherein A and B taken together with the carbon atom to which they are attached ═C═CH₂, D=—CH₂N(Q)-, Q=CH₂-(2-pyridyl), Z=X═H, Y=OH, L=CH₂CH₃, R₂′=H;

A compound of Formula I, wherein A and B taken together with the carbon atom to which they are attached ═C═CH₂, D=—CH₂N(Q)-, Q=CH₂-(2-pyridyl), Z=H, X and Y taken together are oxo, L=CH₂CH₃, R₂′=H;

A compound of Formula I, wherein A and B taken together with the carbon atom to which they are attached ═C═C₂, D=—CH₂N(Q)-, Q=CH₂-(3-quinolyl), Z-H, X and Y taken together are oxo, L=CH₂CH₃, R₂′=H;

A compound of Formula I, wherein A and B taken together with the carbon atom to which they are attached ═C═CH₂, D=—CH₂N(Q)-, Q=CH₂-(3-quinolyl), Z=H, X and Y taken together are oxo, L=CH₂CH₃, R₂′=H;

A compound of Formula I, wherein A and B taken together with the carbon atom to which they are attached ═C═CH₂, D=—CH₂N(Q)-, Q=CH₂(CH═CH)-Ph, Z=X═H, Y=OH, L=CH₂CH₃, R₂′=H;

A compound of Formula I, wherein A and B taken together with the carbon atom to which they are attached ═C═CH₂, D=—CHN(Q)-, Q=CH₂(CH═CH)-Ph, Z=H, X and Y taken together are oxo, L=CH₂CH₃, R₂′=H;

A compound of Formula I, wherein A and B taken together with the carbon atom to which they are attached ═C═CH₂, D=—CH₂N(Q)-, Q=CH₂CH═CH-(2-pyridyl), Z=X═H, Y=OH, L=CH₂CH₃, R₂′=H;

A compound of Formula I, wherein A and B taken together with the carbon atom to which they are attached ═C═CH₂, D=—CHN(Q)-, Q=CH₂CH═CH-(2-pyridyl), Z=H, X and Y taken together are oxo, L=CH₂CH₃, R₂′=H;

A compound of Formula I, wherein A and B taken together with the carbon atom to which they are attached ═C═CH₂, D=—CH₂N(Q)-, Q=CH₂C≡C-(3-quinolyl), Z=H, X and Y taken together are oxo, L=CH₂CH₃, R₂′=H;

A compound of Formula I, wherein A and B taken together with the carbon atom to which they are attached ═C═CH₂, D=—CH₂N(Q)-, Q=CH₂C≡C-(3-quinolyl), Z=H, X and Y taken together are oxo, L=CH₂CH₃, R₂′=H;

A compound of Formula I, wherein A and B taken together with the carbon atom to which they are attached ═C═CH—CH═CH-Ph, D=—CH₂N(Q)-, Q=CH₃, Z=H, X and Y taken together are oxo, L=CH₂CH₃, R₂′=H;

A compound of Formula I, wherein A and B taken together with the carbon atom to which they are attached ═C═CH—CH═CH-(3-pyridyl), D=—CH₂N(Q)-, Q=CH₃, Z=H, X and Y taken together are oxo, L=CH₂CH₃, R₂′=H;

A compound of Formula I, wherein A and B taken together with the carbon atom to which they are attached ═C═CH—CH═CH-(3-quinolyl), D=—CH₂N(Q)-, Q=CH₃, Z=H, X and Y taken together are oxo, L=CH₂CH₃, R₂′=H;

A compound of Formula I, wherein A and B taken together with the carbon atom to which they are attached ═C═CH-(3-quinolyl), D=—CH₂N(Q)-, Q=CH₃, Z=H, X and Y taken together are oxo, L=CH₂CH₃, R₂′=H; and

A compound of Formula I, wherein A and B taken together with the carbon atom to which they are attached ═C═CH-Ph, D=—CHN(Q)-, Q=CH₃, Z=H, X and Y taken together are oxo, L=CH₂CH₃, R₂′=H.

Additional representative compounds according to the invention are those selected from:

A compound of Formula I, wherein A and B taken together with the carbon atom to which they are attached is C═CH₂, D is —CH₂N(Q)-, Q=X=Z=H, Y=OH, L=CH₂CH₂CH₃, R₂′=H;

A compound of Formula I, wherein A and B taken together with the carbon atom to which they are attached is C═CH₂, D is —CH₂N(Q)-, Q=CH₂CH₂CH₃, X=Z=H, Y=OH, L=CH₂CH₃, R₂′=H;

A compound of Formula I, wherein A and B taken together with the carbon atom to which they are attached is C═CH₂, D is —CH₂N(Q)-, Q=CH₂CH₂CH₃, Z=H, X and Y taken together are oxo, L=CH₂CH₃, R₂′=H;

A compound of Formula I, wherein A=H, B=CH₃, D=—CH₂N(Q)-, Q CH₂CH₂CH₃, Z=H, X and Y taken together are oxo, L=CH₂CH₂CH₃, R₂′=H;

A compound of Formula I, wherein A and B taken together with the carbon atom to which they are attached is C═O, D is —CH₂N(Q)-, Q=Z=H, X and Y taken together are oxo, L=CH₂CH₃, R₂′=H;

A compound of Formula I, wherein A and B taken together with the carbon atom to which they are attached is C═O, D is —CH₂N(Q)-, Q=CH₃, Z=H, X and Y taken together are oxo, L=CH₂CH₃, R₂′=H;

A compound of Formula I, wherein A and B taken together with the carbon atom to which they are attached is C═O, D is —CH₂N(Q)-, Q=CH₂CH₂CH₃, Z=H, X and Y taken together are oxo, L=CH₂CH₃, R₂′=H;

A compound of Formula I, wherein A and B taken together with the carbon atom to which they are attached is C═N—OH, D is —CH₂N(Q)-, Q=Z=H, X and Y taken together are oxo, L=CH₂CH₃, R₂′=H;

A compound of Formula I, wherein A and B taken together with the carbon atom to which they are attached is C═N—OH, D is —CH₂N(Q)-, Q=CH₃, Z=H, X and Y taken together are oxo, L=CH₂CH₃, R₂′=H;

A compound of Formula I, wherein A and B taken together with the carbon atom to which they are attached is C═N—OH, D is —CH₂N(Q)-, Q=CH₂CH₂CH₃, Z=H, X and Y taken together are oxo, L=CH₂CH₃, R₂′=H;

A compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=Z=H, X and Y taken together are oxo, L=CH₂CH₃, R₂′=H;

A compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=CH₃, Z=H, X and Y taken together are oxo, L=CH₂CH₃, R₂′=H;

A compound of Formula I, wherein A and B taken together with the carbon atom to which they are attached is C═N—O—R₁₁. R₁₁=[5-(6-aminopyrid-2-yl)thien-2-yl]methyl, D is —CH₂N(Q)-, Q=CH₂CH₂CH₃, Z=H, X and Y taken together are oxo, L=CH₂CH₃, R₂′=H;

A compound of Formula I, wherein A and B taken together with the carbon atom to which they are attached is C═N—O—R₁₁. R₁₁=[2-(pyrazol-1-yl)pyrid-5-yl]methyl, D is —CH₂N(Q)-, Q=Z=H, X and Y taken together are oxo, L=CH₂CH₃, R₂′=H;

A compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=CH₃, Z=H, X and Y taken together are oxo, L=CH₂CH₃, R₂′=H;

A compound of Formula I, wherein A and B taken together with the carbon atom to which they are attached is C═N—O—R₁₁. R₁₁=[2-(pyrazol-1-yl)pyrid-5-yl]methyl, D is —CH₂N(Q)-, Q CH₂CH₂CH₃, Z=H, X and Y taken together are oxo, L=CH₂CH₃, R₂′=H;

A compound of Formula I, wherein A and B taken together with the carbon atom to which they are attached is C═N—O—R₁₁, R₁₁=[2-pyrazol-1-yl)pyrid-5-yl]methyl, D is —C═N(Q)-, Q=—C(O)R₆, Z=H, X and Y taken together are oxo, L=CH₂CH₃, R₂′=H;

A compound of Formula I, wherein A and B taken together with the carbon atom to which they are attached is C═N—O—R₁₁, R₁₁=[2-(pyrazol-1-yl)pyrid-5-yl]methyl, D is C═N(Q)-, Q=-acyl, Z=H, X and Y taken together are oxo, L=CH₂CH₃, R₂′=H;

A compound of Formula I, wherein A and B taken together with the carbon atom to which they are attached is C═CH₂, D is —CH₂N(Q)-, Q=X=Z=H, Y=

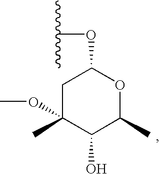

### L=CH₂CH₃, R₂′=H;

A compound of Formula I, wherein A and B taken together with the carbon atom to which they are attached is C═CH₂, D is —CH₂N(Q)-, Q=CH₃, X=Z=H, Y=

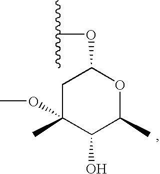

### L=CH₂CH₃, R₂′=H;

A compound of Formula I, wherein A and B taken together with the carbon atom to which they are attached is C═CH₂, D is —CH₂N(Q)-, Q=CH₂CH₂CH₃, X=Z=H, Y=

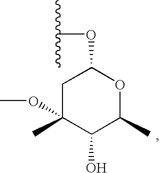

### L=CH₂CH₃, R₂′=H;

A compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=X=Z=H, Y=

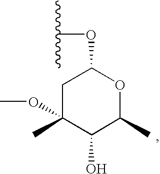

### L=CH₂CH₃; R₂′=H;

A compound of Formula I, wherein A and B taken together with the carbon atom to which they are attached is C═N—R₁₁, R₁₁=[5-(6-aminopyrid-2-yl)thien-2-yl]methyl, D is —CH₂N(Q)-, Q=CH₃, X=Z=H, Y=

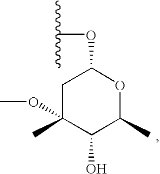

### L=CH₂CH₃, R₂′=H;

A compound of Formula I, wherein A and B taken together with the carbon atom to which they are attached is C═N—O—R₁₁, R₁₁=[5-(6-aminopyrid-2-yl)thien-2-yl]methyl, D is —CH₂N(O)—, Q=CH₂CH₂CH₃, X=Z=H, Y=

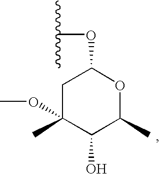

### L=CH₂CH₃, R₂′=H;

A compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q X Z=H, Y=

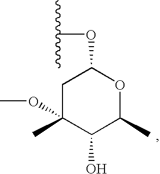

### L=CH₂CH₃, R₂′=H;

A compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=CH₃, X=Z=H, Y=

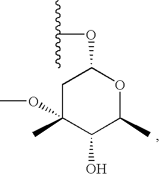

### L=CH₂CH₃, R₂′=H;

A compound of Formula I, wherein A and B taken together with the carbon atom to which they are attached is C═N—O—R₁₁, R₁₁=[2-pyrazol-1-yl)pyrid-5-yl]methyl, D is —CH₂N(Q)-, Q CH₂CH₂CH₃, X=Z=H, Y=

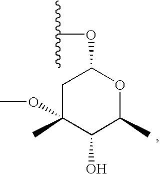

### L=CH₂CH₃, R₂′=H;

A compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=X=Z=H, Y=

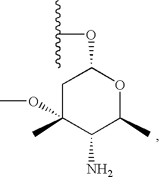

### L=CH₂CH₃, R₂′=H;

A compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=CH₃, X=Z=H, Y=

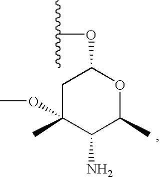

### L=CH₂CH₃, R₂′=H;

A compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=CH₂CH₂CH₃, X=Z=H, Y=

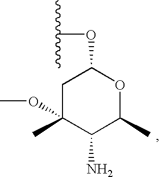

### L=CH₂CH₃, R₂′=H;

A compound of Formula I, wherein A and B taken together with the carbon atom to which they are attached is C═N—O—R₁₁. R₁₁=[2-pyrazol-1-yl)pyrid-5-yl]methyl, D is —CH₂N(Q)-, Q=X=Z=H, Y=

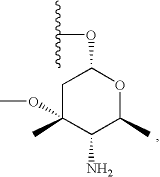

### L=CH₂CH₃, R₂′=H;

A compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=CH₃, X=Z=H, Y=

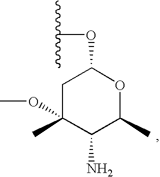

### L=CH₂CH₃, R₂′=H; and

A compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=CH₂CH₂CH₃, X=Z=H, Y=

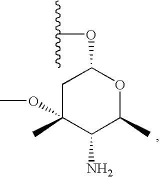

### L=CH₂CH₃, R₂′=H.

Another embodiment of the present invention is a process by which to make compounds of Formula I as previously described.

A further embodiment of the present invention includes pharmaceutical compositions comprising any single compound delineated herein, or a pharmaceutically acceptable salt, ester, or prodrug thereof, and a pharmaceutically acceptable carrier or excipient.

Yet another embodiment of the present invention is a pharmaceutical composition comprising two or more compounds delineated herein, or a pharmaceutically acceptable salt, ester, or prodrug thereof, and a pharmaceutically acceptable carrier or excipient.

Yet a further embodiment of the present invention is a pharmaceutical composition comprising any single compound delineated herein, one or more antibiotics known in the art, or a pharmaceutically acceptable salt, ester, or prodrug thereof, and a pharmaceutically acceptable carrier or excipient.

Still another embodiment of the present invention is a combination of any single compound delineated herein and one or more antibacterial agents known in the art, or a pharmaceutically acceptable salt, ester, or prodrug thereof.

Still another embodiment of the present invention is a combination of two or more of the compounds of the present invention as delineated herein, or a pharmaceutically acceptable salt, ester, or prodrug thereof.

It is understood that a “combination” of a compound of the present invention and one or more other known antibacterial agents or a “combination” of two or more compounds of the present invention means that each of these components can be formulated and/or packaged separately or together and, when formulated separately, that each can be administered concurrently or at different times (e.g., alternately).

Antibiotic agents suitable for use in combination with compounds of the invention include, but are not limited to, carbapenems, penicillins, cephalosporins and other β-lactam antibiotics. When the compounds of Formula I are combined with a carbapenem antibiotic, a dehydropeptidase (DHP) inhibitor may also be combined. Many carbapenems are susceptible to attack by a renal enzyme known as DHP. This attack or degradation may reduce the efficacy of the carbapenem antibacterial agent. Inhibitors of DHP and their use with carbapenems are disclosed in for example EP 0007614, filed Jul. 24, 1979. An exemplary DHP inhibitor is 7-(L-2-amino-2-carboxyethylthio)-2-(2,2-dimethylcyclopropanecarboxamide)-2-heptenoic acid or a useful salt thereof.

A serine β-lactamase inhibitor such as clavulanic acid, sulbactam or tazobactam may also be co-adnistered with the compound of the invention and β-lactam antibiotics, either by separate administration, or co-formulation with one, other or both of the compounds of the invention and the β-lactam antibiotic.

Examples of carbapenems that may be co-administered with the compounds of the invention include, but are not limited to, imipenem, meropenem, biapenem, (4R,5S,6S)-3-[3S,5S)-5-(3-carboxyphenyl-carbamoyl)pyrrolidin-3-ylthio]-6-(1R)-1-hydroxyethyl]-4-methyl-7-oxo-1-azabicyclo[3.2.0]hept-2-ene-2-carboxylic acid, (1S,5R,6S)-2-(4-(2-(((carbamoylmethyl)-1,4-diazoniabicyclo[2.2.2]oct-1-yl)-ethyl(1,8-naphthosultam)methyl)-6-[1(R)hydroxyethyl]-1-methylcarbapen-2-em-3-carboxylate chloride, BMS181139 ([4R-[4alpha,5beta,6beta(R*)]]-4-[2-[(aminoiminomethyl)amino]ethyl]-3-[(2-cyanoethyl)thio]-6-(1-hydroxyethyl)-7-oxo-1-azabicyclo[3.2.0]hept-2-ene-2-carboxylic acid), B02727 ([4R-3[3S*,5S*(R*)],4alpha,5beta,6beta(R*)]]-6-(1-hydroxyethyl)-3-[[5-[1-hydroxy-3-(methylamino)propyl]-3-pyrrolidinyl]thio]-4-methyl-7-oxo-1-azabicyclo[3.2.0]hept-2-ene-2-carboxylic acid monohydrochloride), E1010 ((1R,5S,6S)[1(R)-hydroxymethyl]-2-[2(S)[1(R)-hydroxy-1-[pyrrolidin-3(R)-yl]methyl]pyrrolidin-4(S)-ylsulfanyl]-1-methyl-1-carba-2-penem-3-carboxylic acid hydrochloride) and S4661 ((1R,5S,6S)-2-[(3S,5S)-5-(sulfamoylaminomethyl)pyrrolidin-3-yl]thio-6-[(1R)-1-hydroxyethyl]-1-methylcarbapen-2-em-3-carboxylic acid), (1S,5R,6S)-1-methyl-2-{7-[4-(aminocarbonylmethyl)-1,4-diazoniabicyclo(2.2.2)octan-1yl]-methyl-fluoren-9-on-3-yl}-6-(1R-hydroxyethyl)-carbapen-2-em-3 carboxylate chloride.

Examples of penicillins suitable for co-administration with the compounds according to the invention include benzylpenicillin, phenoxymethylpenicillin, carbenicillin, azidocillin, propicillin, ampicillin, amoxycillin, epicillin, ticarcillin, cyclacillin, pirbenicillin, azloccillin, mezlocillin, sulbenicillin, piperacillin, and other known penicillins. The penicillins may be used in the form of pro-drugs thereof; for example as in vivo hydrolysable esters, for example the acetoxymethyl, pivaloyloxymethyl, α-ethoxycarbonyloxy-ethyl and phthalidyl esters of ampicillin, benzylpenicillin and amoxycillin; as aldehyde or ketone adducts of penicillins containing a 6-α-aminoacetamido side chain (for example hetacillin, metampicillin and analogous derivatives of amoxycillin); and as a-esters of carbenicillin and ticarcillin, for example the phenyl and indanyl α-esters.

Examples of cephalosporins that may be co-administered with the compounds according to the invention include, cefatrizine, cephaloridine, cephalothin, cefazolin, cephalexin, cephacetrile, cephapirin, cephamandole nafate, cephradine, 4-hydroxycephalexin, cephaloglycin, cefoperazone, cefsulodin, ceftazidime, cefuroxime, cefmetazole, cefotaxime, ceftriaxone, and other known cephalosporins, all of which may be used in the form of pro-drugs thereof.

Examples of β-lactam antibiotics other than penicillins and cephalosporins that may be co-administered with the compounds according to the invention include aztreonam, latamoxef (MOXALACTAM), and other known β-lactam antibiotics such as carbapenems like imipenem, meropenem or (4R,5S,6S)-3-[(3S,5S)-5-(3-carboxyphenylcarbamoyl)pyrrolidin-3-ylthio]-6-(1R)-1-hydroxyethyl]-4-methyl-7-oxo-1-azabicyclo[3.2.0]hept-2-ene-2-carboxylic acid, all of which may be used in the form of pro-drugs thereof.

Still another embodiment of the present invention is a compound of the invention or a pharmaceutical composition containing a compound of the invention: (i) for use in, (ii) for use as a medicament for, or (iii) for use in the preparation of a medicament for: (a) inhibiting bacterial growth or (b) preventing or treating a bacterial infection. In these uses, the compounds of the present invention can optionally be employed in combination with one or more known antibacterial agents.

### DEFINITIONS

The terms “C₁-C₃ alkyl, “C₁-C₆ alkyl” or “C₁-C₁₂ alkyl,” as used herein, refer to saturated, straight- or branched-chain hydrocarbon radicals containing from one to three, from one to six, or from one to twelve carbon atoms, respectively. Examples of C₁-C₃ alkyl radicals include methyl, ethyl, propyl and isopropyl radicals; examples of C₁-C₆ alkyl radicals include, but are not limited to, methyl, ethyl, propyl, isopropyl, n-butyl, tert-butyl, neopentyl and n-hexyl radicals; and examples of C₁-C₁₂ alkyl radicals include, but are not limited to, ethyl, propyl, isopropyl, n-hexyl, octyl, decyl, dodecyl radicals.

The terms “C₂-C₁₂ alkenyl” or “C₂-C₆ alkenyl,” as used herein, denote a monovalent group derived from a hydrocarbon moiety containing from two to twelve or two to six carbon atoms and having at least one carbon-carbon double bond. Alkenyl groups include, but are not limited to, for example, ethenyl, propenyl, butenyl, 1-methyl-2-buten-1-yl, and the like.

The terms “C₂-C₁₂ alkynyl” or “C2-CO alkynyl,” as used herein, denote a monovalent group derived from a hydrocarbon moiety containing from two to twelve or two to six carbon atoms and having at least one carbon-carbon triple bond. Representative alkynyl groups include, but are not limited to, for example, ethynyl, 1-propynyl, 1-butynyl, and the like.

The terms “halo” and “halogen,” as used herein, refer to an atom selected from fluorine, chlorine, bromine and iodine.

The term “haloalkyl” denotes an alkyl group, as defined above, having one, two or three halogen atoms attached thereto, and is exemplified by such groups as chloromethyl, bromoethyl, trifluoromethyl, and the like.

The term “aryl,” as used herein, refers to a mono- or bicyclic carbocyclic ring system having one or two aromatic rings including, but not limited to, phenyl, naphthyl, tetrahydronaphthyl, indanyl, indenyl and the like. Aryl groups (including bicyclic aryl groups) can be unsubstituted or substituted with one, two or three substituents independently selected from lower alkyl, substituted lower alkyl, haloakyl, alkoxy, thioalkoxy, amino, alkylamino, dialkylamino, acylamino, cyano, hydroxy, halo, mercapto, nitro, carboxaldehyde, carboxy, alkoxycarbonyl and carboxamide. In addition, substituted aryl groups include tetrafluorophenyl and pentafluorophenyl.

The term “substituted aryl,” as used herein, refers to an aryl group, as defined herein, substituted by independent replacement of one, two or three of the hydrogen atoms thereon with F, Cl, Br, I, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂OH, CH₂CH₂OH, CH₂NH₂, CH₂SO₂CH₃, aryl, heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The term “heteroaryl,” as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

The term “substituted heteroaryl,” as used herein, refers to a heteroaryl group as defined herein, substituted by independent replacement of one, two or three of the hydrogen atoms thereon with F, Cl, Br, I, OH, NO₂, CN, C(O)—C₁-C₆-alkyl, C(O)aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂— heteroaryl, NHCONH₂, NHCONH—C₁-C₆-alkyl, NHCONH-aryl, NHCONH— heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆— alkyl, C₃-C₆-cycloalkyl, CF₃, CH₂CF₃, CHCl₂, CH₂OH, CH₂CH₂OH, CH₂NH₂, CH₂SO₂CH₃, aryl, heteroaryl optionally substituted with amino, benzyl, berizyloxy, aryloxy, heteroaryloxy, C₁-C₆-alkoxy, methoxymethoxy, methoxyethoxy, amino, benzylamino, arylamino, heteroarylamino, C₁-C₃-alkyl-amino, thio, aryl-thio, heteroarylthio, benzyl-thio, C₁-C₆-alkylthio, or methylthiomethyl.

The term “C₃-C₁₂-cycloalkyl” denotes a monovalent group derived from a monocyclic or bicyclic saturated carbocyclic ring compound by the removal of a single hydrogen atom. Examples include, but are not limited to, cyclopropyl, cyclobutyl, cyclopentyl, cyclohexyl, bicyclo[2.2.1]heptyl, and bicyclo[2.2.2]octyl.

The term “substituted C₃-C₁₂-cycloalkyl,” as used herein, refers to a C₃-C₁₂-cycloalkyl group as defined herein, substituted by independent replacement of one, two or three of the hydrogen atoms thereon with F, Cl, Br, L OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)C₁-C₆-alkyl, OC(O)-aryl, OC(O)-heteroaryl, OCO₂-alkyl, OCO₂-aryl, OCO₂— heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂OH, CH₂CH₂OH, CH₂NH₂, CH₂SO₂CH₃, aryl, heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkyl-amino, thio, aryl-thio, beteroarylthio, benzyl-thio, C₁-C₆-alkyl-thio, or methylthiomethyl.

The term “heterocycloalkyl,” as used herein, refers to a non-aromatic 5-, 6- or 7-membered ring or a bi- or tricyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may optionally be oxidized, (iii) the nitrogen heteroatom may optionally be-quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “substituted heterocycloalkyl,” as used herein, refers to a heterocycloalkyl group as defined herein, substituted by independent replacement of one, two or three of the hydrogen atoms thereon with F, Cl, Br, I, OH, NO₂, CN, C(O)—C₁-C₆-alkyl, C(O)-aryl, C(O)-heteroaryl, CO₂-alkyl, CO₂-aryl, CO₂-heteroaryl, CONH₂, CONH—C₁-C₆-alkyl, CONH-aryl, CONIH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-eteroaryl, NHCO₂-alkyl, NHCO₂-aryl, NHCO₂— heteroaryl, NHCONH₂, NHCONH—C₁-C₆-alkyl, NHCONH-aryl, NHCONH— heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂OH, CH₂CH₂OH, CH₂NH₂, CH₂SO₂CH₃, aryl, heteroaryl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkyl-amino, thio, aryl-thio, heteroarylthio, benzyl-thio, C₁-C₆-alkyl-thio, or methylthiomethyl.

The term “C₁-C₆ alkoxy,” as used herein, refers to a C₁-C₆ alkyl group, as previously defined, attached to the parent molecular moiety through an oxygen atom. Examples of C₁-C₆-alkoxy include, but are not limited to, methoxy, ethoxy, propoxy, isopropoxy, n-butoxy, tert-butoxy, neopentoxy and n-hexoxy.

The term “C₁-C₃-alkyl-amino,” as used herein, refers to one or two C₁-C₃-alkyl groups, as previously defined, attached to the parent molecular moiety through a nitrogen atom. Examples of C₁-C₃-alkyl-amino include, but are not limited to, methylamino, dimethylamino, ethylamino, diethylamino, and propylamino.

The term “alkylamino” refers to a group having the structure-NH(C₁-C₁₂ alkyl) where C₁-C₁₂ alkyl is as previously defined.

The term “dialkylamino” refers to a group having the structure-N(C₁-C₁₂ alkyl) (C₁-C₁₂ alkyl), where C₁-C₁₂ alkyl is as previously defined. Examples of dialkylamino are, but not limited to, dimethylamino, diethylamino, methylethylamino, piperidino, and the like.

The term “alkoxycarbonyl” represents an ester group, i.e., an alkoxy group, attached to the parent molecular moiety through a carbonyl group such as methoxycarbonyl, ethoxycarbonyl, and the like.

The term “carboxaldehyde,” as used herein, refers to a group of formula —CHO.

The term “carboxy,” as used herein, refers to a group of formula —COOH.

The term “carboxamide,” as used herein, refers to a group of formula —C(O)NH(C₁-C₁₂ alkyl) or —C(O)N(C₁-C₁₂ alkyl)(C₁-C₁₂ alkyl).

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxy protecting groups include, but are not limited to, methylthiomethyl, tert-dimethylsilyl, tert-butyldiphenylsilyl, acyl substituted with an aromatic group and the like. The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defuied above.

The term “aprotic solvent,” as used herein, refers to a solvent that is relatively inert to proton activity, i.e., not acting as a proton-donor. Examples include, but are not limited to, hydrocarbons, such as hexane and toluene, for example; halogenated hydrocarbons, such as, for example, methylene chloride, ethylene chloride, chloroform, and the like; heterocyclic compounds, such as, for example, tetrahydrofuran and N-methylpyrrolidinone; and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N.Y., 1986.

The term “protogenic organic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Punfication, 4th ed., edited by John A. Riddick et al., Vol. II, in the Techniques of Chemistry Series, John Wiley & Sons, NY, 1986.

“An effective amount,” as used herein, refers to an amount of a compound which confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/kg of body weight to about 500 mg/kg of body weight, preferably from about 1 to about 50 mg/kg. Effective doses will also vary depending on route of administration, as well as the possibility of co-usage with other agents.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, fish, birds and the like. The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain two or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appeig herein is selected for convenience only and is not intended to designate a particular configuration unless the text so states; thus a carbon-carbon double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic anunonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention.

When the compositions of this invention comprise a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

As used herein, unless otherwise indicated, the term “bacterial infection(s)” or “protozoa infections”; includes bacterial infections and protozoa infections that occur in mammals, fish and birds as well as disorders related to bacterial infections and protozoa infections that may be treated or prevented by administering antibiotics such as the compounds of the present invention. Such bacterial infections and protozoa infections and disorders related to such infections include the following: pneumonia, otitis media, sinusitus, bronchitis, tonsillitis, and mastoiditis related to infection by Streptococcus pneumoniae, Haemophilus influenzae, Moraxella catarrhalis, Staphylococcus aureus, or Peptostreptococcus spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasina pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidennidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.; urethritis and cervicitis; and sexually transmitted diseases related to infection by Chlamydia trachomatis, Haeinophilus ducreyi, Treponema pallidum, Ureaplasna urealyticum, or Neserria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chliamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp. odontogenic infection related to infection by viridans streptococci; persistent cough related to infection by Bordetella pertussis; gas gangrene related to infectionpby Clostridiunm perfringens or Bacteroides spp.; and atherosclerosis related to infection by Helicobacter pylori or Chlamydia pneumoniae.

Bacterial infections and protozoa infections and disorders related to such infections that may be treated or prevented in animals include the following: bovine respiratory disease related to infection by P. haem, P. multocida, Mycoplasma bovis, or Bordetella spp.; cow enteric disease related to infection by E. coli or protozoa (i.e., coccidia, cryptosporidia, etc.), dairy cow mastitis related to infection by Staph. aureus, Strep. uberis, Strep. agalactiae, Step. dysgalactiae, Klebsiella spp., Corynebacterium, or Enterococcus spp.; swine respiratory disease related to infection by A. pleuro., P. multocida, or Mycoplasma spp.; swine enteric disease related to infection by E. coli, Lawsonia intracellularis, Salmonella, or Serpulina hyodyisinteriae; cow footrot related to infection by Fusobacterium spp.; cow metritis related to infection by E. coli; cow hairy warts related to Infection by Fusobacterium necrophorum or Bacteroides nodosus; cow pink-eye related to infection by Moraxella bovis, cow premature abortion related to infection by protozoa (i.e. neosporium); urinary tract infection in dogs and cats related to infection by E. coli; skin and soft tissue infections in dogs and cats related to infection by Staph. epidermidis, Staph. interrmedius, coagulase neg. Staph. or P. multocida; and dental or mouth infections in dogs and oats related to infection by Alcaligenes spp., Bacteroides spp., Clostridium spp., Enterobacter spp., Eubacterium, Peptostreptococcus, Porphfyromonas, or Prevotella. Other bacterial infections and protozoa infections and disorders related to such infections that may be treated or prevented in accord with the method of the present invention are referred to in J. P. Sanford at al., “The Sanford Guide To Antimicrobial Therapy,” 26th Edition, (Antimicrobial Therapy, Inc., 1996).

### Antibacterial Activity

The ability of a compound of the present invention to inhibit the growth of bacteria representative of clinical pathogens (e.g., Streptococcus pneumoniae, Streptococcus pyogenes, Staphylococcus aureus, Haemophilus influenzae, and the like) is typically determined by measuring a Minimum Inhibitory Concentration (MIC) of the compound, which is the concentration of the compound needed to inhibit growth of an overnight culture of bacterium of approximately 1×10³ to 1×10⁵ cfi/mL in broth incubated at 37° C. or at another temperature optinal for growth of the pathogen of interest. The compound can, for example, be solubilized in an appropriate diluent and diluted by two-fold dilution in a series of sterile broth tubes or microtiter plates to a level that demonstrates no inhibition of growth in media, such that growth or inhibition of growth of the bacteria can be detected visually. The MIC is the minimum concentration of the compound that will inhibit growth of the bacteria, as compared to a non-compound-treated and non-pathogen inoculated culture.

As an example, compounds of the present invention can be tested for in vitro antibacterial activity by the following method: MIC can be determined in 96 well microtiter plates utilizing the appropriate Mueller Hinton Broth medium (CAMHB) for the observed bacterial isolates. Antimicrobial agents can be serially diluted (2-fold) in bMSO to produce a concentration range from about 64 μg/ml to about 0.03 μg/ml. The diluted compounds (2 μl/well) can then be transferred into sterile, uninoculated CAMHB (0.2 mL) by use of a 96 fixed tip-pipetting station. The inoculum for each bacterial strain can be standardized to 5×10⁵ CFU/mL by optical comparison to a 0.5 McFarland turbidity standard. The plates can then be inoculated with 10 μl/well of adjusted bacterial inoculum. The 96 well plates can then be covered and incubated at a suitable temperature (e.g., 35+/−2° C.) for a suitable time (e.g., 24 hours) in an ambient air environment. Following incubation, plate wells can be visually examined by Optical Density measurement for the presence of growth (turbidity), wherein the lowest concentration of an antimicrobial agent at which no visible growth occurs is defined as the MIC.

A biochemical assessment of the ability of the compounds of the present invention to inhibit protein synthesis in bacteria can be determined using a reaction mix that permits measurement of the in vitro transcription/translation of macromolecules, including protein synthesis. The reaction mix typically contains exogenously added circular DNA encoding a luciferase reporter system that is synthesized in vitro. The S30 extract contains all of the components to synthesize luciferase in an in vitro coupled transcription/translation reaction. In a reaction where protein synthesis is inhibited by a compound luciferase production is inhibited. The measurement protocol typically involves aliquots taken from the S30 transcription translation assays and incubated in the presence of luciferin. Luciferin in the presence of luciferase produces relative light units (rlu). Less rlu are produced when there is less luciferase enzyme due to inhibition of protein synthesis. Light production by luciferase is measured in a luminometer. Such reaction mixes are commercially available as kits, such as E. coli S30 Extract System for Circular DNA available from Promega. Following the protocols provided by the commercial Iit vendor(s), the protein synthesis inhibitory nature of the compound can be quantitated to measure a fifty percent inhibition (IC₅₀), benchmarked against a control protein synthesis inhibitor (i.e. chloramphenical or tetracycline).

The inhibition of specific macromolecules (e.g., DNA, RNA, and protein) in bacteria by compounds of the present invention can be determined in situ in whole, growing bacteria by measuring the level of incorporation of radiolabeled precursors of insoluble macromolecules (i.e. DNA, RNA, protein) in bacteria (e.g., staphylococci, pneumococci, or bacillus). The incorporation of radiolabeled precursors into growing bacteria typically follows the exponential growth of bacteria as measured by optical density in vitro. The addition of compound, added to the growing bacterial culture, with the addition of radionuclides of thymidine, uracil, and methionine (for DNA, RNA, and protein synthesis respectively) can be measured by detecting the incorporation of the specific radiolabel into the insoluble material in the bacterium. Quantitation can be accomplished by either measuring an IC₅₀ of growth compared to no-compound controls, or by using dilutions of compound at the MIC, below the MIC, and above the MIC of the bacterium. The rate of incorporation of a specific label, matched to a specific macromolecule, enables measurement of the chronological inhibition of macromolecules related to the mechanism-of-action of the compound. For example, the measurement of inhibitory activity of a presumptive protein synthesis inhibitor, such as a compound of the present invention, can be observed in vitro in a growing culture by measuring the incorporation of radiolabeled macromolecule precursors in order to determine the transient and selective inhibition of protein synthesis (as detected by 3H-methione incorporation), versus the incorporation of labels indicative of RNA or DNA.

### Pharmaceutical Compositions

The pharmaceutical compositions of the present invention comprise a therapeutically or prophylactically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminun hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. ID addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide—polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, bacterial infections are treated or prevented in a patient such as a human or other animals by administering to the patient a therapeutically or prophylactically effective amount of a compound of the invention, in such amounts and for such time as is necessary to achieve the desired result.

By a “therapeutically effective amount” of a compound of the invention is meant a sufficient amount of the compound to treat bacterial infections, at a reasonable benefit/risk ratio applicable to any medical treatment. By a “prophylactically effective amount” of a compound of the invention is meant an amount sufficient to effect prophylaxis of the disease or condition. It will be understood, however, that the total daily usage of the compounds and compositions of the present invention will be decided by the attending physician within the scope of sound medical judgment. The specific therapeutically or prophylactically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or sy mptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The pharmaceutical compositions of this invention can be administered orally to fish by blending said pharmaceutical compositions into fish feed or said pharmaceutical compositions may be dissolved in water in which infected fish are placed, a method conmionly referred to as a medicated bath. The dosage for the treatment of fish differs depending upon the purpose of administration (prevention or cure of disease) and type of administration, size and extent of infection of the fish to be treated. Generally, a dosage of 5-1000 mg, preferably 20-100 mg, per kg of body weight of fish may be administered per day, either at one time or divided into several times. It will be recognized that the above-specified dosage is only a general range which may be reduced or increased depending upon the age, body weight, condition of disease, etc. of the fish.

Unless otherwise defined, all technical and scientific terms used herein are accorded the meaning commonly known to one with ordinary skill in the art. All publications, patents, published patent applications, and other references mentioned herein are hereby incorporated by reference in their entirety.

### Abbreviations

Abbreviations which appear in the following synthetic schemes and examples include the following: Ac for acetyl; AIBN for azobisisobutyronitrile; Boc for tert-butoxycarbonyl; Bu₃SnH for tributyltin hydride; Bz for benzyl; CDI for carbonyldiimidazole; dba for dibenzylidene acetone; DBU for 1,8-diazabicyclo[5.4.0]undec-7-ene; DEAD for diethylazodicarboxylate; Dess-Martin periodinane for 1,1,1-tris(acetyloxy)-1,1-dihydro-1,2-benziodoxol-3-(1H)-one; DMAP for dimethylaminopyridine; DMF for dimethyl formamide; DMSO for dimethyl sulfoxide; DPPA for diphenylphosphoryl azide; dppb for diphenylphosphino butane; EtOAc for ethyl acetate; iPrOH for isopropanol; NaHMDS for sodium bis(trimethylsilyl)amide; NMO for N-methylmorpholine N-oxide; MeOH for methanol; MOM for methoxymethyl; PDC for pyridinium dichromate; Ph for phenyl; POPd for dihydrogen dichlorobis(di-tert-butylphosphinito-κP) palladate(II); pTSA for p-toluenesulfonic anhydride; TBAHS for tetrabutyl ammonium hydrogen sulfate; TBS for tert-butyl dimethylsilyl; TEA for triethylamine; THF for tetrahydrofuran; TMS for trimethyl silyl; TPAP for tetra-n-propyl ammonium perruthenate; TPP for triphenylphosphine; and Tris for Tris(hydroxymethyl)aminomethane.

### Synthetic Methods

The compounds and processes of the present invention will be better understood in connection with the following synthetic schemes that illustrate the methods by which the compounds of the invention can be prepared.

A preferred intermediate for the preparation of compounds represented by Formula I is a compound represented by the Formula Ia:

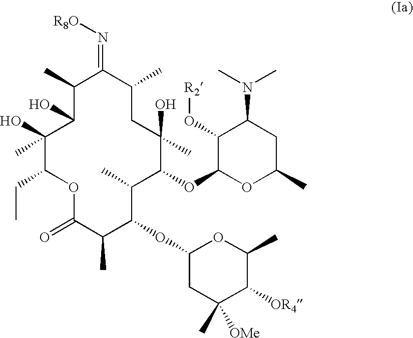

wherein

### 1) R₈ is

a. hydrogen,
b. —CH₂—O—(CH₂)₂OCH₃,
c. —CH₂O(CH₂O)ₙCH₃ where n is as previously defined;
d. —C₁-C₁₂ alkyl, optionally substituted with one or more substituents selected from aryl, substituted aryl, heteroaryl and substituted heteroaryl;
e. —C₃-C₁₂ cycloalkyl;
f. —C(O)—C₁-C₁₂ alkyl;
g. —C(O)—C₃-C₁₂ cycloalkyl;
h. —C(O)—R₁, where R₁ is as previously defined; or
i. —Si(Rₐ)(R_{b})(R_{c}), wherein Rₐ, R_{b} and R_{c} are each independently selected from C₁-C₁₂ alkyl, aryl and substituted aryl; and
   2) R₂′ and R₄″ are as previously defined.

A second preferred intermediate for the preparation of compounds represented by Formula I is a compound represented by the Formula Ib:

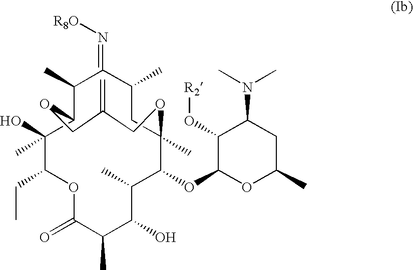

wherein R₂′ is as previously defined.

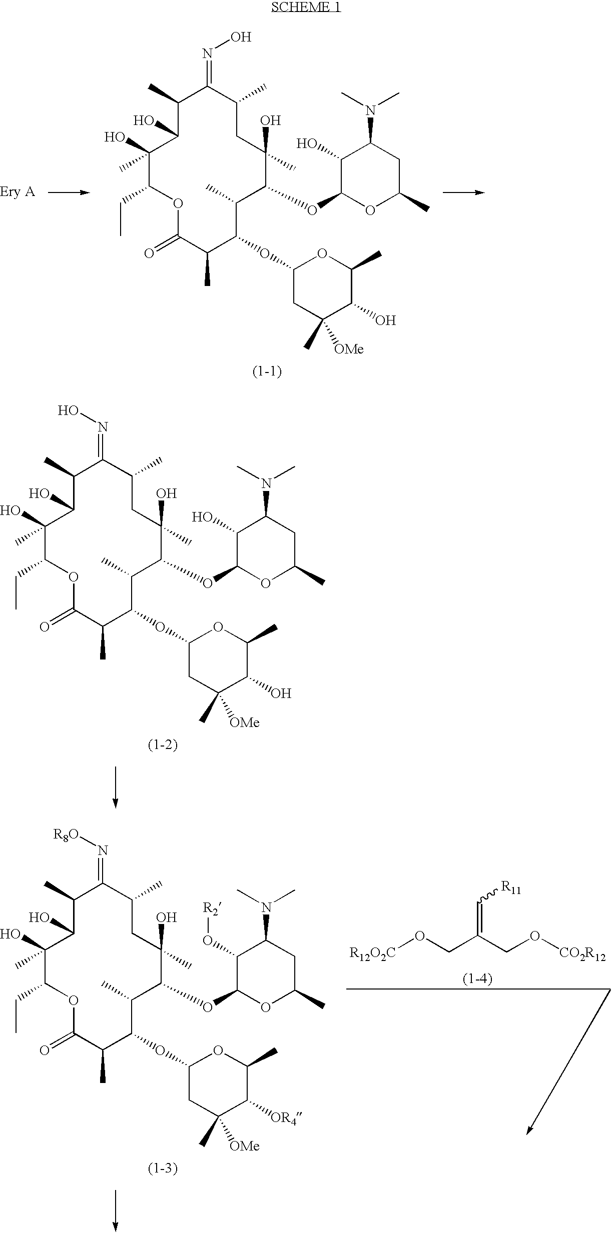

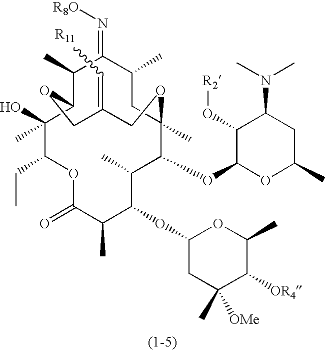

Scheme 1 illustrates a process of the invention, wherein a compound of Formula (I-5) is prepared by reacting a compound of Formula (I-3) with a suitable alkylating agent. In accordance with Scheme 1, the 9-keto group of erythromycins can be converted into an oxime by methods described in U.S. Pat. No. 4,990,602. The E-oxime of erythromycin (I-1) is converted into the Z-oxime of erythromycin (1-2) as described in . . . (need Z-oxime patent reference) This conversion is followed by protection of the 2′- and the 4″-hydroxyl and, if desired, the oxime groups of the erythromycin derivatives to obtain the compounds of Formula (I-3). The preparation of protected erythromycins is also described in U.S. Pat. No. 4,990,602; U.S. Pat. No. 4,331,803; U.S. Pat. No. 4,680,386; U.S. Pat. No. 4,670,549; and EP 260,938.

The 2′- and 4″-hydroxyl are protected by reaction with suitable hydroxyl protecting reagents in an aprotic solvent. Typical hydroxyl protecting reagents include, but are not limited to, acetylating agents, silylating agents, acid anhydrides, and the like. Examples of hydroxyl protecting reagents are, for example, acetyl chloride, acetic anhydride, benzoyl chloride, benzoic anhydride, benzyl-chloroformate, hexamethyldisilazane, and trialkylsilyl chlorides.

Examples of aprotic solvents are dichloromethane, chloroform, tetrahydrofuran, N-methylpyrrolidinone, dimethylsulfoxide, N,N-dimethylformamide, N,N-dimethylacetamide, hexamethylphosphoric triamide, a mixture thereof or a mixture of one of these solvents with ether, tetrahydrofuran, 1,2-dimethoxyethane, 1,2-dichloroethane, acetonitrile, ethyl acetate, acetone, and the like. Aprotic solvents do not adversely affect the reaction. Preferably, the solvent is selected from dichloromethane, chloroform, N,N-dimethylformamide, tetrahydrofuran, N-methylpyrrolidinone, and mixtures thereof. A more thorough discussion of solvents and conditions for protecting the hydroxyl group can be found in T. W. Greene and P. G. M. Wuts, Protective Groups in Organic Synthesis, 3rd ed., John Wiley & Son, Inc, 1999.

Protection of 2′- and 4″-hydroxyl groups may be accomplished sequentially or simultaneously to provide compound (1-3) where R₂′ and/or R₄″ can be, for example, acetyl, benzoyl, trimethylsilyl, and the like. Preferred protecting groups include acetyl, benzoyl, and trimethylsilyl. A particularly preferred group for protecting the hydroxyl and oxime groups is the acetyl protecting group, wherein R₂′=R₄″=R₆=Ac.

Acetylation of the hydroxyl group is typically accomplished by treating the compound (1-2) with an acetylating reagent, for example, acetic anhydride or acetyl chloride.

The erythromycin derivative of Formula (1-3) is then reacted with an alkylating agent of the formula: R₁₂—OC(O)O—CH₂[C═CHR₁₁]CH₂—OC(O)—OR₁₂ (1-4), wherein R₁₂ is C₁-C₁₂ alkyl and R₁₁ is as previously defined.

Most palladium (O) catalysts can be expected to work in this process. Some palladium (II) catalysts, such as palladium (II) acetate, which is converted into a palladium (O) species in-situ by the action of a phosphine, will work as well. See, for example, Beller et al. Angew. Chem. Int. Ed. Engl., 1995, 34 (17), 1848. The palladium catalyst can be selected from, but not limited to, palladium (II) acetate, tetrakis(triphenylphospine)palladium (O), tris(dibenzylideneacetone) dipalladium, tetradibenzylideneacetone)dipalladium and the like. Palladium on carbon and palladium (II) halide catalysts are less preferred than other palladium catalysts for this process. Suitable phosphines include, but are not limited to, triphenylphosphine, bis(diphenylphosphino)methane, bis(diphenylphosphino)ethane, bis(diphenylphosphino)propane, 1,4-bis(diphenylphosphino)butane, bis(diphenylphosphino)pentane, tri-o-tolyl-phosphine, and the like.

The reaction can be carried out in an aprotic solvent, preferably at elevated temperature, preferably at or above about 50° C. Suitable aprotic solvents include, but are not limited to, tetrahydrofuran, N,N-dimethylformamide, dimethyl sulfoxide, N-methyl-2-pyrrolidone, hexamethylphosphoric triamide, 1,2-dimethoxyethane, methyl-tert-butyl ether, heptane, acetonitrile, isopropyl acetate and ethyl acetate. The most preferred solvents are tetrahydrofuran or toluene.

Generally, the alkylating agents have the Formula (1-4) as previously described. The preferred alkylating agents are those wherein R₁₂ is tert-butyl, isopropyl or isobutyl. The alkylating reagents can be prepared by reaction of a diol with a wide variety of compounds for incorporating the dicarbonate moiety. The compounds include, but are not limited to, tert-butyl chloroformate, di-tert-butyl dicarbonate, and 1-(tert-butoxycarbonyl)imidazole, and the reaction can be carried out in the presence of an organic or an inorganic base. The temperature of the reaction can vary from about −30° C. to about 30° C. Preferably the alkylating reagent is di-tert-butyl dicarbonate.

An alternative method of converting the alcohol into the carbonate involves treating the alcohol with phosgene or triphosgene to prepare the chloroformate derivative of the diol. The dichloroformate derivative can then be converted into the dicarbonate by the methods described in Cotarca, L., Delogu, P., Nardelli, A., Sunijic, V, Synthesis, 1996, 553. The reaction can be carried out in a variety of organic solvents such as dichloromethane, toluene, diethyl ether, ethyl acetate and chloroform in the presence of a base. Examples of suitable bases include, but are not limited to, sodium hydroxide, potassium hydroxide, ammonium hydroxide, sodium carbonate, potassium carbonate, ammonium carbonate, DMAP, pyridine, triethylamine and the like. The temperature can vary from about 0° C. to about 60° C. The reaction time can vary over a wide range depending upon the reaction scale, conditions employed and the choice of reagents (e.g., solvent and base), but typically the reaction can be expected to run to completion in from about 3 to about 5 hours.

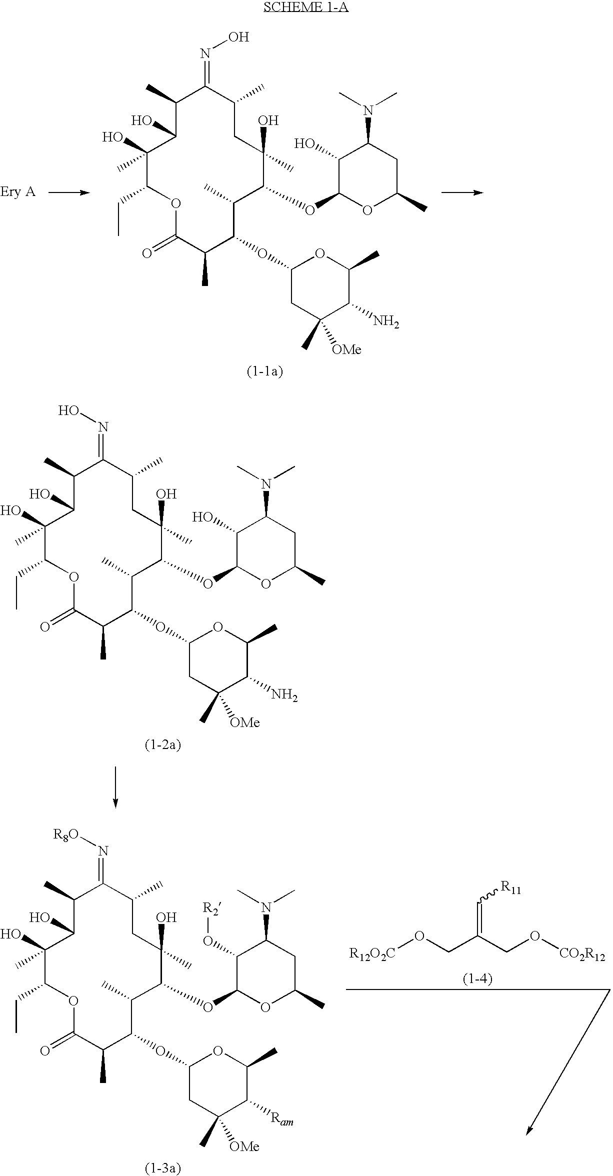

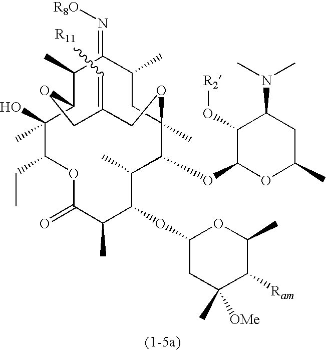

Scheme 1-A is analogous to Scheme 1. It depicts a process for preparing a compound of Formula (1-5a) by reacting a compound of Formula (1-3a) with a suitable alkylating agent. The compounds of Formula (1-1a), (1-2a) and (1-3a) are identical to the compounds of Formula (1-1), (1-2) and (1-3) respectively, except that the 4″-hydroxyl and the 4″-protected hydroxyl in these compounds have been replaced with amino and protected amino and the stereochemistry of the 4″ position has been inverted. The description of the chemistry set forth above for Scheme 1 applies to Scheme 1-Ai except that the preparation of the compound of Formula (1-1a) from Ery A involves different chemistry than that for the preparation of (1-1), and an amino protection step is required in addition to a hydroxyl protection step in order to obtain (1-3a) from (1-2a).

The amino group can be protected by reaction with a suitable amine protecting reagent in a suitable solvent. Typical amine protecting reagents include, but are not limited to, acylating agents, sulfonylating agents, phosphonylating agents, anhydrides, and the like. Treatment with acylating agents results in the formation of carbamate protecting groups, sulfonylating agents result in sulfonamides, phosphonylating agents result in phosphoramidates or phosphinamides, and anhydrides result in carbamates. Suitable amine protecting reagents include, but are not limited to, (C₁₋₆ alkyloxy)carbonyl halides (Boc halides), di-tert-butyl carbonate, di-allyl carbonate, dibenzyl carbonate, benzyloxycarbonyl halides (CBZ halides), allyloxycarbonyl halides (ALLOC halides), diphenylphosphinyl halides, di-(C₁₋₃ alkyl)phosphono halides, diphenylphosphono halides, and dibenzylphosphono halides. Representative examples of amine protecting agents in this class are Ph₂P(═O)Cl, (i-PrO)₂P(═O)Cl, (t-BuO)₂P(═O)Cl, (BnO)₂P(═O)Cl, BOC-Cl, CBZ-Cl, (CBZ)₂O, (ALLOC)₂O, allyl chloroformate, and (BOC)₂O. Particularly suitable amine protecting agents are selected from BOC-halide and (BOC)₂O.

Suitable solvents for the amine protecting step include, but are not limited to, aliphatic and alicyclic hydrocarbons, aromatic hydrocarbons, halogenated aliphatic hydrocarbons, alcohols, esters, ethers, and nitriles. Exemplary solvents include hexane (pure and mixed isomers), cyclohexane, cycloheptane, toluene, single and mixed isomers of xylene, methylene chloride, DCE, chloroform, carbon tetrachloride, methanol, ethanol, isopropanol, n-butanol, t-butanol and iso-butanol, ethyl acetate, isopropyl acetate, isobutyl acetate, n-butyl acetate, THF, diethyl ether, di-n-butyl ether, MTBE, DME, acetonitrile, and propionitrile.

The temperature employed in the amine protection step is suitably in a range of from about −20 to about 60° C., and is typically in a range of from about −20 to about 50° C. (e.g., from about −5 to about 35° C.).

A more thorough discussion of solvents and conditions for protecting the amine group can be found in T. W. Greene and P. G. M. Wuts, Protective Groups in Organic Synthesis, 3rd ed., John Wiley & Son, Inc, 1999.

Protection of 2′-hydroxyl and the 4″-amino can be accomplished sequentially in either order. The erythromycin derivative of Formula (1-3a) can then be reacted with an alkylating agent of the formula: R₁₂—OC(O)O—CH₂[C═CHR₁₁]CH₂—OC(O)—OR₁₂ (1-3), wherein R₁₂ is C₁-C₁₂ alkyl and R₁₁ is as previously defined.

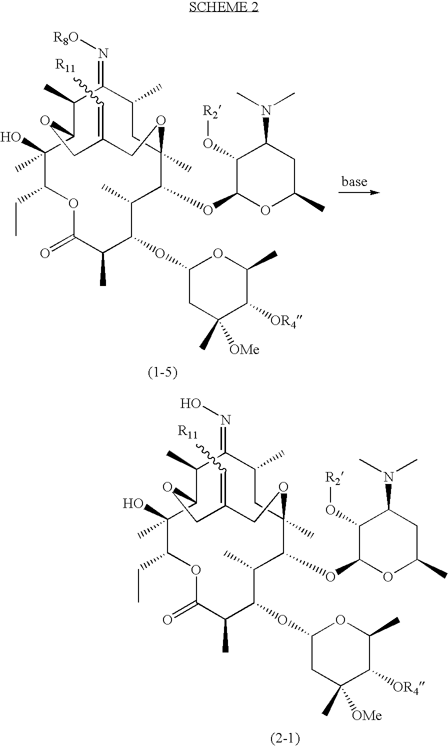

Another process of the invention involves the selective deprotection of the oxime group, wherein the protected Z-oxime of Formula (1-5) is treated with a base to deprotect (i.e., remove protecting group R₈) without isomerization to the E-oxime. Suitable bases include an aqueous solution of lithium hydroxide or sodium hydroxide or the like, preferably in a two-phase system using a base stable solvent such as methylene chloride or the like as the organic phase. The 9Z-oxime of Formula (2-1) can be isolated and purified at this point.

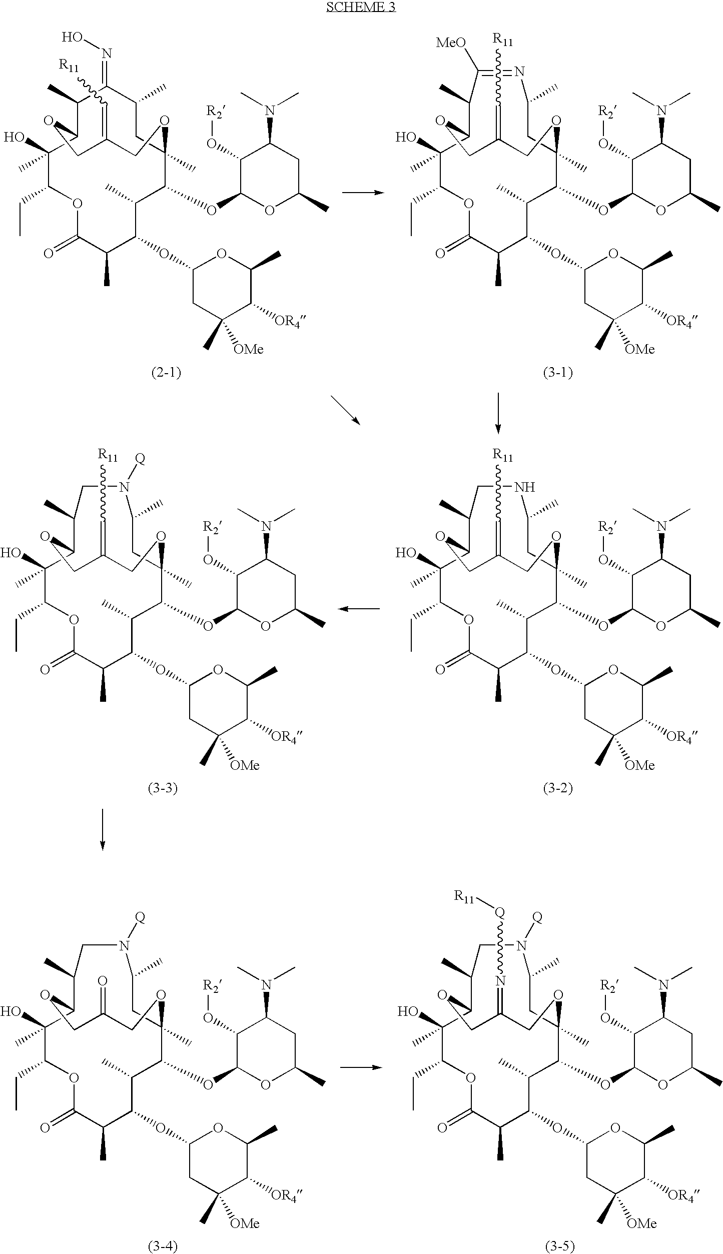

Compound (2-1) where R₂′ is Ac can be converted into the compounds of Formula (3-1) by the Beckmann rearrangement. Thus, the compound of Formula (2-1) can be treated with an oxime activating agent and subsequently quenched by addition of methanol to provide the compounds of Formula (3-1). Representative oxime activating agents include, but are not limited to, sulfonic anhydrides and sulfonyl halides such as p-toluenesulfonic anhydride, methanesulfonic anhydride, p-toluenesulfonyl chloride, methanesulfonyl chloride, p-bromosulfonyl chloride, optionally in the presence of a base such as, but not limited to, pyridine, triethyl amine, diisopropylethyl amine, NaHCO₃, Na₂CO₃, KHCO₃ and K₂CO₃. For further details concerning the Beckmann rearrangement see L. G. Donaruma, W. Z. Heldt, Org. React. 11, 1-156 (1960); R. E. Gawley, ibid. 35, 1420 (1988); C. G. McCarty in The Chemistry of the Carbon-Nitrogen Double Bond, S. Patai, Ed. (Interscience, New York, 1970) pp 408-439; J. R. Hauske, Comp. Org. Syn. 1, 98-100 (1991); K. Maruoka, H. Yamamoto, ibid. 6, 763-775; D. Craig, ibid. 7, 689-702; and U.S. Pat. No. 5,985,844.

Reduction of compounds of Formula (3-1) to compounds of Formula (3-2) can be achieved by treatment of the former with reducing agents including, but not limited to, borane in THF, borane dimethylsulfide, sodium cyanoborohydride, sodium borohydride optionally in the presence of an acid such as TiCl₄, COCl₂, AlCl₃, methanesulfonic acid, or acetic acid. Applicable solvents include, but are not limited to, those selected from tetrahydrofuran, 1,4-dioxane, 1,2-dimethoxyethane, isopropanol, ethanol, butanol acetonitrile, diethyl ether, dichloromethane, water and mixtures thereof. The reaction temperature is from about −78° C. to about 30° C. In a particularly preferred embodiment, compounds of Formula (2-1) can be treated with p-toluenesulfonic anhydride and triethylamine in methylene chloride and subsequently quenched with methanol to provide compounds of Formula (3-1). Compounds of Formula (3-1) can then be treated with NaBH₄ in methanol to provide the compounds of Formula (3-2). This reduction can also be preformed without isolation of the intermediate iminoether (3-1) to provide the azalide (3-2) directly.

Compounds of Formula (3-2) can be converted to compounds of Formula (3-3) by treatment of the former with alkylating agent Q-X₁, wherein X₁ is a halo leaving group, in the presence of base. An alternative means of converting compounds of Formula (3-2) to compounds of Formula (3-3) is treatment of the compounds of Formula (3-2) with an aldehyde Q-CHO in the presence of acetic acid and excess NaCNBH3 to provide compounds of Formula (3-3) where Q is —CH₂R₂, wherein R₂ is as previously defined. Examples of solvents include, but are not limited to, acetonitrile, diethylether, dichloromethane, chloroform, ethyl acetate, THF, dioxane or mixtures thereof. The reaction suitably proceeds at a temperature of from about −20° C. to about 80° C. for about 30 minutes to about 18 hours. In a particularly preferred-embodiment, Q-CHO and formic acid can be reacted with (3-2) in chloroform at about 80° C.

Conversion of alkenes (3-3) into ketones (3-4) can be accomplished by ozonolysis followed by decomposition of the ozonide with the appropriate reducing agents. The reaction can be carried out in an inert solvent such as, but not limited to, methanol, ethanol, ethyl acetate, glacial acetic acid, chloroform, methylene chloride or hexane or mixtures thereof. In a preferred embodiment, the solvent is methanol, and the conversion is conducted at a temperature of from about 78° C. to about −20° C. Representative reducing agents include, for example, those selected from triphenylphosphine, trimethylphosphite, thiourea, and dimethyl sulfide. Triphenylphosphine is a preferred reducing agent. A more thorough discussion of ozonolysis and conditions therefor rnay be found in J. March, Advanced Organic Chemistry, 4th ed., Wiley & Son, Inc, 1992. Alternatively, compounds of Formula (34) can be prepared from compounds of Formula (3-3) dihydroxydation with OsO₄ followed by NaIO₄ cleavage to provide the compounds of Formula of (3-4).

The ketone of Formula (3-4) can be converted into an oxime of Formula (3-5). Oxime formation can be accomplished using the appropriate substituted hydroxylamine under either acidic or basic conditions in a variety of solvents. Representative acids include, but are not limited to, those selected from hydrochloric acid, phosphoric acid, sulfuric acid, p-toluenesulfonic acid, and pyridinium p-toluene sulfonate. Likewise, representative bases include, but are not limited to, those selected from triethylamine, pyridine, diisopropylethyl amine, 2,6-lutidine, and the like. Appropriate solvents include, but are not limited to, methanol, ethanol, water, tetrahydrofuran, 1,2-dimethoxyethane, and ethyl acetate. The reaction can preferably be carried out in ethanol using triethylamine as the base. The reaction temperature is typically about 25° C. and the reaction time is typically from about 1 to about 12 hours.

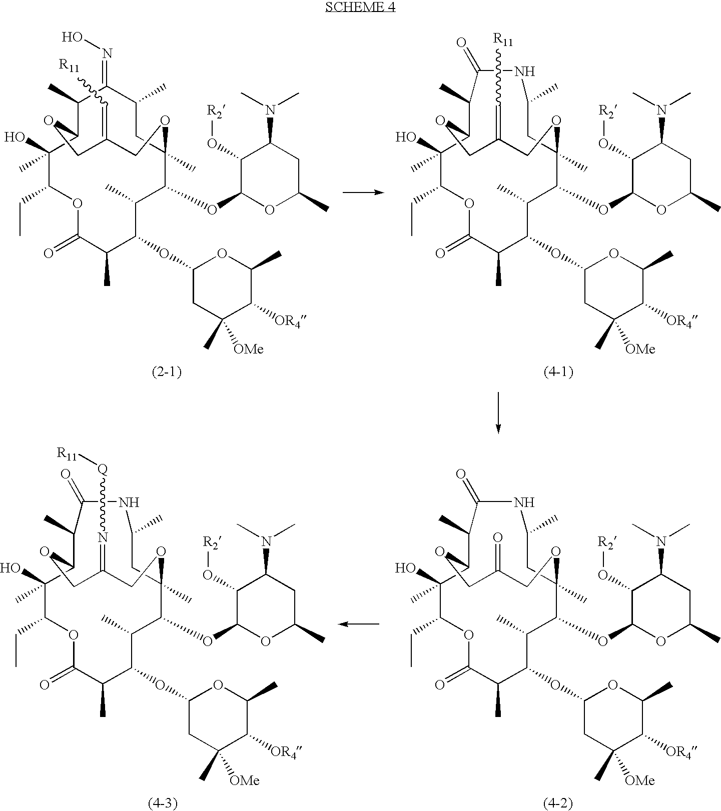

The compounds of Formula (4-1) can be synthesized via treatment of compounds of Formula (2-1) with p-toluenesulfonyl chloride and NaHCO₃ in acetone and water. Conversion of alkenes (4-1) into ketones (4-2) can be accomplished as described previously. The ketone of Formula (4-2) can be converted into an oxime of Formula (4-3) as described previously.

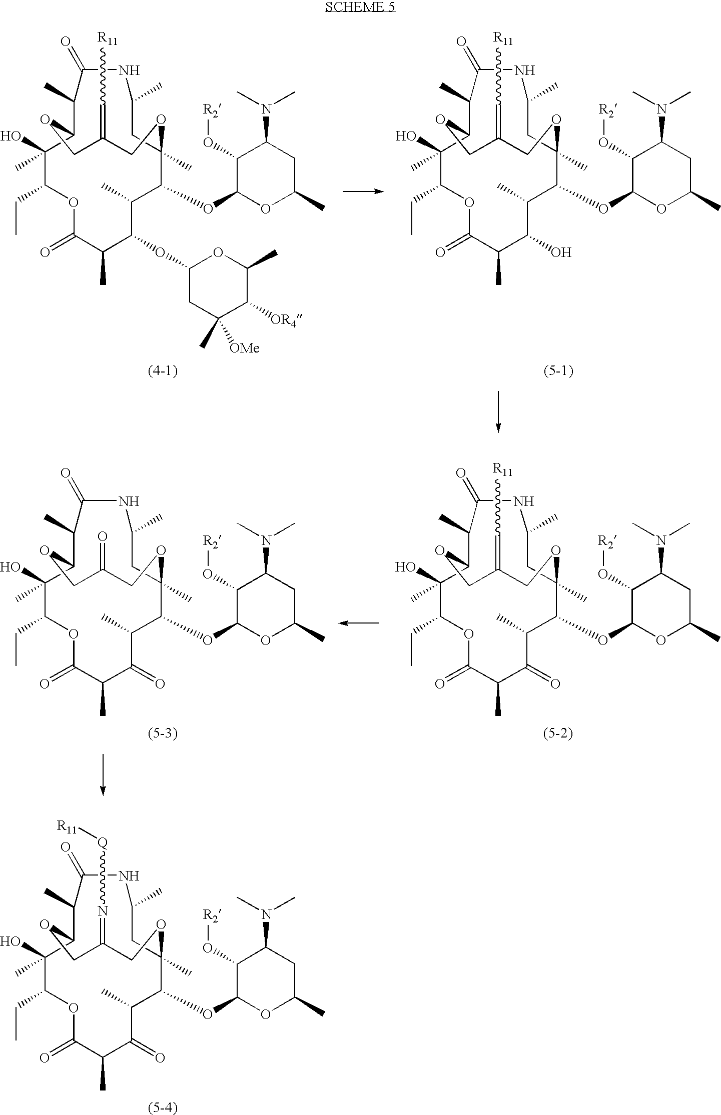

Another process of the invention involves the removal of the cladinose moiety of the compounds of Formula (4-1). The cladinose moiety of the macrolide compound (4-1) is removed by mild acid hydrolysis or by enzymatic hydrolysis to afford compounds of Formula (5-1) in Scheme 5. Representative acids include, but are not limited to, dilute hydrochloric acid, sulfuric acid, perchloric acid, chloroacetic acid, dichloroacetic acid or trifluoroacetic acid. Suitable solvents for the reaction include, but are not limited to, methanol, ethanol, isopropanol, butanol, water, and mixture there of. Reaction times are typically from about 0.5 to about 24 hours. The reaction temperature is preferably from about 0 to about 80° C.

Conversion of compounds of Formula (5-1) to compounds of Formula (5-2) can be accomplished by oxidation of the 3-hydroxy group to a 3-oxo group using Dess-Martin periodinane (for further details concerning the Dess-Martin oxidation see D. B. Dess, J. C. Martin, J. Org. Chem. 48, 4155 (1983)), a Corey-Kim reaction with N-chlorosuccinimide-dimethylsulfide (for further details concerning the Corey-Kim oxidation reaction see E. J. Corey, C. U. Kim, J. Am. Chem. Soc. 94, 7586 (1972)), or a Moffat oxidation with a carbodiimide-DMSO complex in the presence of pyridinium trifluoroacetate, TPAP, PDC, and the like (for further details concerning the Moffat oxidation see J. G. Moffatt, “Sulfoxide-Carbodiimide and Related Oxidations” in Oxidation vol. 2, R. L. Augustine, D. J. Trecker, Eds. (Dekker, New York, 1971) pp 1-64; T. T. Tidwell, Org. React. 39, 297-572 passim (1990); and T. V. Lee, Comp. Org. Syn. 7, 291-303 passim (1991)). In a preferred embodiment, compounds of Formula (5-1) can be treated with Dess-Martin periodinane in dichloromethane at a temperature of from about 0° C. to about 25° C. for about 0.5 to about 4 hours to produce compounds of Formula (5-2).

Conversion of alkenes (5-2) into ketones (5-3) can be accomplished as described previously. The ketone of Formula (5-3) can be converted into an oxime of Formula (5-4) as described previously.

Scheme 6 describes the process by which azalide (3-3) is transformed into the oxime (6-5) using procedures described previously.

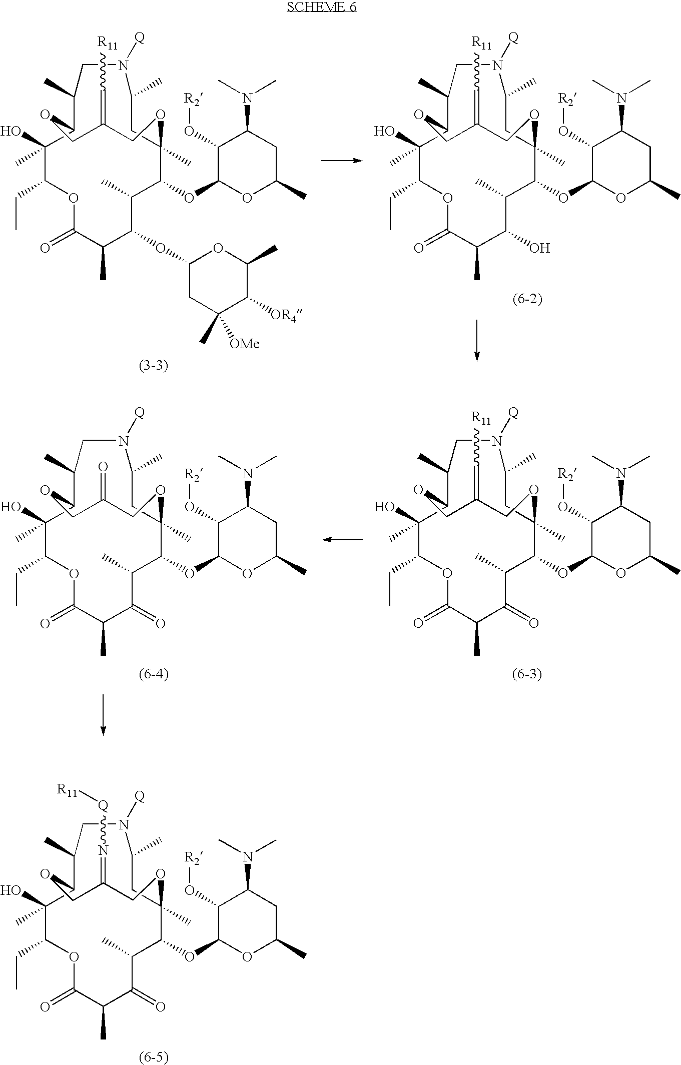

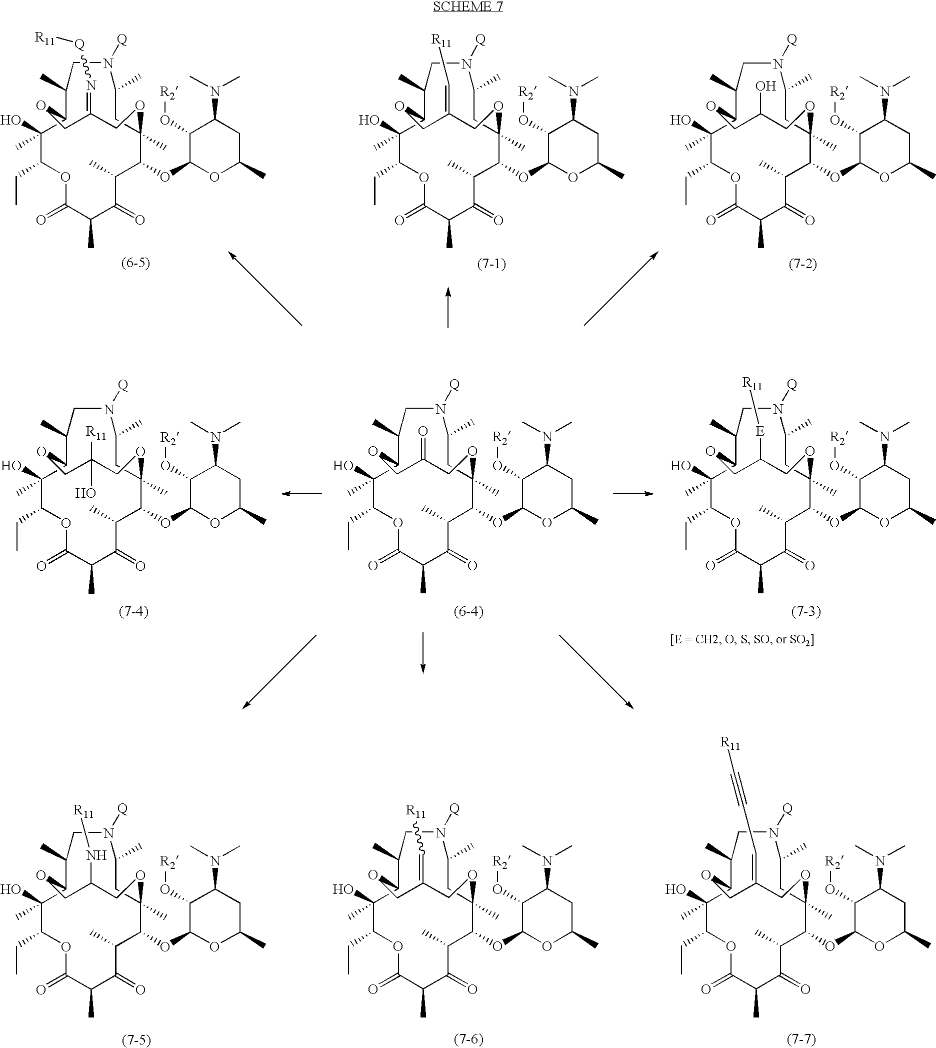

Compounds according to the invention of the Formula (6-4) can be further functionalized in a variety of ways. Scheme 7 details a procedure for the conversion of the ketone of formula (6-4) into an oxime of Formula (7-1). The ketone of Formula (6-4) can be further utilized by conversion into the amine of Formula (7-5) via a reductive amination. Reductive anination can be achieved by treating the ketone with an amine in the presence of a reducing agent to obtain the product amine (7-5). The reaction can be carried out either with or without added acid. Examples of acids that are commonly used include, but are not limited to, hydrochloric acid, phosphoric acid, sulfuric acid, acetic acid, and the like. Reducing agents that can effect reductive amination include, but are not limited to, hydrogen and a catalyst, zinc and hydrochloric acid, sodium cyanoborohydride, sodium borohydride, iron pentacarbonyl, and alcoholic potassium hydroxide. Alcoholic solvents are typically used. The reductive amination preferably employs sodium cyanoborohydride in methanol with added acetic acid.

Yet another means by which to functionalize ketones of Formula (6-4) is via addition of Grignard reagents to form alcohols of Formula (7-4). The requisite Grignard reagents are readily available via the reaction of a variety of alkyl or aryl halides with magnesium under standard conditions (see B. S. Fumiss, A. J. Hannaford, P. W. G. Smith, A. R. Tatchell, Vogel's Textbook of Practical Organic Chemistry, 5^{th} ed., Longman, 1989). The addition is performed in an inert solvent, generally at low temperatures. Suitable solvents include, but are not limited to, tetrahydrofuran, diethylether, 1,4-dioxane, 1,2-dimethoxyethane, and hexanes. Preferably the solvent is tetrahydrofuran or diethylether. Preferably the reaction is run at a temperature of from about −78° C. to about 0° C.

In a similar way, reaction with other organometallic reagents gives rise to alcohols of Formula (7-4). Examples of useful organometallic reagents include, but are not limited to, organo-aluminum, organo-lithium, organo-cerium, organo-zinc, organo-thallium, and organo-boron reagents. A more thorough discussion of organometallic reagents can be found in B. S. Fumiss, A. J. Hannaford, P. W. G Smith, A. R. Tatchell, Vogel's Textbook of Practical Organic Chemistry. 5^{th} ed., Longman, 1989.

Furthermore, alcohols of type (7-2) can be prepared by reduction of the corresponding ketone of Formula (6-4) under a variety of conditions (see Hudlicky, M. Reductions in Organic Chemistry, Ellis Horwood Limited: Chichester, 1984). The alcohols thus derived can be further modified to give compounds of Formula (7-3). A process to generate compounds of Formula (7-3) includes, but is not limited to, alkylation of the alcohol with an electrophile or conversion of the alcohol into a leaving group, such as a triflate, tosylate, phosphonate, halide, or the like, followed by displacement with a heteroatom nucleophile (e.g. an amine, alkoxide, sulfide or the like).

It will be appreciated by one skilled in the art that ketones of Formula (6-4) can be transformed into alkenes of Formula (7-1) and (7-6) via the Wittig reaction with the appropriate phosphonium salt in the presence of a base, see (a) Burke, Tetrahedron Lett., 1987, 41434146, (b) Rathke and Nowak, J. Org. Chem., 1985, 2624-2626, (c) Maryanoff and Reitz, Chem. Rev, 1989, 863-927. Furthermore, vinyl halides of Formula (7-6) can be functionalized by Sonogashira coupling with alkynes in the presence of a palladium catalyst, a copper halide and an amine base to give compounds of Formula (7-7) (see (a) Sonogashira, Comprehensive Organic Synthesis, Volume 3, Chapters 2, 4; (b) Sonogashira, Synthesis 1977, 777). In a similar manner, alkenes of Formula (7-1) can be obtained from vinyl halides (7-6) via Suzuki-cross coupling with organoboron reagents in the presence of a palladium catalyst and a base, or via Stille cross coupling with organostannanes in the presence of a palladium catalyst (see (a) Suzuki, J. Organomet. Chem. 1999, 576, 147-168, (b) Stille, Angew Chem. Int. Ed. Engl., 1986, 508-524 (c) Farina, J. Am. Chem. Soc., 1991, 9585-9595).

It will be appreciated by one skilled in the art that the unsaturated compounds represented by compounds (7-1) and (7-7) can be reduced to form the corresponding saturated compound (see Hudlicky, M., Reductions in Organic Chemistry, Ellis Horwood Limited: Chichester, 1984).

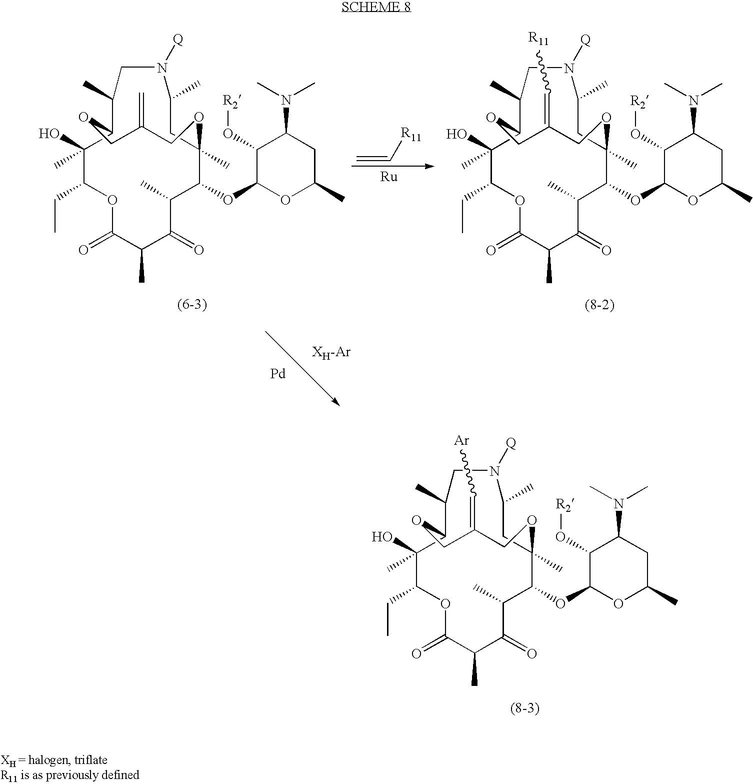

Compounds of the invention according to Formula (6-3), where R₁₁ is hydrogen, are also capable of further functionalization to generate compounds of the present invention. As depicted in Scheme 8, alkene (6-3) can be treated with an aryl halide or aryl triflate in the presence of a palladium catalyst [Pd(O) or Pd(II)] to provide compound (8-3): (See (a) Heck, Palladium Reagents in Organic Synthesis, Academic Press: New York, 1985, Chapter 1; (b) Sonogashira, Comprehensive Organic Synthesis, Volume 3, Chapters 2, 4; (c) Sonogashira, Synthesis 1977, 777). Under the Heck coupling conditions, regioisomers and stereoisomers of the double bond are possible. Alternatively, compound (6-3) can undergo a cross metathesis reaction with vinylaromatic derivatives using ruthenium catalysts to give compounds of Formula (8-2) (see (a) J. Org. Chem. 2000, 65, 2204-2207; (b) Reviews: Synlett. 1999, 2, 267; (c) Reviews: Ivin, K. J.; Mol, J. C., Olefin Metathesis and Metathesis Polymerization, 2^{nd} ed., Academic Press: New York, 1997; (d) J. Org. Chem. 1999, 64, 4798-4816; (e) Angew. Chem., Int Ed. Engl. 1997, 36, 2036-2056; (f) Tetrahedron 1998, 54, 4413-4450).

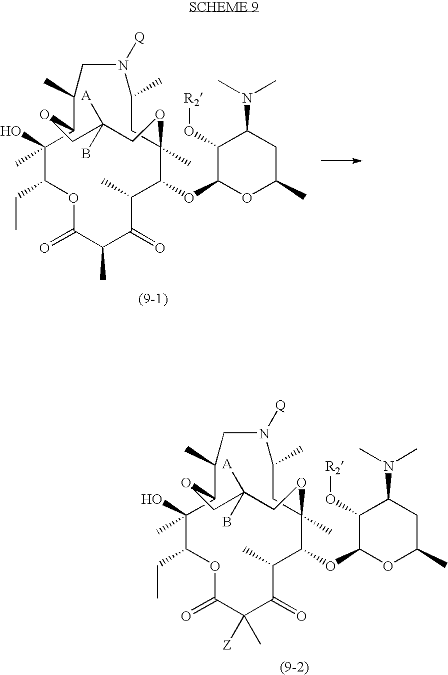

Scheme 9 illustrates the procedure by which compounds of Formula (9-1), wherein A, B, Q, and R₂′ are as previously defined, may be converted to compounds of Formula (9-2), wherein A, B, Q, Z, and R₂′ are as previously defined, by treatment with a halogenating reagent. This reagent acts to replace a hydrogen atom with a halogen atom at the C-2 position of the ketolide (i.e., Z=halogen in 9-2). Various halogenating reagents are suitable for this procedure. Suitable fluorinating reagents include, but are not limited to, N-fluorobenzenesulfonimide in the presence of base, 10% F₂ in formic acid, 3,5-dichloro-1-fluoropyridinium tetrafluoroborate, 3,5-dichloro-1-fluoropyridinium triflate, (CF₃SO₂)₂NF, N-fluoro-N-methyl-p-toluenesulfonamide in the presence of base, N-fluoropyridinium triflate, and N-fluoroperfluoropiperidine in the presence of base. Suitable chlorinating reagents include, but are not limited to, hexachloroethane in the presence of base, CF₃CF₂CH₂ICl₂, SO₂Cl₂, SOCl₂, CF₃SO₂Cl in the presence Cl₂, and NaOCl in the presence of acetic acid. Suitable brominating reagents include, but are not limited to, Br₂.pyridine.HBr, Br₂/acetic acid, N-bromosuccinimide in the presence of base, LDA/BrCH₂CH₂Br, and LDA/CBr₄. A suitable iodinating reagents include, but are not limited to, N-iodosuccininide in the presence of base and I₂.

Suitable bases for the halogenating reactions requiring them are compounds such as alkali metal hydrides, such as NaH and KH, or amine bases, such as LDA or triethylamine, for example. Different reagents may require different types of base, but this is well known within the art.

A preferred halogenating reagent is N-fluorobenzenesulfonimide in the presence of sodium hydride.

Suitable solvents are dimethylformamide, dimethyl sulfoxide, pyrrolidinone and the like.

It will be appreciated by one skilled in the art that all ketolide compounds delineated herein may be halogenated at the 2-carbon if so desired.

All references cited herein, whether in print, electronic, computer readable storage media or other form, are expressly incorporated by reference in their entireties, including but not limited to, abstracts, articles, journals, publications, texts, treatises, internet web sites, databases, patents, and patent publications.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Example 1

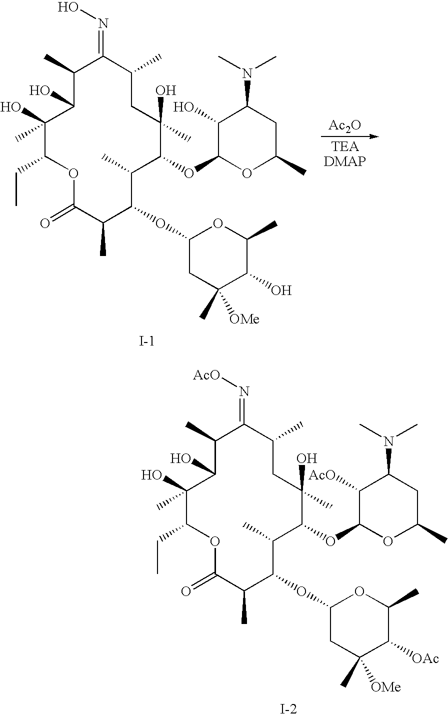

### Step 1a

Acetic anhydride (3.33 mL, 35.29 mmol), triethylamine (5.25 mL, 37.63 mmol) and 4-(dimethylamino)pyridine (144 mg, 1.18 mmol) were added to a solution of compound I-1 (8.8 g, 11.76 mmol) in anhydrous tetrahydrofuran (47 mL). The mixture was stirred at room temperature for 18 hours and was partitioned between ethyl acetate (500 mL) and 5% aqueous sodium bicarbonate (500 mL). The ethyl acetate layer was washed with additional 5% aqueous bicarbonate (4×500 mL), brine (200 mL), dried with magnesium sulfate, filtered and evaporated to give compound I-2 as a foam (9.48 grams) after freeze-drying from benzene.

MS m/e (M+H)=875.39.

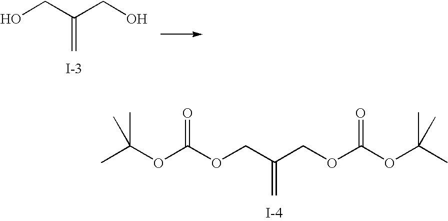

### Step 1b

SN sodium hydroxide (34 mL, 172 mmol) was added dropwise to a solution of 2-methylene-1,3-propanediol 1-3 (2 mL, 24.51 mmol) and tetrabutylammonium hydrogensulfate (1.39 g, 4.09 mmol) in methylene chloride (60 mL). The mixture was stirred at room temperature for 18 hours and was added to 5% aqueous sodium bicarbonate (100 mL). The organic phase was washed with additional 5% aqueous sodium bicarbonate (2×100 mL), brine (100 mL), dried with magnesium sulfate, filtered and evaporated to give I-4 (5.87 g).

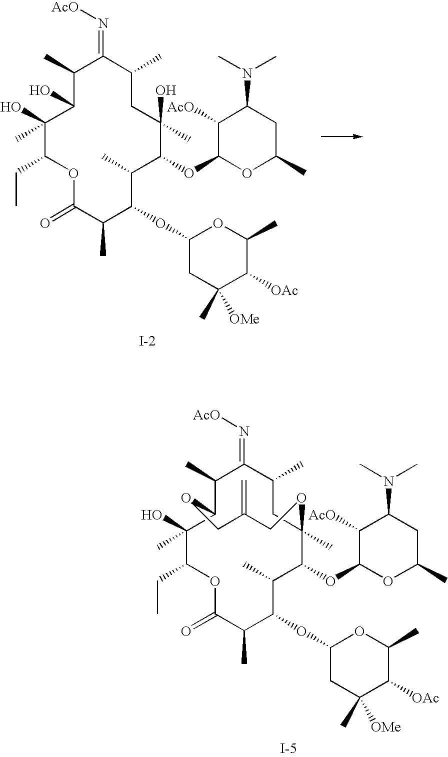

### Step 1c

A solution of I-2 (8.7 g, 0.01 mmol) and I-4 (3.6 g, 0.0125 mmol) in anhydrous toluene (100 mL) was evaporated under vacuum. The residue was redissolved in anhydrous toluene (100 mL) and evaporated. Palladium II acetate (157 mg, 0.0007 mmol) and triphenylphosphine (1.05 g, 0.004 mmol) were added and the mixture was placed under high vacuum for 10 minutes. Anhydrous tetrahydrofuran (100 mL) was added and the reaction was repeatedly placed under vacuum and blanketed with nitrogen (5×). The resulting mixture was placed in a 70° C. oil bath for 7 hours under an atmosphere of nitrogen. After cooling to room temperature the solvent was evaporated. The solid was placed on a 6.5×130 cm silica 60 column. The column was eluted with 1:1 acetone/hexanes collecting 25 mL fractions. After a 150 mL forerun, fractions 24-44 were combined and evaporated to give the title compound I-5 (3.87 g).

MS m/e (M+H)=927.54. IR (CM⁻¹) 2976, 1741, 1454, 1370, 1236, 1173, 1049. ¹³C NMR (CDCl₃): δ (ppm) 175.9, 174.7, 170.7, 170.1, 168.6, 141.8, 123.0, 100.5, 96.1, 79.6, 78.7, 78.4, 78.1, 76.9, 74.4, 73.0, 71.0, 67.9, 65.3, 63.6, 63.4, 49.7, 44.3, 41.5, 41.0, 36.8, 35.1, 35.0, 31.3, 23.5, 21.8, 21.7, 21.4, 21.2, 21.0, 20.9, 20.3, 18.2, 17.3, 14.3, 13.4, 12.9, 8.73.

### Example 2

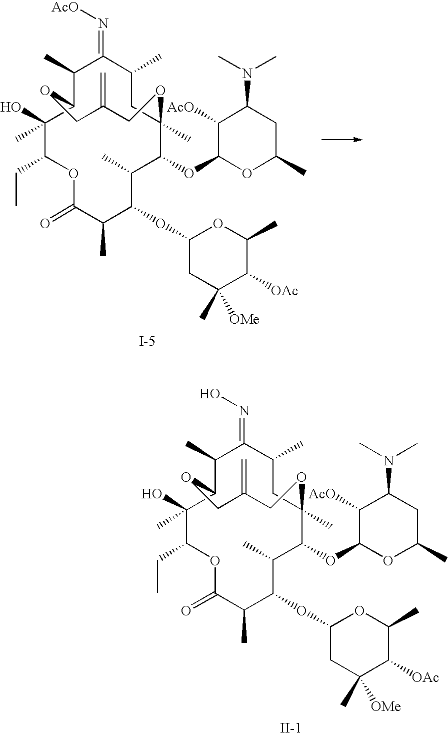

### Step 2a

A solution of lithium hydroxide monohydrate (340 mg, 8.1 mmol) in water (8.1 mL) was added to a solution of I-5 (1.25 g, 1.35 mmol) in a mixture of tetrahydrofuran (9.3 mL) and isopropyl alcohol (9.3) in an ice bath. After stirring rapidly for 40 minute, ethyl acetate (100 mL) and 5% aqueous sodium bicarbonate (50 mL) were added, the aqueous layer was re-extracted with additional ethyl acetate (3×50 mL), the combined organic layers were dried with magnesium sulfate, filtered and evaporated to give compound II-1 (1.25 g).

MS m/e (M+H)=885.54.

### Example 3

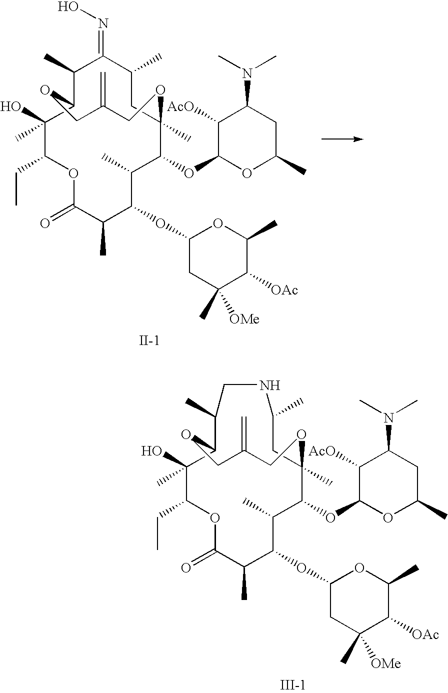

### Step 3a

A solution of II-1 (1.25 grams) was dissolved in anhydrous toluene (30 mL) and evaporated under vacuum to a foam. This process was repeated twice. The resulting solid (1.19 g, 1.35 mmol) was dissolved in methylene chloride (13.6 mL) and cooled in a −30° C. dry ice/acetonitrile bath. Addition of triethylamine (0.455 mL, 3.24 mmol) was followed by the addition of toluene sulfonic anhydride (0.529 g, 1.62 mmol). The bath temperature was allowed to increase to −20° C. over 30 minutes. Addition of anhydrous methanol (13.6 mL) was followed by a slow increase in bath temperature to 0° over 30 minutes. Addition of sodium borohydride (0.255 g, 6.75 mmol) was followed by a slow increase in bath temperature to 7° C. over 3 hours. A 5% aqueous Tris solution (50 mL) was added to the mixture which was stirred at room temperature for 1 hour. The aqueous layer was extracted with ethyl acetate (1×100 mL, 3×75 mL), the combined layers were washed with brine (50 mL), dried with magnesium sulfate, filtered and evaporated to give compound III-1 (1.25 g).

MS m/e (M+H)=871.6. ¹³C NMR (CDCl₃): δ (ppm) 177.4, 170.5, 169.8, 141.7, 120.8, 100.1, 94.4, 80.6, 78.7, 78.1, 76.4, 75.7, 72.9, 71.8, 68.1, 67.3, 66.3, 63.2, 62.9, 54.3, 51.2, 49.3, 44.7, 44.0, 40.7, 34.8, 34.1, 31.3, 23.0, 22.8, 21.6, 21.5, 21.3, 20.9, 18.1, 17.7, 13.9, 13.2, 12.7, 12.6, 8.63.

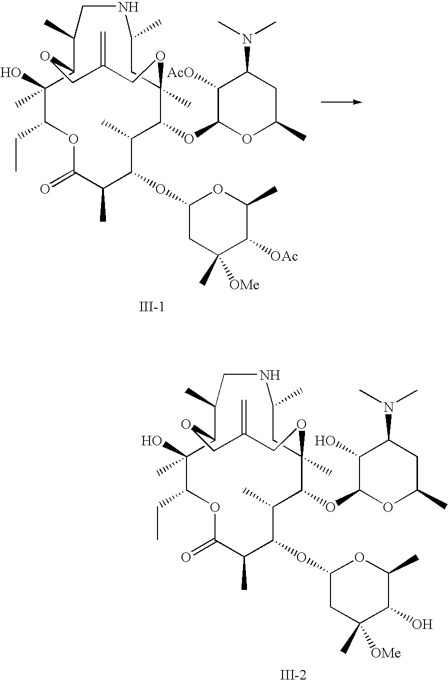

### Step 3b

A solution of III-1 (290 mg, 0.333 mmol) and potassium carbonate (290 mg, 2.10 mmol) in methanol (5.0 mL) was stirred at room temperature for 24 hours. The mixture was partitioned between ethyl acetate (30 mL) and brine (10 mL). The aqueous layer was extracted with ethyl acetate (2×10 mL) and the combined organic layers were dried with magnesium sulfate, filtered and evaporated to give III-2 as a solid (260 mg).

MS m/e (M+H)=787.77.

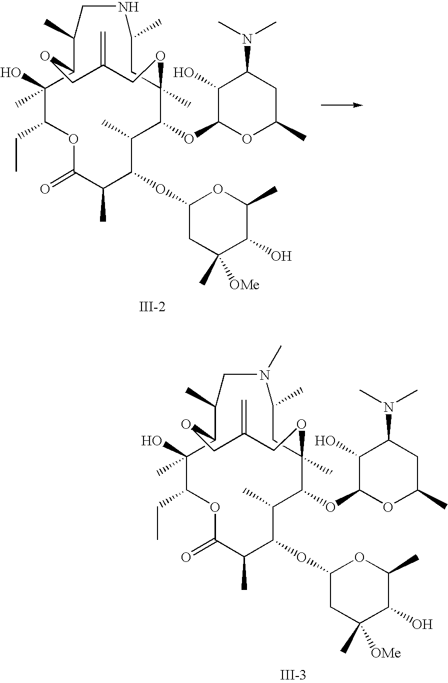

### Step 3c

Sodium cyanoborohydride (0.165 g, 2.6 mmol) was added to a solution of III-2 (0.26 g, 0.33 mmol), 37% aqueous formaldehyde (0.315 mL, 3.89 mmol) and acetic acid (0.315 mL, 5.3 mmol) in methanol (31.5 mL) at room temperature. After 2 hours, a 5% aqueous tris solution (50 mL) was added and the solution was stirred for an additional 2 hours. The aqueous layer was extracted with ethyl acetate (1×100 mL, 1×50 mL). The combined organic layers were washed with brine (100 mL), dried with magnesium sulfate, filtered and evaporated to a solid (0.3 g). The solid was placed on a 2.75×15 cm silica gel 60 column. The column was eluted with 1:1 acetone/hexanes collecting 8 mL fractions. Fractions 19-32 were combined and evaporated to give the title compound III-3 (0.18 g).

MS m/e (M+H)=801.64.

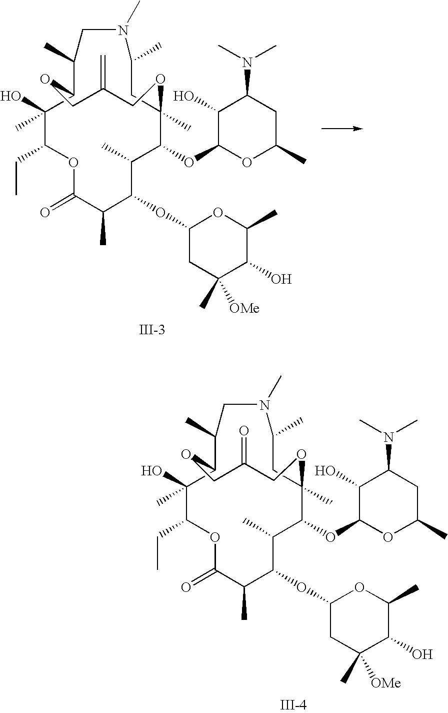

### Step 3d

A solution of III-3 (60 mg, 0.075 mmol) and camphorsulfonic acid (34.8 mg, 0.15 mmol) in methanol (3.0 mL) was cooled in a dry ice/acetone bath. Ozone was bubbled into the solution for 10 minutes after a blue color was first observed. Oxygen was bubbled through the solution until the blue color dissipated, dimethylsulfide (0.055 mL, 0.75 mmol) was added and the reaction was brought to room temperature. After 3 hours, the solvent was evaporated and the residue was loaded onto a silica column (1.25×9 cm) which was eluted with a mixture of 60:10:1 methylene chloride/methanol/concentrated ammonium hydroxide. The product fractions were combined and evaporated to give III-4 as a solid (0.06 g).

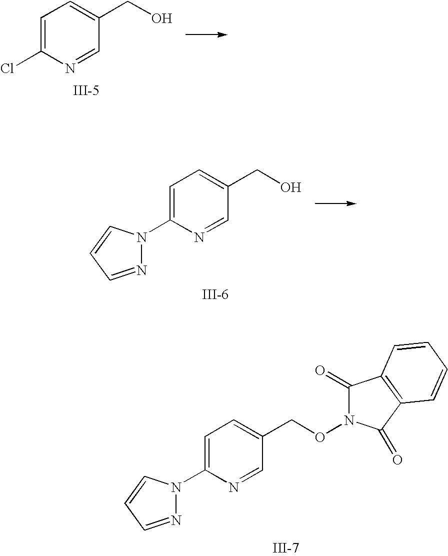

### Step 3e

A suspension of 2-chloro-5-hydroxymethylpyridine III-5 (144 mg, 1 mmol), pyrazole (340 mg, 5 mmol), and potassium carbonate (828 mg, 6 mmol) in dimethylacetamide (1 mL) was stirred in a 140° C. oil bath for 23 hours. The oil bath temperature was increased to 150° C. and the reaction was stirred an additional 8 hours. After cooling to room temperature, ethyl acetate (20 mL) and water (20 mL) were added. The organic layer was washed with water (20 mL) and brine (10 mL), dried with magnesium sulfate, filtered and evaporated to an oil (144 mg). The oil was placed on 3×500 micron reverse-phase preparative silica plates which were developed with 25% acetonitrile/water to give III-6 (30 mg).

¹H NMR (CDCl₃): δ 1.85 (t, 1H); 4.76 (d, 2%); 6.48 (d, 1H); 7.74 (d, 1H); 7.85 (dd, 1H); 7.98 (d, 1H); 8.39 (d, 1H); 8.57 (d, 1H).

### Step 3f

Diethyl azodicarboxylate (0.054 mL, 0.343 mmol) was added dropwise to a solution of III-6 (40 mg, 0.229 mmol), and triphenylphosphine (90 mg, 0.343 mmol) in anhydrous tetrahydrofuran (0.92 mL) at room temperature. After 5 minutes, N-hydroxyphthalimide (56 mg, 0.343 mmol) was added and the light red colored solution was stirred for 18 hours. The solvent was evaporated, the residue was placed on 2×100 micron preparative silica gel plates and developed with 20% ethyl acetate/methylene chloride to give III-7 as a solid (31.3 mg).

¹H NMR (CDCl₃): 5.26 (s, 2H); 6.47 (m, 1H); 7.75 (m, 3H); 7.83 (m, 2H); 8.03 (d, 1H); 8.08 (dd, 1H); 8.50 (d, 1H); 8.57 (d, 1H).

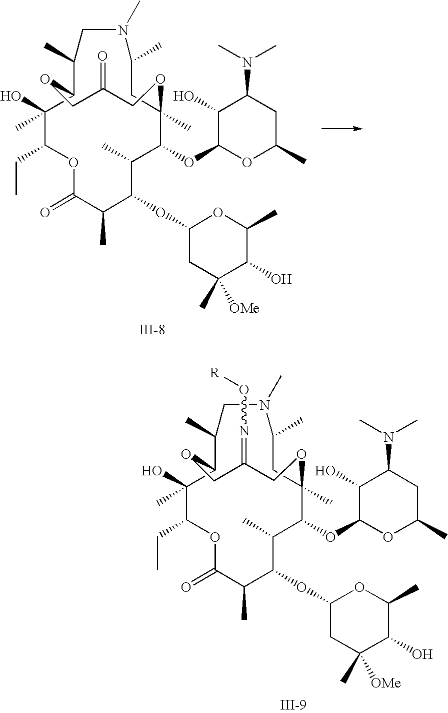

### Step 3g: R=phenyl

A solution of III-8 (10.2 mg, 0.013 mmol), O-phenylhydroxylamine hydrochloride (3.7 mg, 0.025 mmol) and pyridine (0.0042 mL, 0.0524 mmol) in ethanol (0.26 mL) was stirred at room temperature for 18 hours and then heated in a 40° C. oil bath for 1.5 hours. The solvent was evaporated and the residue was placed on a 1 mL silica column which was eluted with a mixture of 60:10:1 methylene chloride/methanol/concentrated ammonium hydroxide to give III-9 (9.0 mg) as a mixture of a major and minor oxime isomers. The oxime isomers could be separated by preparative silica plate chromatography (120/10/1 methylene chloride/methanol/concentrated ammonium hydroxide, developed 2×).

MS m/e (M+H)=894.93.

### Step 3h: R=benzyl

A solution of III-8 (13 mg, 0.016 mmol), O-benzylhydroxylamine hydrochloride (5.3 mg, 0.033 mmol) and pyridine (0.0052 mL, 0.064 mmol) in ethanol (0.32 mL) was stirred at room temperature for 2.5 hours. The solvent was evaporated and the residue was placed on a 1 mL silica column which was eluted with a mixture of 60:10:1 nethylene chloride/methanol/concentrated ammonium hydroxide to give III-9 (11.3 mg) as a mixture of a major and minor oxime isomers. The oxime isomers could be separated by preparative silica plate chromatography (120/10/1 methylene chloride/methanol/concentrated ammonium hydroxide, developed 2×).

MS m/e (M+H)=908.91.

### Step 31: R=phenethyl

Hydrazine hydrate (5.3 uL, 0.109 mmol) was added to a solution of N-phthaloyl-O-phenethyl-hydroxylamine (39 mg, 0.146 mmol) in ethanol (1.0 mL) and the mixture was heated in a 60° C. oil bath 75 minutes. After cooling to room temperature, acetic acid (3 uL, 0.057 mmol) and III-8 (15.0 mg, 0.019 mmol) were added and the mixture was stirred at room temperature for 18 hours. The solvent was evaporated and the residue was placed on 2×500 u preparative tlc plates which were developed with a mixture of 120:10:1 methylene chloride/methanol/concentrated ammoniun hydroxide to give III-9 (10.0 mg) as an approximately equal mixture of a oxime isomers. The oxime isomers were separated by an additional preparative silica plate chromatography (120/10/1 methylene chloride/methanol/concentrated ammonium hydroxide).

MS m/e (M+H)=922.89.

### Step 3j: R=[2-(pyrazol-1-yl)pyrid-5-yl]methyl

Hydrazine hydrate (1.4 uL, 0.029 mmol) was added to a solution of III-7 (12 mg, 0.038 mmol) in ethanol (0.3 mL) and the mixture was heated in a 40° C. oil bath 3 hours. After cooling to room temperature, acetic acid (3.3 uL, 0.057 mmol) and III-8 (15.0 mg, 0.019 mmol) were added and the mixture was stirred in a 40° oil bath for 18 hours. The solvent was evaporated and the residue was placed on 2×500 u preparative thin layer chromatography plates which were developed with a mixture of 120:10:1 methylene chloride/methanol/concentrated ammonium hydroxide to give III-9 as oxime isomer 1 (3.2 mg) and oxime isomer 2 (4.1 mg).

MS m/e (M+H)=975.69.

### Example 4

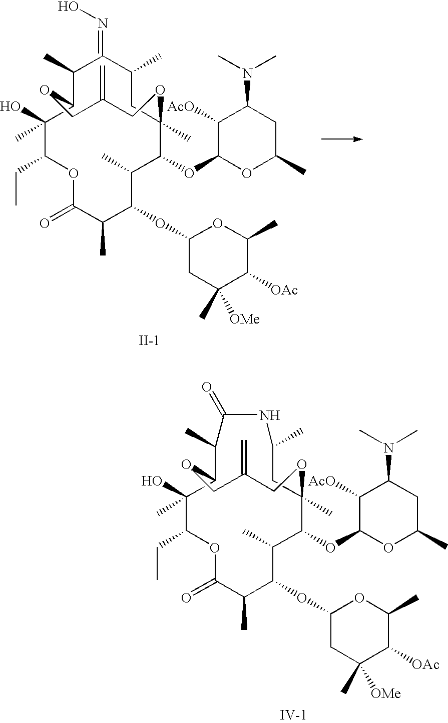

### Step 4a

A solution of toluenesulfonyl chloride (122 mg, 0.64 mmol) in acetone (0.5 mL) was added to an ice cooled suspension of II-1 (270 mg, 0.31 mmol) and sodium bicarbonate (108 mg, 1.28 mmol) in a mixture of acetone (1.5 ml) and water (1.5 mL). After 30 minutes, the reaction was removed from the ice bath and allowed to warm to room temperature. After an additional hour, the reaction was judged to be essentially complete and was stored in the freezer for 66 hours. After warming to room temperature, ethyl acetate (300 mL) and brine were added (100 mL), the organic layer was dried with magnesium sulfate, filtered and evaporated to give compound IV-1 (267 mg).

MS m/e (M+H)=885.88.

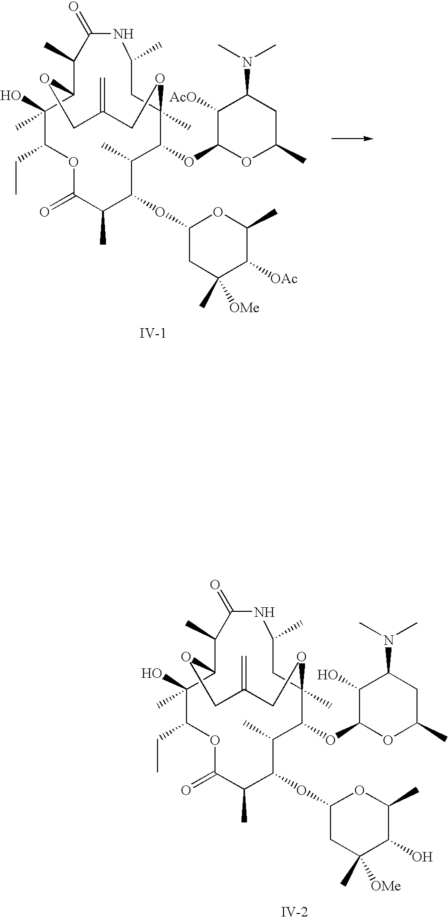

### Step 4b

A solution of IV-1 (SOng, 0.057 mmol) and potassium carbonate (47 mg, 0.339 mmol) in methanol (1.0 mL) was stirred at room temperature for 26 hours. The mixture was partitioned between ethyl acetate (20 mL) and 5% aqueous bicarbonate (20 mL). The organic layer was washed with brine (10 mL), dried with magnesium sulfate, filtered and evaporated to give IV-2 as a solid (35 mg).

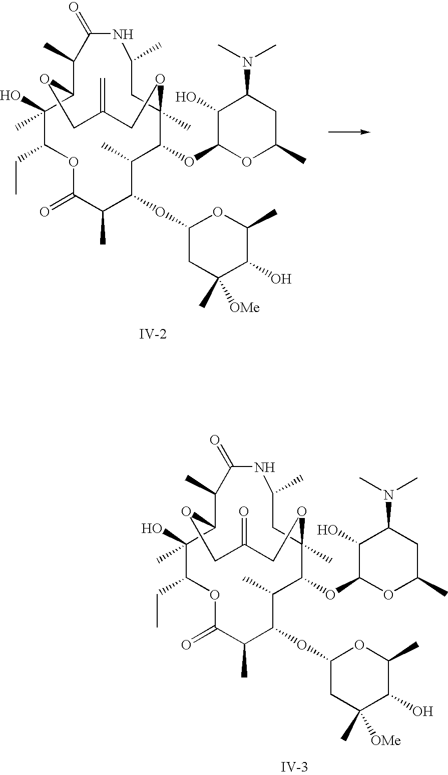

### Step 4c

A solution of IV-2 (25 mg, 0.031 mmol) and camphorsulfonic acid (7.2 mg, 0.031 mmol) in methanol (1.25 mL) was cooled in a dry ice/acetone bath. Ozone was bubbled into the solution for 10 minutes after a blue color was first observed. Oxygen was bubbled through the solution until the blue color dissipated. Dimethylsulfide (0.023 mL, 0.31 mmol) was added and the reaction was brought to room temperature. After 3 hours, the solvent was evaporated and the residue was loaded onto a 1 mL silica column which was eluted with a mixture of 60:10:1 methylene chloride/methanol/concentrated ammonium hydroxide. The product fractions were combined and evaporated to give IV-3 as a solid (23.3 mg).

MS m/e (M+H)=803.76.

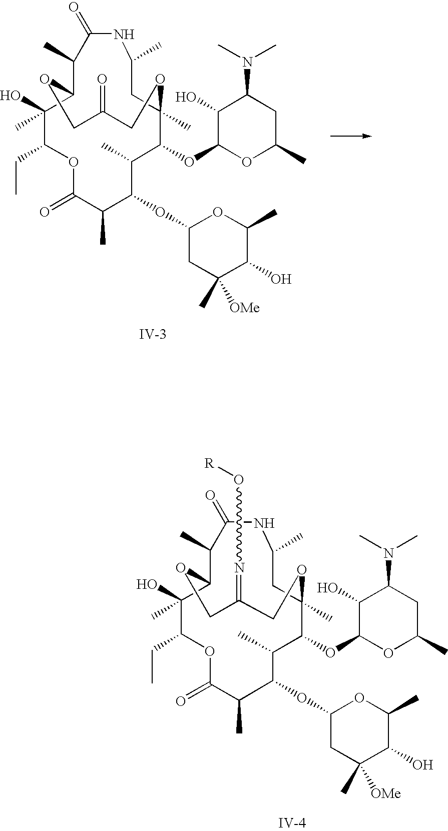

### Step 4d: R=benzyl

A solution of IV-3 (15 mg, 0.019 mmol), O-benzylhydroxylamine hydrochloride (6.0 mg, 0.037 mmol) and pyridine (0.006 mL, 0.076 mmol) in ethanol (0.35 mL) was stirred at room temperature for 6 hours. The solvent was evaporated and the residue was placed on a 1×500 u preparative silica plates which was eluted with a mixture of 60:10:1 methylene chloride/methanol/concentrated ammonium hydroxide to give IV-4 (12 mg) as a mixture of a major and minor oxime isomers. The oxime isomers could be separated by reverse phase HPLC.

MS m/e (M+H)=908.70.

### Example 5

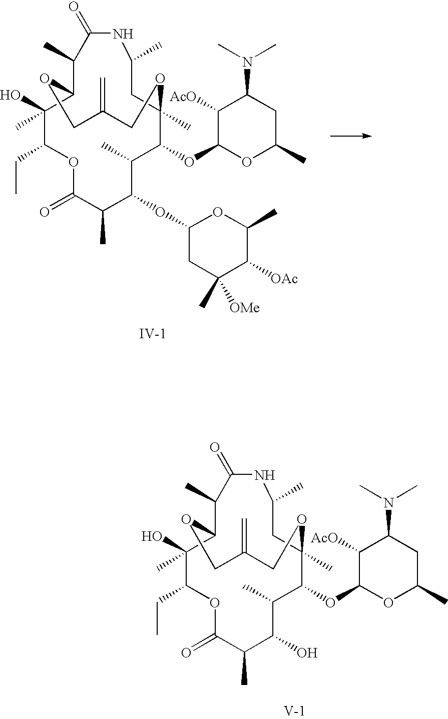

### Step 5a

Acetyl chloride (0.1 mL, 1.4 mmol) was added dropwise to methanol (2.0 mL). After 15 minutes, IV-1 (120 mg, 0.136 mmol) was added and the solution was placed in a 60° C. oil bath for 6.5 hours. After cooling to room temperature, the solvent was evaporated and the residue was placed on a 2.75×8 cm silica column which was eluted with a mixture of 60:10:1 methylene chloride/methanol/concentrated ammonium hydroxide to give V-1 (86.5 mg).

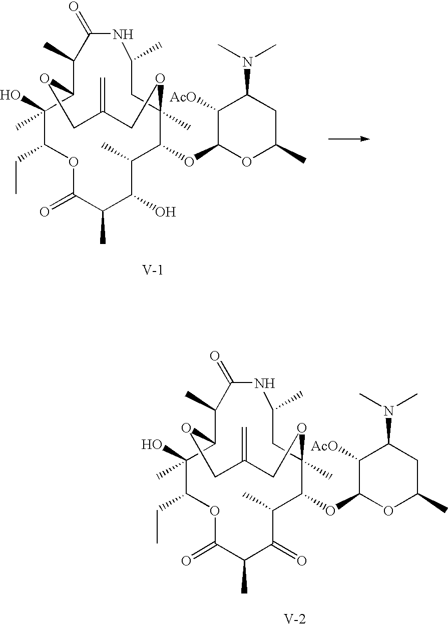

### Step 5b

Dimethyl sulfoxide (0.09 mL, 1.26 mmol), 1-[3-(dimethylamino)propyl]-3-ethylcarbodiimide hydrochloride (48 mg, 0.252 mmol) and pyridinium trifluoracetate (48 mg, 0.252 mmol) were added to a solution of V-1 (86.5 mg, 0.126 mmol) in methylene chloride (0.5 mL). The solution was stirred for 2.5 hours and additional carbodiimide (24 mg, 0.13 mmol) and pyridinium trifluoracetate (24 mg, 0.13 mmol) were added. After 60 minutes, the reaction mixture was evaporated and placed onto a 2.75×8 cm silica column which was eluted with a mixture of 60; 10:1 methylene chloride/methanol/concentrated ammonium hydroxide to give V-2 (80 mg).

MS m/e (M+H)=683.82.

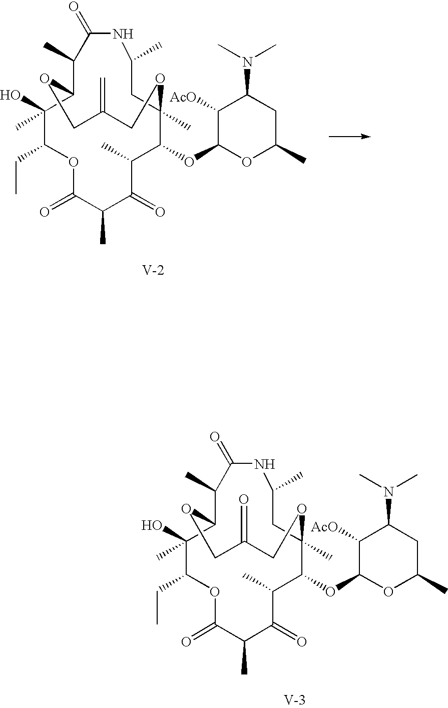

### Step 5c

A solution of V-2 (30 mg, 0.044 mmol) and camphorsulfonic acid (10.2 mg, 0.044 mmol) in methanol (2.5 mL) was cooled in a dry ice) acetone bath. Ozone was bubbled into the solution for 10 minutes after a blue color was first observed. Oxygen was bubbled through the solution until the blue color dissipated. Dimethylsulfide (0.032 mL, 0.44 mmol) was added and the reaction was brought to room temperature. After 24 hours, the solvent was evaporated and the residue was loaded onto a lrnL silica column which was eluted with a mixture of 60:10:1 methylene chloride/methanol/concentrated ammoniun hydroxide. The product fractions were combined and evaporated to give V-3 as a solid (29 mg) after freezedrying from benzene.

MS m/e (M+H)=685.45.

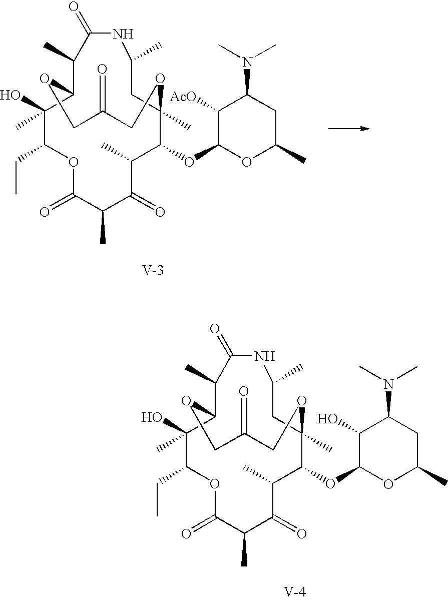

### Step 5d

A solution of V-3 (39 mg) in methanol (1.0 mL) was stirred at room temperature for 18 hours. The solvent was evaporated to give V-4 (29 mg).

MS m/e (M+H)=675.79 (MeOH ketal).

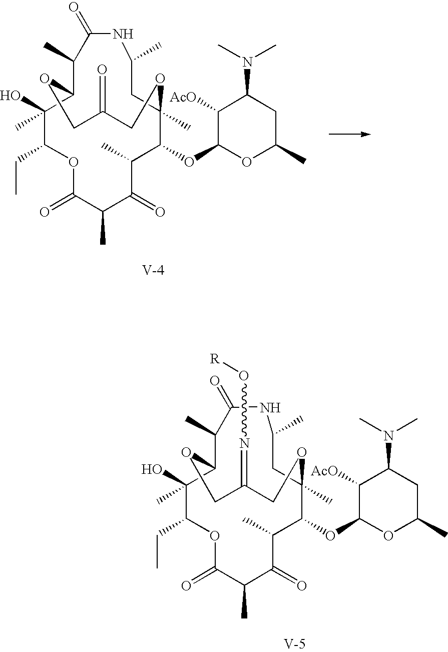

### Step 5e: R=benzyl

A solution of V-4 (10 mg, 0.016 mmol), O-benzylhydroxylamine hydrochloride (5 mg, 0.031 mmol) and pyridine (0.005 mL, 0.062 mmol) in ethanol (0.16 mL) was stirred at room temperature for 1 hour. The solvent was evaporated and the residue was placed on a 1×500 u preparative silica plate which was eluted with a mixture of 60:10:1 methylene chloride/methanol/concentrated ammonium hydroxide to give V-5 (4.6 mg) as a mixture of oxime isomers. The oxime isomers could be separated by preparative silica plate chromatography (120:10:1 methylene chloride/methanol/concentrated ammonium hydroxide).

MS m/e (M+H)=748.80.

### Step 5f: R=[2-(pyrazol-1-yl)pyrid-5-yl]methyl

Hydrazine hydrate (1.2 uL, 0.025 mmol) was added to a solution of III-7 (7.9 mg, 0.025 mmol) in ethanol (0.3 mL) and the mixture was heated in a 40° C. oil bath 3 hours. After cooling to room temperature, acetic acid (4.3 uL, 0.075 mmol) and V-4 (16.0 mg, 0.025 mmol) were added and the mixture was stirred in a 40° C. oil bath for 3.5 hours. The solvent was evaporated and the residue was placed on a 1 mL silica column which was eluted with a mixture of 60:10:1 methylene chloride/methanol/concentrated ammonium hydroxide to give V-5 (18 mg) as a mixture of a major and minor oxime isomers. The oxime isomers could be separated by preparative silica plate chromatography (120:10:1 methylene chloride/methanol/concentrated ammonium hydroxide).

MS m/e (M+H)=815.82.

### Example 6

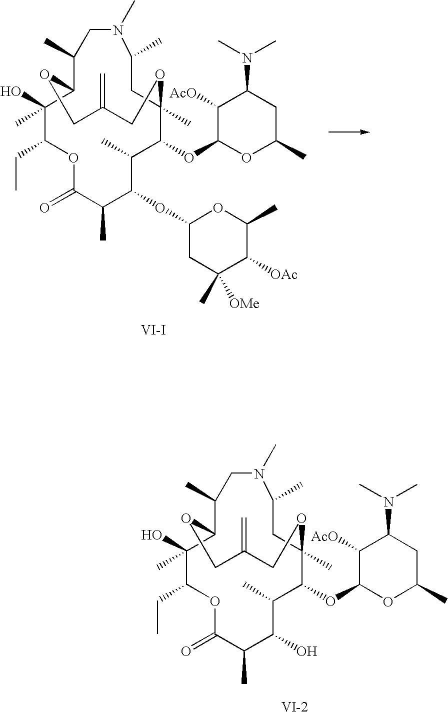

### Step 6a

Acetyl chloride (0.2 mL, 2.8 mmol) was added dropwise to methanol (4.0 mL). After 15 minutes, VI-1 (180 mg, 0.2 mmol) was added and the solution was placed in a 60° C. oil bath for 6 hours. After cooling to room temperature, the solvent was evaporated and the residue was placed on a 1 mL silica column which was eluted with a mixture of 60:10:1 methylene chloride/methanol/concentrated ammonium hydroxide to give VI-2 (15 mg).

MS m/e (M+H)=643.67.

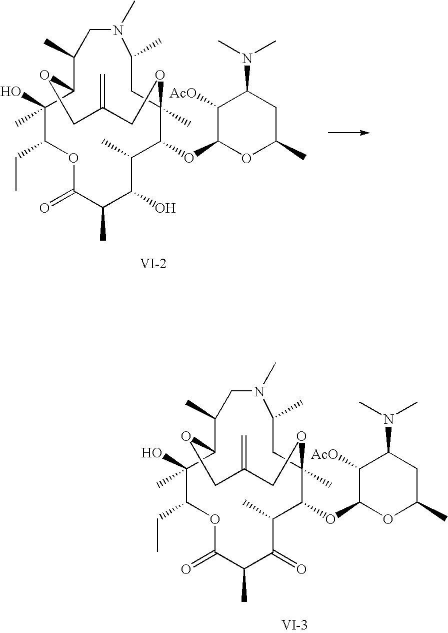

### Step 6b

Dimethyl sulfoxide (0.05 mL, 0.7 mmol), 1-[3-dimethylamino)propyl]-3-ethylcarbodiimide hydrochloride (40.3 mg, 0.21 mmol) and pyridinium trifluoracetate (40.5 mg, 0.21 mmol) were added to a solution of VI-2 (45 mg, 0.07 mmol) in methylene chloride (0.5 mL). The solution was stirred for 30 hours and additional carbodiimide (40.3 mg, 0.21 mmol) and pyridinium trifluoracetate (40.5 mg, 0.21 mmol) were added. After 18 hours, additional carbodiimide (20 mg, 0.11 mmol) was added. After 80 minutes, the reaction mixture was evaporated and placed onto a 2.75×10 cm silica column which was eluted with a mixture of 60:10:1 methylene chloride/methanol/concentrated ammonium hydroxide to give VI-3 (24 mg).

MS m/e (M+H)=641.72.

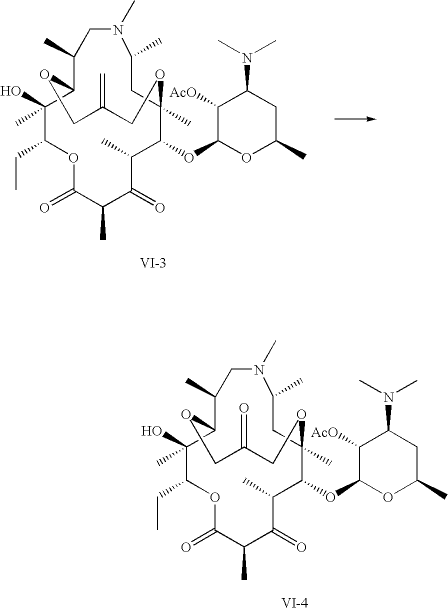

### Step 6c

A solution of VI-3 (24 mg, 0.035 mmol) and camphorsulfonic acid (16.3 mg, 0.07 mmol) in methanol (2.0 mL) was cooled in a dry ice/acetone bath. Ozone was bubbled into the solution for 10 minutes after a blue color was first observed. Oxygen was bubbled through the solution until the blue color dissipated. Dimethylsulfide (0.026 mL, 0.35 mmol) was added and the reaction was brought to room temperature. After 18 hours, the solvent was evaporated and the residue was loaded onto a 1 mL silica colun which was eluted with a mixture of 60:10:1 methylene chloride/methanol/concentrated ammonium hydroxide. The product fractions were combined and evaporated to give VI-4 as a solid (32 mg).

MS m/e (M+H)=717.75 (MeOH ketal).

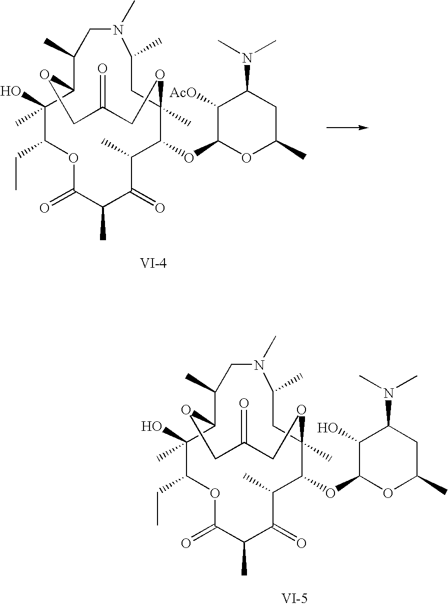

### Step 6d

A solution of VI-4 (32 mg) in methanol (1.5 mL) was placed in a 40° C. oil bath for 6 hours. The solvent was evaporated to give VI-5 as an oil (29 mg).

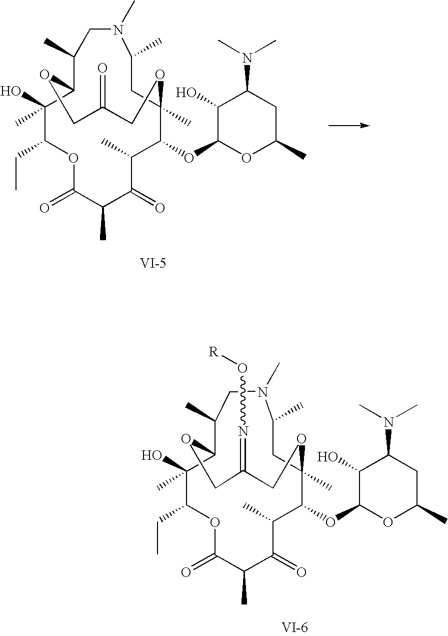

### Step 6e: R=benzyl

A solution of VI-5 (9 mg, 0.014 mmol), O-benzylhydroxylamine hydrochloride (4.5 mg, 0.028 mmol) and pyridine (0.0045 mL, 0.056 mmol) in ethanol (0.5 mL) was stirred at room temperature for 2 hours. The solvent was evaporated and the residue was placed on a 1 mL silica column which was eluted with a mixture of 60:10:1 methylene chloride/methanol/concentrated ammonium hydroxide to give VI-6 (7 mg) as a mixture of a major and minor oxime isomers. The oxime isomers could be separated by preparative silica plate chromatography (1:1 acetone/hexanes).

MS m/e (M+H)=748.77.

### Step 6f: R=[2-(pyrazol-1-yl)pyrid-5-yl]methyl

Hydrazine hydrate (1.2 uL, 0.025 mmol) was added to a solution of N-phthaloyl-O-2-pyrazolopyridyl-5-methyl-hydroxylamine (10 mg, 0.031 mmol) in ethanol (0.3 mL) and the mixture was heated in a 40° C. oil bath 3 hours. After cooling to room temperature, acetic acid (4.3 uL, 0.075 mmol) and VI-5 (16.9 mg, 0.025 mmol) were added and the mixture was stirred in a 40° oil bath for 18 hours. The solvent was evaporated and the residue was placed on a 1 mL silica column which was eluted with a mixture of 60:10:1 methylene chloride/methanol/concentrated ammonium hydroxide to give VI-6 (16 mg) as a mixture of a major and minor oxime isomers. The oxime isomers could be separated by preparative silica plate chromatography (1:1 acetone/hexanes).

MS m/e (M+H)=815.82.

### Example 7

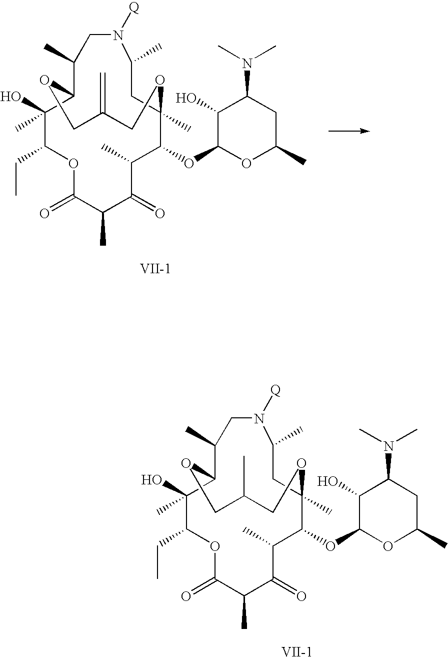

To a solution of compound IV-1 (Q=H) in acetic acid is added 5 wt. % PtO₂ and the resulting heterogenous mixture is placed under a hydrogen atmosphere and is stirred for 1-5 days. The product V-1 (Q=H) is isolated by filtration and concentration and is then purified by flash chromatography. In analogous fashion compounds V-1 (Q=CH₃) and V-1 (Q=C₃H₇) can be prepared from the appropriate IV-1.

### Example 8

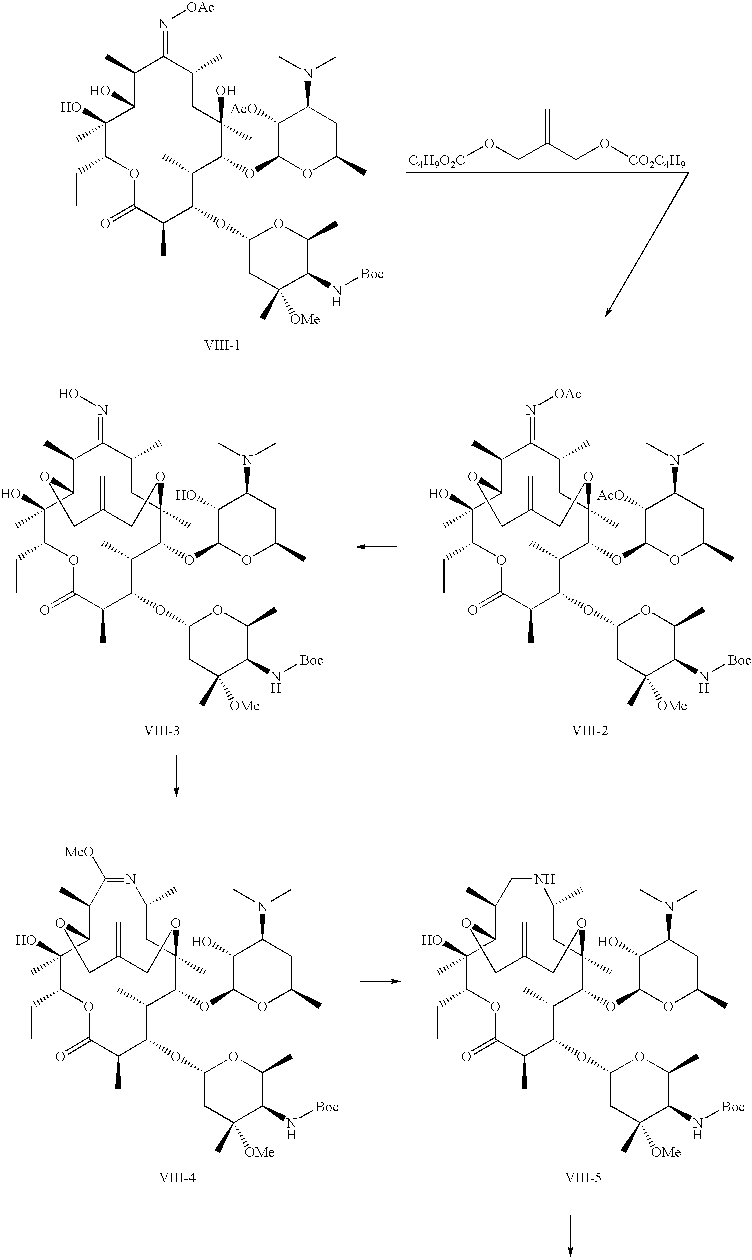

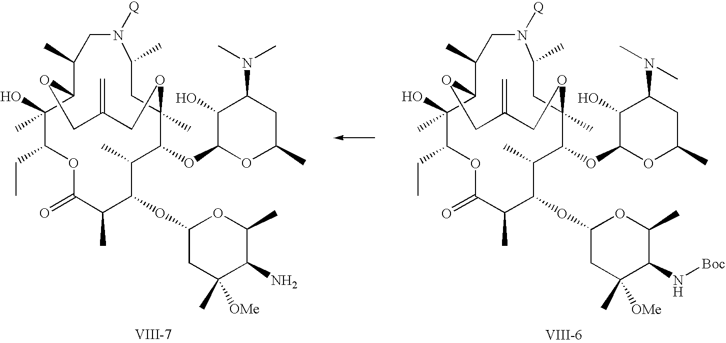

A solution of Compound VIII-1 (1 eq) in THF under nitrogen is treated sequentially with bis-(2-t-butoxycarbonyl)-2-methylene-1,3-propanediol (1.2 eq) and 1,4-bis(diphenylphosphino)butane (0.1 eq) and Pd2(dba)₃ (0.05 eq) and the mixture is refluxed until the reaction is complete. The solvent is then removed and the residue is purified by chromatography to afford compound VIII-2. Compound VIII-2 is then treated in a manner analogous to that described above in Example 1 to afford sequentially compounds VIII-3, VIII-4, and VIII-5. Compound VIII-5 can be alkylated if desired to compound VIII-6 by the procedures described in Examples 2 and 3. A solution of compound VIII-6 in CH₂Cl₂ can be treated with 1-10% by volume trifluoroacetic acid and the mixture allowed to stir until the reaction is complete. The reaction mixture can be neutralized with aqueous NaHCO₃ and the product isolated by extraction with CH₂Cl₂ and then purified chromatographically to give compound VIII-7.

Although the invention has been described with respect to various preferred embodiments, it is not intended to be limited thereto, but rather those skilled in the art will recognize that variations and modifications may be made therein which are within the spirit of the invention and the scope of the appended claims.

What is claimed is:

1. A compound of Formula I, or a pharmaceutically acceptable salt or ester or prodrug thereof: 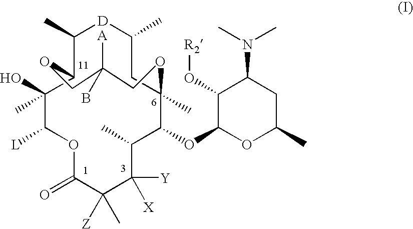
wherein:
A is
i) —OH;
ii) —ORₚ, where Rₚ is a hydroxy protecting group;
iii) —R₁, where R₁ is aryl, substituted aryl, heteroaryl, or substituted heteroaryl;
iv) —OR₁, where R₁ is as previously defined;
v) —R₂, where R₂ is
(a) hydrogen;
(b) halogen;
(c) —C₁-C₆ alkyl containing 0, 1, 2, or 3 heteroatoms selected from O, S or N, optionally substituted with one or more substituents selected from halogen, aryl, substituted aryl, heteroaryl, or substituted heteroaryl;
(d) —C₂-C₆ alkenyl containing 0, 1, 2, or 3 heteroatoms selected from O, S, or N, optionally substituted with one or more substituents selected from halogen, aryl, substituted aryl, heteroaryl, or substituted heteroaryl; or
(e) —C₂-C₆ alkynyl containing 0, 1, 2, or 3 heteroatoms selected from O, S or N, optionally substituted with one or more substituents selected from halogen, aryl, substituted aryl, heteroaryl, or substituted heteroaryl;
vi) —OR₂, where R₂ is previously defined;
vii) —S(O)ₙR₁₁, where n=0, 1 or 2, and R₁₁ is R₁ or R₂, where R₁ and R₂ are as previously defined;
viii) —CNHC(O)R₁₁, where R₁₁ is as previously defined;
ix) —NHC(O)NHR₁₁, where R₁₁ is as previously defined;
x) —NHS(O)₂R₁₁, where R₁₁ is as previously defined;
xi) —NR₁₄R₁₅, where R₁₄ and R₁₅ are each independently R₁₁, where R₁₁ is as previously defined; or
xii) —NHR₃, where R₃ is an amino protecting group;
B is
i) hydrogen;
ii) deuteriurn;
iii) halogen;
iv) —OH;
v) —R₁, where R₁ is as previously defined;
vi) —R₂, where R₂ is as previously defined; or
vii) —ORₚ, where Rₚ is as previously defined, provided that when B is halogen, —OH or ORₚ, A is R₁ or R₂, where R₁ and R₂ are previously defined;
or, alternatively, A and B taken together with the carbon atom to which they are attached are
i) C═O;
ii) C(OR₂)₂, where R₂ is as previously defined;
iii) C(SR₂)₂, where R₂ is as previously defined;
iv) C[—O(CH₂)ₘ]₂, where m=2 or 3;
v) C[—S(CH₂)ₘ]₂, where m is as previously defined;
vi) C═CHR₁₁, where R₁₁ is as previously defined;
vii) C═N—O—R₁₁, where R₁₁ is as previously defined;
viii) C═NNHR₁₁, where R₁₁ is as previously defined;
ix) C═NNHC(O)R₁₁, where R₁₁ is as previously defined;
x) C═NNHC(O)NHR₁₁, where R₁₁ is as previously defined;
xi) O═NNHS(O)₂R₁₁, where R₁₁ is as previously defined;
xii) C═NNHR₃, where R₃ is as previously defined; or
xiii) C═NR₁₁, where R₁₁ is as previously defined;
L is
i) —CH₃;
ii) —CH₂CH₃;
iii) —CH(OH)CH₃;
iv) —C₁-C₆ alkyl, optionally substituted with one or more substituents selected from aryl, substituted aryl, heteroaryl, or substituted heteroaryl;
v) —C₂-C₆ alkenyl, optionally substituted with one or more substituents selected from aryl, substituted aryl, heteroaryl, or substituted heteroaryl; or
vi) —C₂-C₆ allyl, optionally substituted with one or more substituents selected from aryl, substituted aryl, heteroaryl, or substituted heteroaryl;
D is —CH₂N(O)—, —C(O)N(R′)—, or —C(OR′)═N—, wherein R′ is R₁₁ as previously defined;
Q is
i) hydrogen;
ii) —C₁-C₁₂-alkyl, C₃-C₁₂-alkenyl, or C₃-C₁₂-alkynyl, all optionally substituted with one, two or three substituents independently selected from:
(a) halogen;
(b) —OR₆, wherein R₆ is selected from:
1. hydrogen;
2. —C₁-C₁₂-alkyl containing 0, 1, 2, or 3 heteroatoms selected from O, S or N, optionally substituted with one, two, or three substituents independently selected from aryl, substituted aryl, heteroaryl, or substituted heteroaryl;
3. aryl;
4. substituted aryl;
5. heteroaryl; and
6. substituted heteroaryl;
(c) —NR₄R₅, where R₄ and R₅ are each independently R₆, where R₆ is as previously defined, or in the alternative R₄ and R₅, together with the atom to which they are attached, form a heterocycloalkyl or substituted heterocycloalkyl moiety;
(d) —N—O—R₆, where R₆ is as previously defined;
(e) —R₁, where R₁ is as previously defined;
(f) C₃-C₈-cycloalkyl;
(g) substituted —C₃-C₈-cycloalkyl;
(h) heterocycloalkyl;
(i) substituted heterocycloalkyl;
(j) —NHC(O)R₆, where R₆ is as previously defined;
(k) —NHC(O)OR₇, where R₇ is selected from:
1. —C₁-C₁₂-alkyl containing 0, 1, 2, or 3 heteroatoms selected from O, S or N, optionally substituted with one, two, or three substituents independently selected from aryl, substituted aryl, heteroaryl, or substituted heteroaryl;
2. aryl;
3. substituted aryl;
4. heteroaryl; or
5. substituted heteroaryl;
(l) —NHC(O)NR₄R₅, where R₄ and R₅ are as previously defined;
(m) —OC(O)NR₄R₅, where R₄ and R₅ are as previously defined;
(n) —OC(O)R₇, where R₇ is as previously defined;
(o) —OC(O)OR₇, where R₇ is as previously defined;
(p) —OC(O)NR₄R₅, where R₄ and R₅ are as previously defined,
(q) —C(O)R₆, where R₆ is as previously defined,
(r) —CO₂R₆, where R₆ is as previously defined, or
(s) —C(O)NR₄R₅, where R₄ and R₅ are as previously defined;
X is hydrogen;
Y is
i) hydrogen;
ii) —OH;
iii) —ORₚ, where Rₚ is as previously defined;
iv) —OR₁₁, where R₁₁ is as previously defined;
v) —OC(O)R₁₁, where R₁₁ is as previously defined;
vi) —OC(O)NHR₁₁, where R II is as previously defined;
vii) —S(O)ₙR₁₁, where n and R₁₁ are as previously defined;
viii) 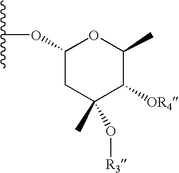
(1) where R₃″ is hydrogen or methyl; R₄″ is hydrogen or Rₚ, where Rₚ is as previously defined;
ix) 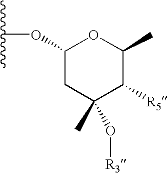
(1) where R₃″ is as previously defined; R₅″ is NH₂ or Rₐₘ, where Rₐₘ is protected amino;
or, in the alternative, X and Y are combined together to form oxo;
Z is
i) hydrogen;
ii) methyl; or
iii) halogen; and
R₂′ is hydrogen or Rₚ, where Rₚ, is as previously defined.

2. A compound according to claim 1, or a pharmaceutically acceptable salt or ester or prodrug thereof, wherein D is —CH₂N(Q)-.

3. A compound according to claim 1, or a pharmaceutically acceptable salt or ester or prodrug thereof, wherein D is CH₂N(Q)-; X is hydrogen; and Y is 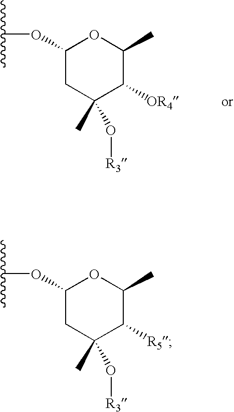
wherein R₃″, R₄″ and R₅″ are each as defined in claim 1.

4. A compound according to claim 3, or a pharmaceutically acceptable salt or ester or prodrug thereof, wherein Y is

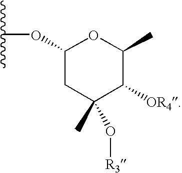

5. A compound according to claim 1, or a pharmaceutically acceptable salt or ester or prodrug thereof, wherein D is —N(Q)CH₂— and X and Y taken together are oxo.

6. A compound according to claim 1, or a pharmaceutically acceptable salt or ester or prodrug thereof, wherein D is —N═CH(OR′)—, wherein R′ is as defined in claim 1.

7. A compound according to claim 1, or a pharmaceutically acceptable salt or ester or prodrug thereof, wherein D is —C(O)N(R′)′, wherein R′ is as defined in claim 1.

8. A compound according to claim 1, or a pharmaceutically acceptable salt or ester or prodrug thereof, selected from the group consisting of:
(i) a compound of Formula I, wherein A and B taken together with the carbon atom to which they are attached is C═CH₂, D is —CH₂N(Q)-, Q=X=Z=H, Y=OH, L=CH₂CH₃, R₂′=Ac;
(ii) a compound of Formula I, wherein A and B taken together with the carbon atom to which they are attached is C═CH₂, D=—CHN(Q)-, Q=Z=H, X and Y taken together are oxo, L=CH₂CH₃, R₂′=H;
(iii) a compound of Formula I, wherein A and B taken together with the carbon atom to which they are attached are C═CH₂, D=—CH₂N(Q)-, Q=CH₃, X=Z=H, Y=OH, L=CH₂CH₃, R₂′=H;
(iv) a compound of Formula I, wherein A and B taken together with the carbon atom to which they are attached are C═CH₂, D=—CH₂N(Q)-, Q=CH₃, Z=H, X and Y taken together are oxo, L=CH₂CH₃, R₂′=H;
(v) a compound of Formula I, wherein A=H, B=CH₃, D=—CH₂N(Q)-, Q=X=Z=H, Y=OH, L=CH₂CH₃, R₂′=Ac;
(vi) a compound of Formula I, wherein A=H, B=CH₃, D=—CH₂N(Q)-, Q=X=Z=H, Y=OH, L=CH₂CH₃, R₂′=H;
(vii) a compound of Formula I, wherein A=H, B=CH₃, D=—CHN(Q)-, Q=Z=H, X and Y taken together are oxo, L=CH₂CH₃, R₂′=H;
(viii) a compound of Formula I, wherein A→H, B=CH₃, D=—CH₂N(Q)-, Q=CH₃, X=Z=H, Y=OH, L=CH₂CH₃, R₂′=H;
(ix) a compound of Formula I, wherein A=H, B=CH₃, D=—CHN(Q)-, Q=CH₃, Z H, X and Y taken together are oxo, L=CH₂CH₃, R₂′=H;
(x) a compound of Formula I, wherein A=H, B=CH₃, D=—(C═NOH)—, X=Z=H, Y= 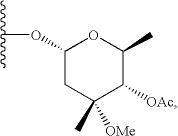
L=CH₂CH₃, R₂′=Ac;
(xi) a compound of Formula I, wherein A=H, B=CH₃, D=—C(═O)NH—, X=
Z=H, Y= 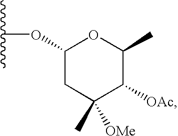
L=CH₂CH₃, R₂′=Ac;
(xii) a compound of Formula I, wherein A=H, B=CH₃, D=—C(═O)NH—, X=Z=H, Y= 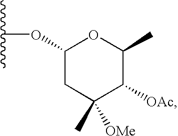
L=CH₂CH₃, R₂′=H;
(xiii) a compound of Formula I, wherein A and B taken together with the carbon atom to which they are attached ═C═CH₂, D=—CHN(Q)-, Q=CH₂-Ph, Z=X═H, Y=OH, L=CH₂CH₃, R₂′=H;
(xiv) a compound of Formula I, wherein A and B taken together with the carbon atom to which they are attached ═C═CH₂, D=—CH₂N(Q)-, Q=CH₂-Ph, Z=H, X and Y are taken together are oxo, L=CH₂CH₃, R₂′=H;
(xv) a compound of Formula I, wherein A and B taken together with the carbon atom to which they are attached ═C═CH₂, D=—CH₂N(Q)-, Q=CH₂-(2-pyridyl), Z=X═H, Y=OH, L=CH₂CH₃, R₂′=H;
(xvi) a compound of Formula I, wherein A and B taken together with the carbon atom to which they are attached ═C═CH₂, D=—CH₂N(Q)-, Q=CH₂-(2-pyridyl), Z=H, X and Y taken together are oxo, L=CH₂CH₃, R₂′=H;
(xvii) a compound of Formula I, wherein A and B taken together with the carbon atom to which they are attached ═C═CH₂, D=—CH₂N(Q)-, Q=CH₂-(3-quinolyl), Z=H, X and Y taken together are oxo, L CH₂CH₃, R₂′=H;
(xviii) a compound of Formula I, wherein A and B taken together with the carbon atom to which they are attached ═C═CH₂, D-CH₂N(Q)-, Q=CH₂-(3-quinolyl), Z=H, X and Y taken together are oxo, L=CH₂CH₃, R₂′=H;
(xix) a compound of Formula I, wherein A and B taken together with the carbon atom to which they are attached ═C═CH₂, D=—CH₂N(Q)-, Q=CH₂(CH═CH)-Ph, Z=X═H, Y=OH, L=CH₂CH₃, R₂′=H;
(xx) a compound of Formula I, wherein A and B taken together with the carbon atom to which they are attached ═C═CH₂, D=—CHN(Q)-, Q=CH₂(CH═CH)-Ph, Z=H, X and Y taken together are oxo, L=CH₂CH₃, R₂′=H;
(xxi) a compound of Formula I, wherein A and B taken together with the carbon atom to which they are attached ═C═CH₂, D=—CH₂N(Q)-, Q=CH₂CH—CH-(2-pyridyl), Z=X═H, Y=OH, L=CH₂CH₃, R₂′=H;
(xxii) a compound of Formula I, wherein A and B taken together with the carbon atom to which they are attached ═C═CH₂, D=—CHN(Q)-, Q=CH₂CH═CH-(2-pyridyl), Z=H, X and Y taken together are oxo, L=CH₂CH₃, R₂′=H;
(xxiii) a compound of Formula I, wherein A and B taken together with the carbon atom to which they are attached ═C═CH₂, D=—CH₂N(Q)-, Q=CH₂C═C-(3-quinolyl), Z=H, X and Y taken together are oxo, L=CH₂CH₃, R₂′=H;
(xxiv) a compound of Formula I, wherein A and B taken together with the carbon atom to which they are attached ═C═CH₂, D=—CH₂N(Q)-, Q=CH₂C═C-(3-quinolyl), Z=H, X and Y taken together are oxo, L=CH₂CH₃, R₂′=H;
(xxv) a compound of Formula I, wherein A and B taken together with the carbon atom to which they are attached ═C═CH—CH═CH-Ph, D=—CH₂N(Q)-, Q=CH₃, Z=H, X and Y taken together are oxo, L=CH₂CH₃, R₂′=H;
(xxvi) a compound of Formula I, wherein A and B taken together with the carbon atom to which they are attached ═C═CH—CH═CH-(3-pyridyl), D=—CH₂N(Q)-, Q=CH₃, Z=H, X and Y taken together are oxo, L=CH₂CH₃, R₂′=H;
(xxvii) a compound of Formula I, wherein A and B taken together with the carbon atom to which they are attached ═C═CH—CH═CH-(3-quinolyl), D=—CH₂N(Q)-, Q=CH₃, Z=H, X and Y taken together are oxo, L=CH₂CH₃, R₂′=H;
(xxviiii) a compound of Formula I, wherein A and B taken together with the carbon atom to which they are attached ═C═CH-(3-quinolyl), D=—CH₂N(Q)-, Q=CH₃, Z=H, X and Y taken together are oxo, L=CH₂CH₃, R₂′=H; and
(xxix) a compound of Formula I, wherein A and B taken together with the carbon atom to which they are attached ═C═CH-Ph, D=—CH(O)—, Q=CH₃, Z=H, X and Y taken together are oxo, L=CH₂CH₃, R₂′=H;
(xxx) a compound of Formula I, wherein A and B taken together with the carbon atom to which they are attached is C═CH₂, D is —CH₂N(Q)-, Q=X=Z=H, Y=OH, L=CH₂CH₂CH₃, R₂′=H;
(xxxi) a compound of Formula I, wherein A and B taken together with the carbon atom to which they are attached is C═CH₂, D is —CH₂N(Q)-, Q=CH₂CH₂CH₃, X=Z=H, Y=OH, L=CH₂CH₃, R₂′=H;
(xxxii) a compound of Formula I, wherein A and B taken together with the carbon atom to which they are attached is C=CH₂, D is —CH₂N(Q)-, Q=CH₂CH₂CH₃, Z=H, X and Y taken together are oxo, L=CH₂CH₃, R₂′=H;
(xxxiii) a compound of Formula I, wherein A=H, B=CH₃, D=—CH₂N(Q)-, Q=CH₂CH₂CH₃, Z=H, X and Y taken together are oxo, L=CH₂CH₂CH₃, R₂′=H;
(xxxiv) a compound of Formula I, wherein A and B taken together with the carbon atom to which they are attached is C═O, D is —CH₂N(Q)-, Q=Z=H, X and Y taken together are oxo, L=CH₂CH₃, R₂′=H;
(xxxv) a compound of Formula I, wherein A and B taken together with the carbon atom to which they are attached is C═O, D is —CH₂N(Q)-, Q=CH₃, Z=H, X and Y taken together are oxo, L=CH₂CH₃, R₂′=H;
(xxxvi) a compound of Formula I, wherein A and B taken together with the carbon atom to which they are attached is C═O, D is CH₂N(Q)-, Q=CH₂CH₂CH₃, Z=H, X and Y taken together are oxo, L=CH₂CH₃, R₂′=H;
(xxxvii) a compound of Formula I, wherein A and B taken together with the carbon atom to which they are attached is C═N—OH, D is —CH₂N(Q)-, Q=Z=H, X and Y taken together are oxo, L=CH₂CH₃, R₂′=H;
(xxxviii) a compound of Formula I, wherein A and B taken together with the carbon atom to which they are attached is C═N—OH, D is —CH₂N(Q)-, Q=CH₃, Z=H, X and Y taken together are oxo, L=CH₂CH₃, R₂′=H;
(xxxix) a compound of Formula I, wherein A and B taken together with the carbon atom to which they are attached is C═N—OH, D is —CH₂N(Q)-, Q=CH₂CH₂CH₃, Z=H, X and Y taken together are oxo, L=CH₂CH₃, R₂′=H;
(xl) a compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=Z=H, X and Y taken together are oXo, L=CH₂CH₃, R₂′=H;
(xli) a compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=CH₃, Z=H, X and Y taken together are oxo, L=CH₂CH₃, R₂′=H;
(xlii) a compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=CH₂CH₂CH₃, Z=H, X and Y taken together are oxo, L=CH₂CH₃, R₂′=H;
(xliii) a compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=Z=H, X and Y taken together are oxo, L=CH₂CH₃, R₂′=H;
(xliv) a compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=CH₃, Z=H, X and Y taken together are oxo, L=CH₂CH₃, R₂′=H;
(xlv) a compound of Formula I, wherein A and B taken together with the carbon atom to which they are attached is C═N—O—R₁₁, R₁₁₌₅-[2-(pyrazol-1-yl)pyrid-5-yl]methyl, D is —CH₂N(O)—, Q=CH₂CH₂CH₃, Z=H, X and Y taken together are oxo, L=CH₂CH₃, R₂′=H;
(xlvi) a compound of Formula I, wherein A and B taken together with the carbon atom to which they are attached is C═CH₂, D is —CH₂N(Q)-, Q=X=Z=H, Y= 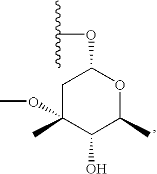
L CH₂CH₃, R₂′=H;
(xlvii) a compound of Formula I, wherein A and B taken together with the carbon atom to which they are attached is C═CH₂, D is —CH₂N(O), Q CH₃, X=Z=H, Y= 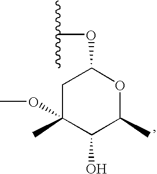
L=CH₂CH₃, R₂′=H;
(xlviii) a compound of Formula I, wherein A and B taken together with the carbon atom to which they are attached is C═CH₂, D is —CH₂N(Q)-, Q=CH₂CH₂CH₃, X=Z H, Y 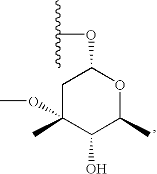
L=CH₂CH₃, R₂′=H;
(xlix) a compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=X=Z=H, Y= 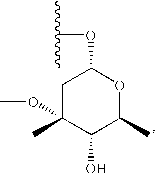
L=CH₂CH₃, R₂′=H;
(l) a compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=CH₃, X=Z=H, Y= 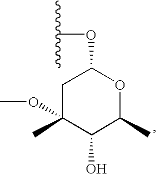
L CH₂CH₃, R₂′=H;
(li) a compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q CH₂CH₂CH₃, X Z=H, Y= 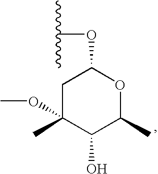
L=CH₂CH₃, R₂′=H;
(lii) a compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=X=Z=H, Y= 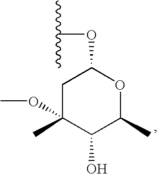
L=CH₂CH₃, R₂′=H;
(liii) a compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=CH₃, X=Z=H, Y= 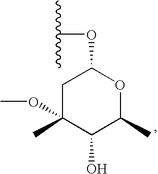
L=CH₂CH₃, R₂′=H;
(liv) a compound of Formula I, wherein A and B taken together with the carbon atom to which they are attached is C═N—OR₁₁, R₁₁=[2-(pyrazol-1-yl)pyrid-5-yl]methyl, D is —CH₂N(Q)-, Q=CH₂CH₂CH₃, X=Z=H, Y= 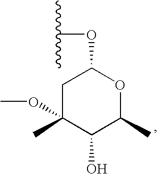
L=CH₂CH₃, R₂′ H;
(lv) a compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=X=Z=H, Y= 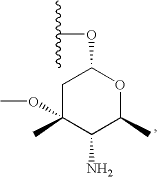
L=CH₂CH₃, R₂′=H;
(lvi) a compound of Formula I, wherein A and B taken together with the carbon atom to which they are attached is C═N—O—R₁₁, R₁₁=2-[5-(6-aminopyrid-2-yl)thien-2-yl]methyl, D is —CH₂N(Q)-, Q=CH₃, X=Z=H, Y= 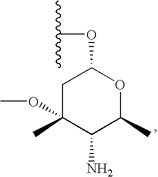
L CH₂CH₃, R₂′=H;
(lvii) a compound of Formula I, wherein A and B taken together with the carbon atom to which they are attached is C═N—O—R₁₁, R₁₁=[5-(6-aminopyrid-2-yl)thien-2-yl]methyl, D is —CH₂N(Q)-, Q=CH₂CH₂CH₃, X=Z=H, Y= 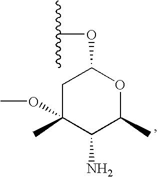
L=CH₂CH₃, R₂′=H;
(lviii) a compound of Formula I, wherein A and B taken together with the carbon atom to which they are attached is C═N—O—R₁₁, R₁₁=5-[2-(pyrazol-1-yl)pyrid-5-yl]methyl, D is —CH₂N(O)—, Q=X=Z=H, Y= 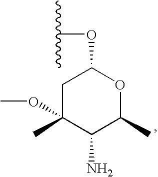
L=CH₂CH₃, R₂′=H;
(lix) a compound of Formula I, wherein A and B taken together with the carbon atom to which they are attached is C═N—O—R₁₁, R₁₁=[2-(pyrazol-1-yl)pyrid-5-yl]methyl, D is —CH₂N(Q)-, Q=C3, X=Z=H, Y= 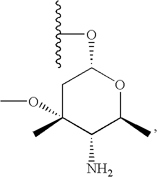
L=CH₂CH₃, R₂′=H; and
(lx) a compound of Formula I, wherein A and B taken together with the carbon atom to which they are attached is C═N—OR₁₁, R₁₁=[2-(pyrazol-1-yl)pyrid-5-yl]methyl, D is —CH₂N(Q)-, Q=CH₂CH₂CH₃, X=Z=H, Y= 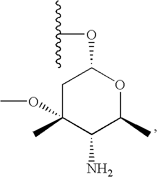
L=CH₂CH₃, R₂′=H.

9. A compound according to claim 1, or a pharmaceutically acceptable salt or ester or prodrug thereof, selected from the group consisting of:

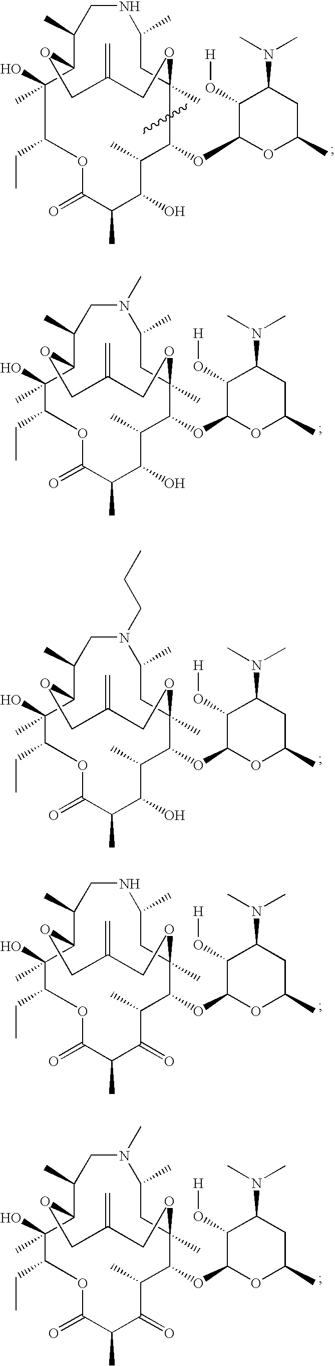

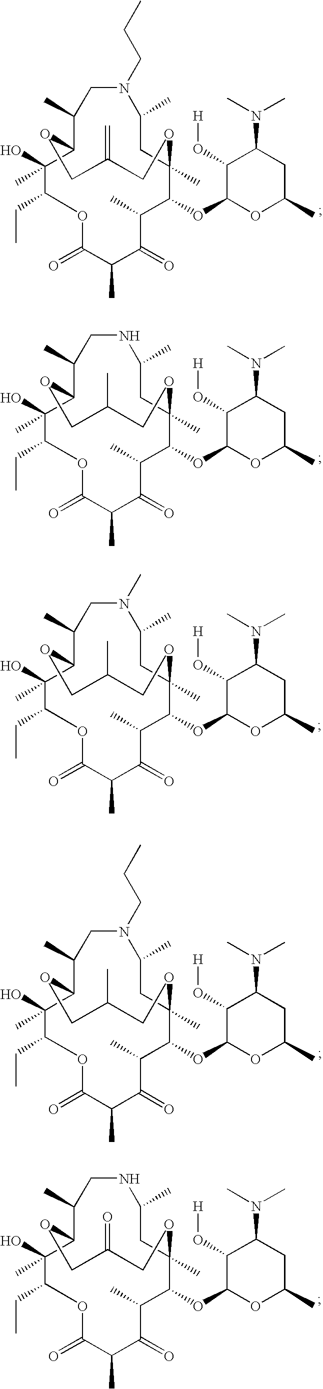

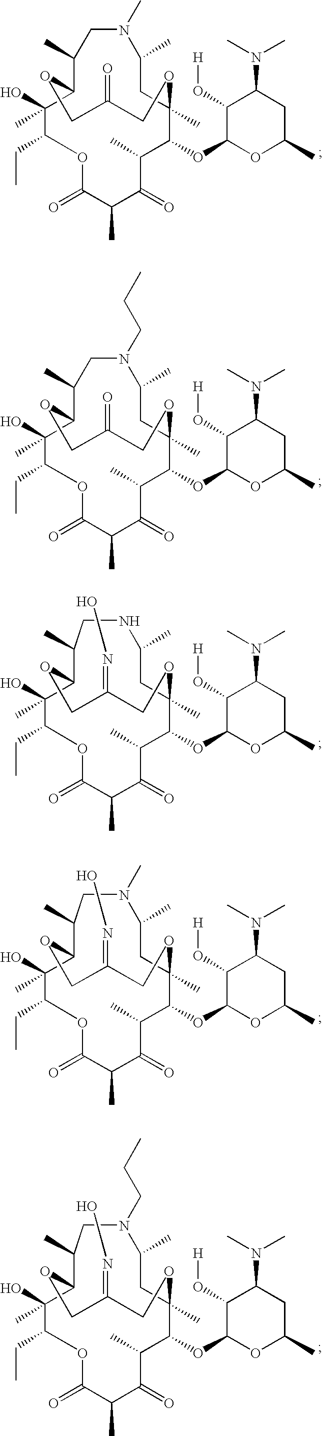

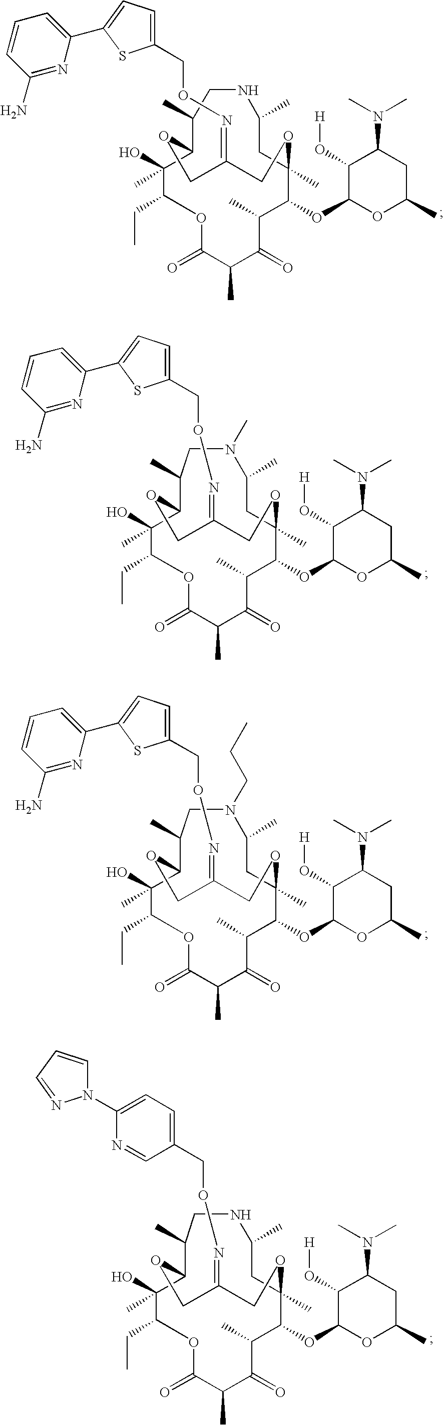

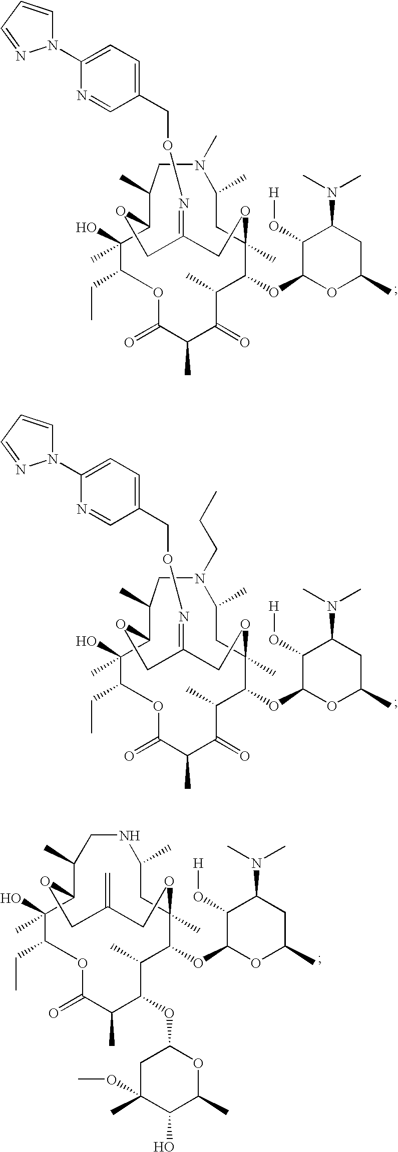

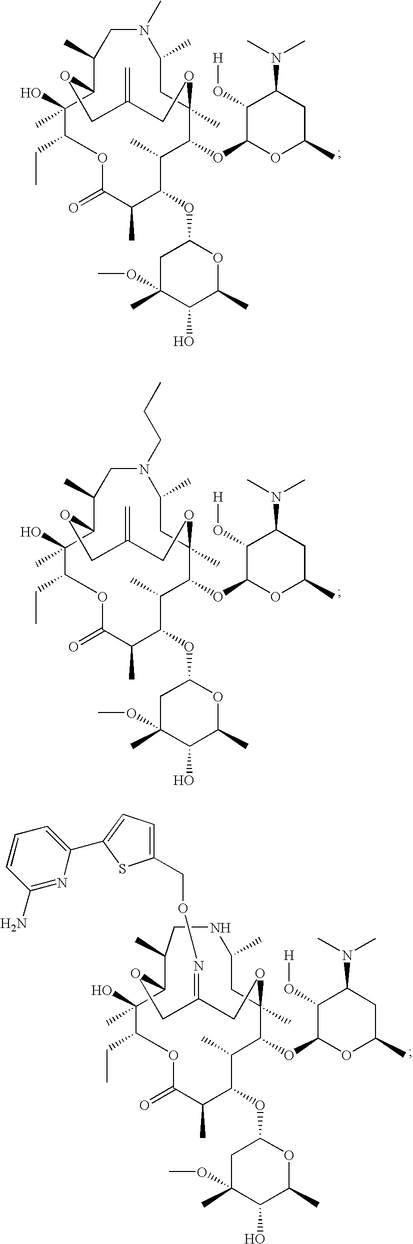

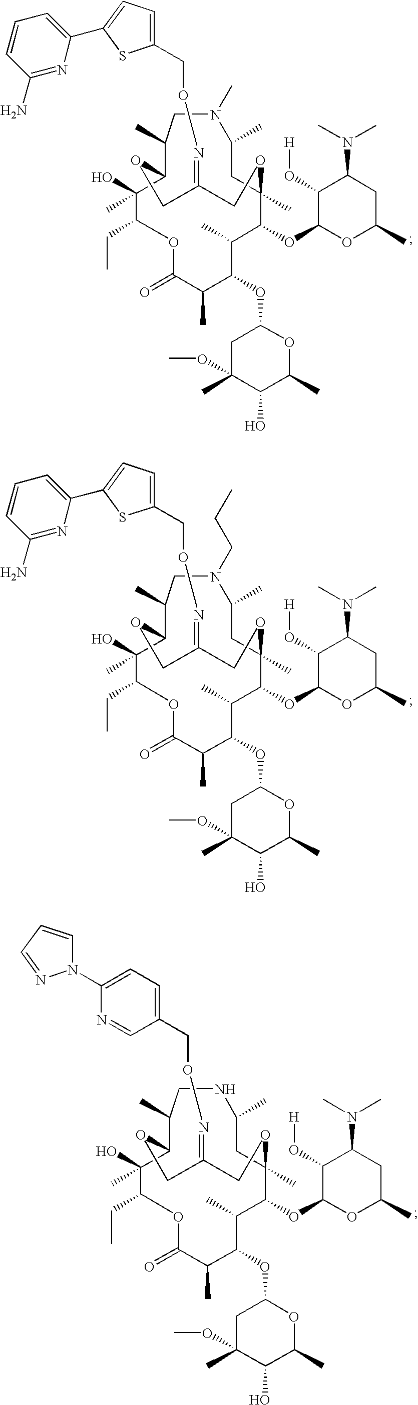

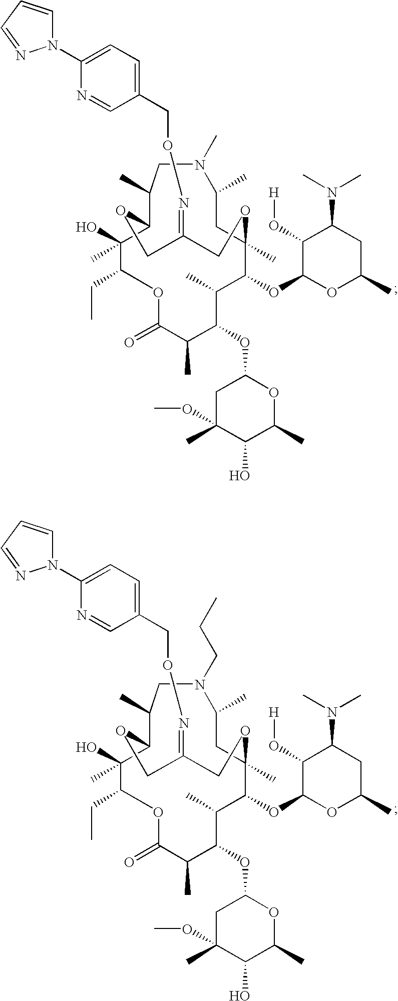

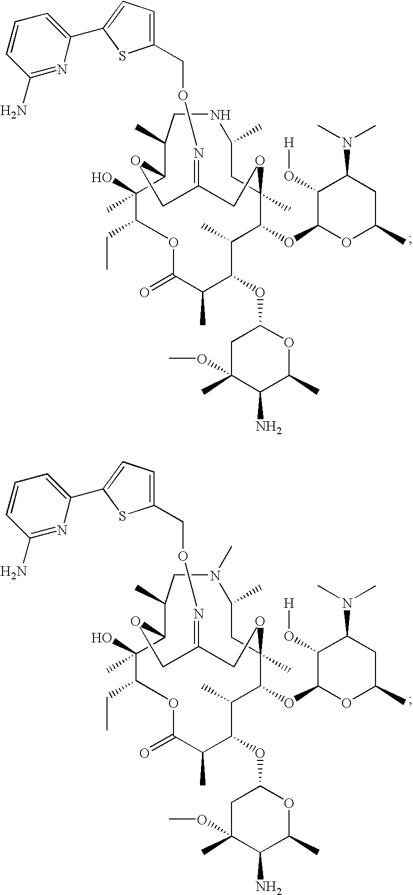

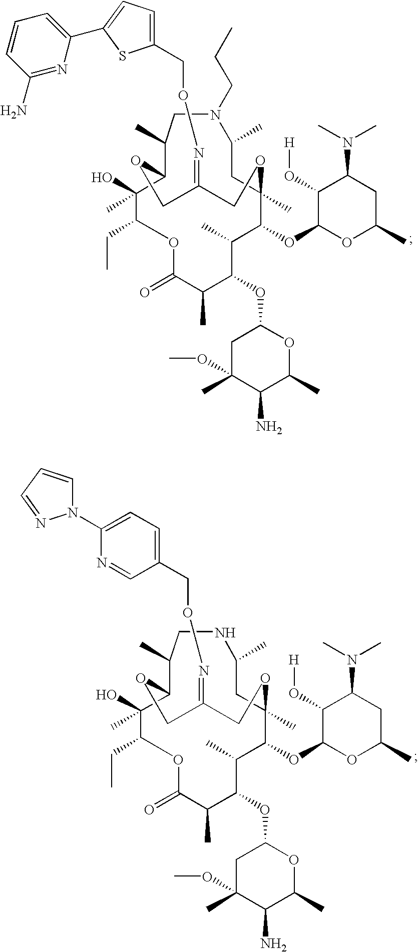

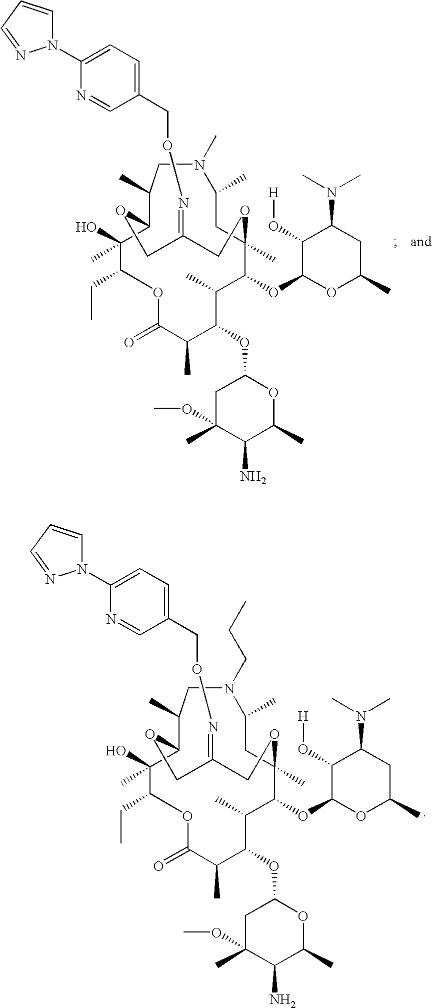

10. A pharmaceutical composition comprising:
(i) a compound of Formula I as defined in claim 1, or a pharmaceutically acceptable salt or ester or prodrug thereof, in an amount effective for treating or preventing a bacterial infection; and
(ii) a pharmaceutically acceptable carrier.

11. A pharmaceutical combination of
(i) a compound of Formula I as defined in claim 1, or a pharmaceutically acceptable salt or ester or prodrug thereof, and
(ii) an antibacterial agent other than a compound of Formula I or a salt, ester or prodrug thereof;
wherein the compound of Formula I or its pharmaceutically acceptable salt or ester or prodrug and the antibacterial agent are each employed in an amount that renders the combination effective for treating or preventing a bacterial infection.

12. A method for treating or preventing a bacterial infection in a subject in need thereof, which comprises administering to the subject a therapeutically or prophylactically effective amount of a compound according to claim 1, or a pharmaceutically acceptable salt or ester or prodrug thereof.

13. A method for treating or preventing a bacterial infection in a subject in need thereof, which comprises administering to the subject a therapeutically or prophylactically effective amount of a pharmaceutical composition according to claim 10.

14. A method for treating or preventing a bacterial infection in a subject in need thereof, which comprises administering to the subject a therapeutically or prophylactically effective amount of a pharmaceutical combination according to claim 11.

15. A process for the preparation of a compound of formula: 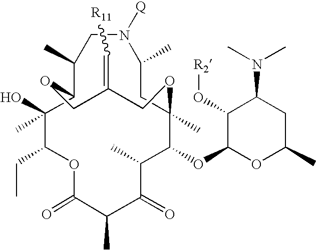
wherein Q and R₂′ are each as defined in claim 1, which comprises:
(1) reacting a compound of formula: 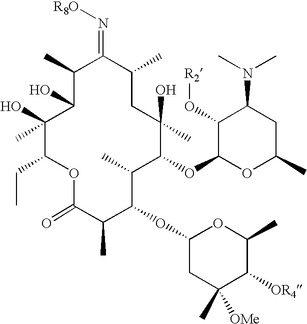
with an alkylating agent of formula: 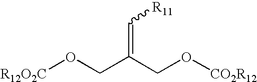
in the presence of a phosphine ligand and Pd(0) catalyst under reflux conditions to prepare a compound of the Formula: 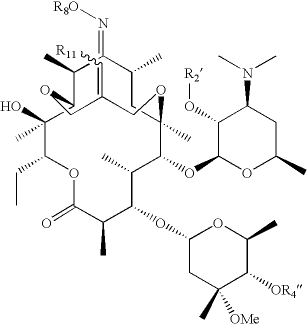
wherein:
R₈ is
a. hydrogen,
b. —CH₂O(CH₂)₂OCH₃,
c. —CH₂—O—(CH₂O)ₙCH₃ where n is zero, 1 or 2;
d. —C₁—Cl₂ alkyl, optionally substituted with one or more substituents selected from aryl, substituted aryl, heteroaryl and substituted heteroaryl;
e. —C₃—Cl₂ cycloalkyl;
f. —C(O)—C₁-C₁₂alkyl;
g. —C(O)—C₃-C₁₂ cycloalkyl;
h. —C(O)—R₁, where R₁ is as previously defined; or
i. —Si(Rₐ)(R_{b})(R_{c}), wherein Rₐ, R_{b} and R_{c} are each independently selected from C₁-C₁₂ alkyl, aryl and substituted aryl;
R₂′ and R₄″ are as previously defined in claim 1; and
R₁₁ is as defined in claim 1 and R₁₂ is C₁-C₁₂ alkyl;
(2) treating the compound obtained in step (1) with an aqueous base to obtain the Z-oxime of formula: 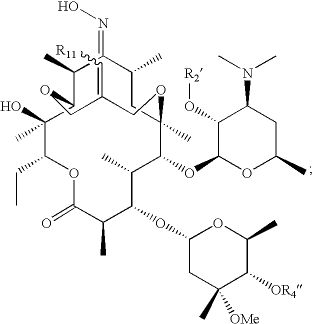
(3) reacting the compound prepared in step (2) with an oxime activating agent and quenching with methanol to prepare a compound of formula: 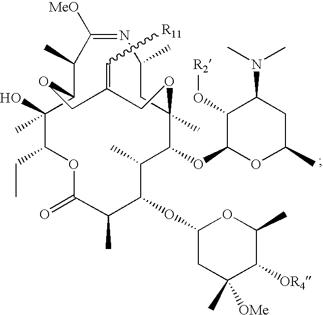
(4) reacting the compound prepared in step (3) with a reducing agent to prepare compound of formula: 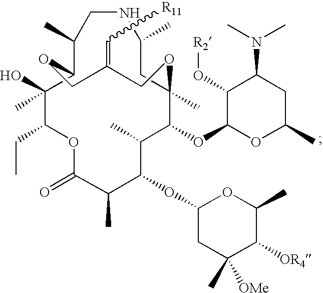
(5) reacting the compound prepared in step (4) with a mild acid to prepare a compound of formula: 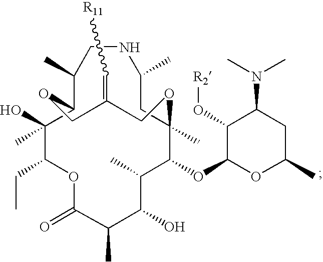
(6) reacting the compound prepared in step (5) with an agent containing the group Q selected from the group consisting of an alkylating agent, an alkyl halide in the presence of a base, and an aldehyde via reductive amination in the presence of NaCNBH₃ to prepare a compound of formula: 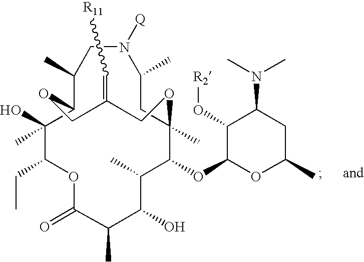
(7) oxidizing the hydroxyl in the 3 position of the compound prepared in step (6) via Dess-Martin oxidation, Corey-Kim oxidation, or a Moffat oxidation to prepare a compound of formula:

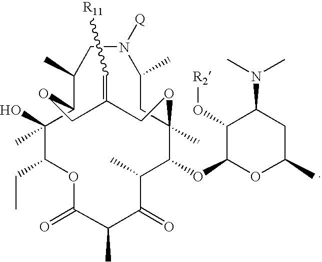

16. A process of preparing compounds of formula: 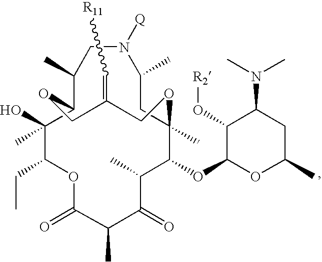
which comprises
(a) reacting a compound of formula: 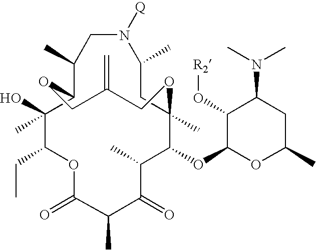
with CH₂=CH—R₁₁ in the presence of a ruthenium catalyst;
wherein Q, R₂′, and R₁₁ are each as defined in claim 1.

17. A process of preparing compounds of formula: 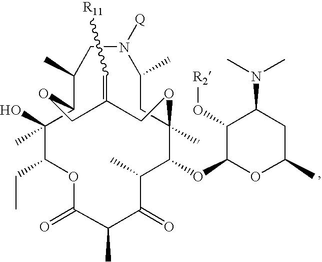
which comprises
(a) reacting a compound of formula: 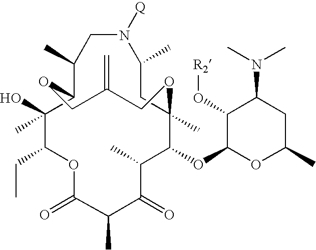
with R₁₁-halide under Heck coupling conditions using a palladium catalyst optionally with a phosphine ligand;
wherein Q and R₂′ are each as defined in claim 1; and R₁₁ is aryl, substituted aryl, or C₁-C₆ alkyl substituted with aryl or substituted aryl.

18. A process of preparing a compound of the Formula: 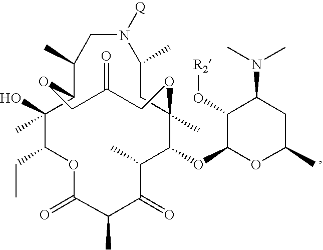
which comprises:
(a) performing ozonolysis on a compound of formula: 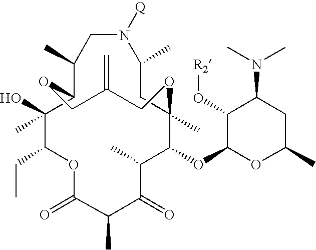
wherein Q and R₂′ are each as defined in claim 1.

19. A process of preparing a compound of formula: 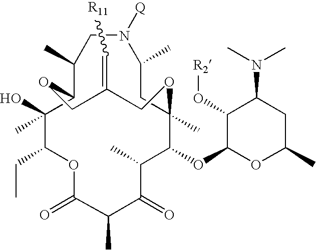
which comprises:
(a) reacting a compound of formula: 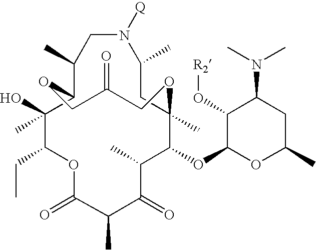
with a phosphoylid under Wittig conditions;
wherein Q, R₂′, and R₁₁ are as defined in claim 1.

20. A process of preparing a compound of formula: 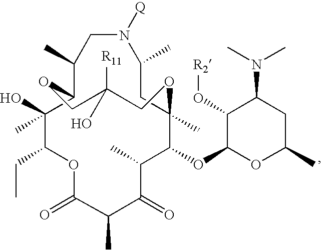
which comprises:
(a) reacting a compound of formula: 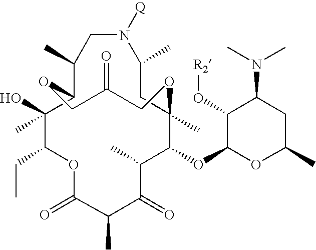
with a Grignard reagent containing the R₁₁ group;
wherein Q, R₂′, and R₁₁ are as defined in claim 1.
